(19) United States
(12) Patent Application Publication
(10) Pub. No.: US 2022/0154170 A1
(43) Pub. Date: May 19, 2022

(54) PROTEIN RESIDUE MAPPING USING A COMBINATION OF DEEP MUTATIONAL SCANNING AND PHAGE DISPLAY HIGH THROUGHPUT SEQUENCING

(71) Applicant: Fred Hutchinson Cancer Research Center, Seattle, WA (US)

(72) Inventors: Julie Overbaugh, Seattle, WA (US); Meghan Garrett, Seattle, WA (US)

(73) Assignee: Fred Hutchinson Cancer Research Center, Seattle, WA (US)

(21) Appl. No.: 17/435,610

(22) PCT Filed: Feb. 28, 2020
(86) PCT No.: PCT/US20/20429

§ 371 Date: Sep. 1, 2021

Related U.S. Application Data
(60) Provisional application No. 62/812,804, filed on Mar. 1, 2019.

(51) Int. Cl. C12N 15/10

(52) U.S. Cl. CPC C12N 15/1037

(57) ABSTRACT

The current disclosure provides protein residue mapping using a combination of deep mutational scanning and phage display high throughput sequencing. The disclosed methods allow mapping of antibody epitopes and determination of changes in residues of a protein that abolish binding of the protein to a candidate binding molecule.

### CROSS-REFERENCE TO RELATED APPLICATION

This application claims priority to U.S. Provisional Patent Application No. 62/812,804 filed Mar. 1, 2019, which is incorporated by reference in its entirety as if fully set forth herein.

### STATEMENT REGARDING FEDERALLY SPONSORED RESEARCH OR DEVELOPMENT

This invention was made with government support under grant A1038518 awarded by the National Institutes of Health. The government has certain rights in the invention.

### REFERENCE TO SEQUENCE LISTING

The Sequence Listing associated with this application is provided in text format in lieu of a paper copy and is hereby incorporated by reference into the specification. The name of the text file containing the Sequence Listing is F053-0099PCT_ST25.txt. The text file is 53 KB, was created on Feb. 27, 2020 and is being submitted electronically via EFS-Web.

### FIELD OF THE DISCLOSURE

The current disclosure provides protein residue mapping using a combination of deep mutational scanning and phage display high throughput sequencing. The disclosed methods allow mapping of antibody epitopes and determination of changes in residues of a protein that abolish binding of the protein to a candidate binding molecule.

### BACKGROUND OF THE DISCLOSURE

Proteins are made of strings of amino acids with different proteins having different numbers and orders of amino acids. Proteins are essential to the functioning of cells and organisms. A powerful way to study proteins is through mutagenesis. Mutagenesis refers to altering the amino acid that naturally occurs at a position along the string of amino acids that create a given protein. Systematically altering amino acids at different positions through mutagenesis can identify those amino acids that are essential to the function of the protein. Deep mutational scanning (DMS) refers to methods of generating and characterizing hundreds of thousands of mutants or more of a given protein. More particularly, DMS can refer to altering up to each amino acid position with all possible alternative amino acids and assessing the effects of each individual substitution.

One scenario where the study of proteins is extremely beneficial is in relation to viruses and antibody binding to proteins located on viruses. For example, to combat the spread of viruses, such as influenza, human immunodeficiency virus (HIV), Ebola virus, Zika virus, and coronavirus (CoV), to name a few, scientists and doctors need tools to know when therapeutic antibodies are working against viral proteins, or conversely, when these viral proteins have developed resistance to the antibodies and pose a greater risk. For this, they need to know how these antibodies interact with viral proteins including which amino acids of the antibody and viral protein are responsible for binding between them. DMS has been applied for this purpose using infectious viral particles, which raise numerous safety and logistical concerns.

Bacteriophage (commonly referred to as phage) are viruses that infect and replicate within bacteria. Phage are not infectious for humans or other animals. Researchers have used phage display libraries to study protein:protein interactions. Historically, phage display relied on the production of very large collections of random peptides associated with their corresponding genetic blueprints (Scott et al., Science 249:386-390 (1990); Dower, Curr Biol 2:251-253 (1992); Cortese et al., Trends Biotechnol 12:262-267 (1994); Cortese et al., Curr Opin Biotechnol 7:616-621 (1996)). Presentation of the random peptides was often accomplished by constructing chimeric or fusion proteins expressed on the outer surface of the phage. This presentation made the libraries amenable to the study of binding assays in the form of biopanning (Parmley et al., Gene 73:305-318 (1988)) leading to the affinity isolation and identification of peptides with desired binding properties.

Currently, phage display enables the expression of designed proteins and peptides on the surface of phage particles, with a direct link between the genotype and the phenotype of the peptide or protein of interest. This method enables vast libraries of peptides or proteins to be screened simultaneously for their ability to interact with other molecules, such as ligands, enzyme substrates and the like.

Despite advancements in the use of DMS and phage display libraries over the last decades, there is still significant room for improvement in the ability to precisely identify amino acid residues within a protein that are essential for the protein's binding to other molecules.

### SUMMARY OF THE DISCLOSURE

The current disclosure provides protein residue mapping using a combination of deep mutational scanning (DMS) of a protein of interest expressed by a phage library; exposure of the phage library to a potential binding molecule; an efficient immunoprecipitation step to isolate bound complexes of proteins of interest and the binding molecule; and identifying the sequences of bound peptides to perform protein residue mapping. The combined process is referred to herein as Phage-DMS. Phage-DMS vastly simplifies previously used approaches to protein residue mapping and overcomes numerous bottlenecks and safety considerations associated with currently available viral protein residue mapping processes. For example, the currently disclosed methods can include an entire DMS library for a protein of interest in one experimental tube and the use of molecular barcodes is not needed. Further, the methods do not rely on the use of functional assays to determine presence or absence of protein interaction. The methods described herein can also provide more detailed information regarding linear protein residue mapping and can identify mutations that result in loss of interaction between proteins of interest and candidate binding molecules.

### BRIEF DESCRIPTION OF THE DRAWINGS

Many of the drawings submitted herein are better understood in color. Applicant considers the color versions of the drawings as part of the original submission and reserves the right to present color images of the drawings in later proceedings.

FIG. 1. The HIV virion has an envelope protein including glycoprotein 41 (gp41) and glycoprotein 120 (gp120).

FIGS. 2A-2C. Results of Binding Antibody Multiplex Assay (BAMA) for HIV-specific QA255 antibodies. (FIG. 2A) The binding of the QA255 monoclonal antibodies (mAbs) indicated at the top of each column are shown in relation to the antigen tested in the BAMA assay, which is indicated in the first two columns. The first two mAbs bind to different epitopes on HIV surface protein gp120 (Williams et al. EBioMedicine. 2015; 2:1464-1477) and served as controls. Binding to each antigen is defined by the fold increase over background (results with human immunodeficiency virus (HIV) negative plasma) and the binding is color coded as indicated to the right, with darker shades of gray indicating more binding. Light gray indicates binding was not detected above background (<2-fold). (FIGS. 2B, 2C). Binding defined by enzyme-linked immunosorbent assay (ELISA) to MN gp41 and ZA1197 gp41 ectodomain proteins. Absorbance is shown in relation to antibody concentration; the dotted line indicates the limit of detection. The key for the mAbs tested is shown to the right.

FIGS. 3A-3C. gp41-specific QA255 mAbs mediate antibody dependent cellular toxicity (ADCC) activity. Percent ADCC activity for target cells coated with either (FIG. 3A) clade B MN gp41 protein, (FIG. 3B) clade C ZA1197 gp41 ectodomain, or (FIG. 3C) clade A Q461.e2 TAIV gp140 protein is shown on the y-axis. The mAbs tested are shown on the x-axis. Results are the average of two replicates. The results are representative of studies with PBMCs from two different donors (see FIGS. 4A-4C).

FIGS. 4A-4C. gp41-specific QA255 mAbs mediate ADCC activity with PBMCs from second donor.

FIGS. 5A-5E. Results of competition ELISAs with mAbs that target known epitopes in gp41. (FIG. 5A) The mAbs used for competition experiments and their epitope targets are shown with a schematic of gp41 below. (FIGS. 5B-5E) Results of competition experiments reported as biotinylated (Bt) mAb QA255.006 (FIG. 5B), QA255.016 (FIG. 5C), QA255.067 (FIG. 5D), QA255.072 and (FIG. 5E) binding in the presence versus absence of the competitor mAbs. The tested Bt-mAb is indicated at the top of each panel, and the competitor mAbs are indicated on the x-axis. The results shown are from technical duplicates in the same experiment and are representative of at least two biological replicates.

FIG. 6. Competitor mAbs of known epitope specificity bind MN gp41 protein.

FIGS. 7A, 7B. Competition binding assay with mAbs of known epitope specificity using gp41 ectodomain ZA1197. Binding of biotinylated variants (FIG. 7A) QA255.006 and (FIG. 7B) QA255.016 to gp41 ectodomain protein ZA1197. Binding was assessed in competition with the panel of mAbs with defined epitope specificity indicated on the x-axis.

FIG. 8A, 8B. Phage Immunoprecipitation-Sequencing (PhIP-Seq) can be performed on a known HIV antibody (e.g., 240-D which targets gp41). The enriched sequences, or sequences that are more common in the sequences immunoprecipitated condition compared to the original library or mock treated library, are indicated by the circle (FIG. 8A). Fold enrichment over input shows that the enriched peptides map to a specific region of HIV gp41 (FIG. 8B).

FIGS. 9A-9C. Peptides enriched in phage display immunoprecipitation with gp41 mAbs and their variation in natural sequences. HIV HXB2 is the reference sequence. (FIG. 9A) HIV envelope (Env) sequences are listed under the reference sequence. The peptides that were enriched by phage display immunoprecipitation for QA255.067 (SEQ ID NOs: 1-20) and QA255.072 (SEQ ID NOs: 1-16, 18, 21, and 22) are shown, with the mostly highly enriched peptides shown at the top of the list. The common sequences among all the enriched peptides are underlined. (FIG. 9B) A summary of the core sequences identified for these mAbs (SEQ ID NOs: 1 and 23-25) compared to 240-D is shown. (FIG. 9C) Logo plot of 5,471 sequences of HIV from the LANL database across the epitopes defined for these mAbs.

FIG. 10. Alignment of QA255 HIV Env sequences to evaluate gp41-mAb-specific escape (SEQ ID NOs: 27-44). Alignment of the ectodomain of gp41 for 28 QA255 homologous Env amino acid sequences that include the fusion peptide, N terminal heptad repeat (NHR), and C terminal heptad repeat (CHR). The epitope of QA255.067 and QA255.072 defined in FIGS. 9A-9C and the epitope of mAbs that competed with QA255.006 and QA255.016 (5F3, 167-D; FIGS. 5A-5E) are marked, as are the fusion peptide, NHR and CHR.

FIGS. 11A-11D. Infected cell recognition and ADCC susceptibility to Cluster I and Cluster II antibodies. (FIG. 11A and FIG. 11C) Binding or ADCC activity (FIG. 11B and FIG. 11D) was measured against cells infected with a wildtype NL4.3 virus construct expressing the ADA envelope (pNL43/ADA/WT), the construct with a deficient nef (pNL43/ADA/N-) or vpu gene (pNL43/ADA/U-), the construct with both nef- and vpu-deficient genes (pNL43/ADA/N-U-), or the construct with both deficient genes and containing the D368R mutation in the ADA envelope. In FIG. 11C and FIG. 11D, cells were treated with IFNα as described in Example 1. Data represents the average+/−standard deviation (SD) of 5 (FIG. 11A and FIG. 11B) and 4 (FIG. 11C and FIG. 11D) independent experiments.

FIGS. 12A, 12B. Cell based ELISA to detect Env recognition at the cell surface. (FIG. 12A) Binding to 293T cells transfected with an empty pcDNA3.1 plasmid or increasing concentrations of a plasmid expressing HIV-1_{JRFL}ΔCT Env as described in Example 1. The key to the antibody tested is shown to the right. (FIG. 12B) Binding to cells pre-incubated in the presence or absence of sCD4 (10 μg/mL for 1 hour (h) at room temperature) before addition of the different anti-Env Abs. The concentration of plasmid expressing HIV Env used in the transfection corresponds to the 1× condition in FIG. 12A. Signals obtained with the empty pcDNA3.1 plasmid (negative control) were subtracted from signals obtained from Env-transfected cells for experiments in FIGS. 12A, 12B. Results are presented as the average+/−SD of relative luminescence units (RLU). Results are representative from three independent experiments performed in quadruplicate.

FIGS. 13A-13D. Phage-DMS: Schematic of the approach to epitope mapping using a deep mutational scanning (DMS) phage display library. (FIG. 13A) To interrogate the role of each amino acid in protein-protein binding interactions, a library of tiled peptides from the protein(s) of interest is generated. After the library of sequences are created, nucleotides encoding the viral proteins are cloned into T7 phage so that the phage expressed the DMS peptides. Phage expressing the DMS peptides are incubated with a monoclonal antibody (mAb) (or binding protein of interest) and antibody-bound phage are isolated using immunoprecipitation. Isolated phage are lysed and deep sequenced to identify the enriched sequences through computational analyses. (FIG. 13B) The results of a Phage-DMS experiment will show enriched sequences and non-enriched sequences. The box shows the Epitope region. Enriched peptides spanning the epitope region have mutations that tolerate the epitope, whereas peptides spanning the epitope region that are not enriched, have mutations that disrupt the epitope and result in a loss of binding. FIG. 13C shows a hypothetical example of mutations (underlined) in enriched peptide sequences (SEQ ID NOs: 47-49) that are tolerated/do not disrupt the epitope. A known epitope for 240D is shown in SEQ ID NO: 45. FIG. 13D shows representative mutations (italicized and underlined) in non-enriched peptide sequences (SEQ ID NOs: 50-53) that disrupt the epitopes and allow escape. The epitope region is boxed.

FIGS. 14A-14G. DMS/phage defines the linear epitopes of gp41-specific antibodies and a positive control antibody (240D). In this experiment, a library of peptides spanning 3 gp41 sequences was included: BF520.W14.C2, BG505.W6.C2, and ZA1197. In this library, the peptides were 31 amino acids in length with either a wildtype (not underlined) or mutant residue (exemplary mutant residues are underlined) at the central amino acid, across the ectodomain of HIV gp41 (FIG. 14A). The DMS phage display library sampled every possible single-amino acid in the gp41 ectodomain. A Phage-DMS experiment is setup with newly identified gp41 mAbs and a positive control (240D) with a defined gp41 epitope (FIG. 14B). The scaled differential selection values are displayed for the control mAb 240D, with the wildtype (WT) amino acid at 0 on the y-axis and mutant amino acids either above or below WT (FIG. 14C). The positive control mAb 240D bound to peptides with the expected amino acids as defined by prior studies (FIG. 14C). QA255.006 (FIG. 14D) and QA255.016 (FIG. 14E) did not significantly enrich for any peptides above background. Both QA255.067 (FIG. 14F) and QA255.072 (FIG. 14G) significantly enriched for peptides spanning the immunodominant C-C loop region of gp41, with certain mutations in this region abolishing mAb binding, indicating they disrupt a residue critical to the epitope.

FIG. 15. Phage-DMS reveals sites of binding between gp41 peptides from HIV strain BG505 and mAb 240D. Phage-DMS results are displayed in heatmap form across amino acid positions 580-610. The wild type amino acid in BG505 is indicated with the amino acid number at the bottom of each column and these are also shown as dots in the figure. The rows show the results for amino acid residue at each of the positions, grouped by the characteristics of the amino acid. Mutations to sites resulting in a loss of binding relative to WT have a white triangle in the box and sites that result in increased binding have a white four-point star in the box. For example, G594, C595, and L599 more often demonstrate mutations to sites resulting in a loss of binding relative to WT and L589 more often shows binding sites that result in increased binding. These results are consistent with the known epitope of 240D; for example, the C at position 595 is critical to the epitope and all changes to that position decrease binding. The G at position 594 and the L at position 599 are also preferred amino acids for the 240D mAb.

FIGS. 16A-16E. Results of antibody binding assays by an ELISA for various peptide variants that were predicted to have altered binding by Phage-DMS. Select mutant peptides predicted by Phage-DMS to either increase or decrease binding to gp41 mAbs and V3 mAbs were synthesized and are shown in FIG. 16A. The strain of the HIV in the Phage-DMS library that these variants are based on is indicated along with the amino acid positions with the protein based on standard HIV HXB2 numbering. These peptides were tested in a peptide competition ELISA: gp41 peptides were preincubated with the gp41-specific antibodies 240D (FIG. 16B) and F240 (FIG. 16C) and V3 peptides were preincubated with the V3-specific antibodies 447-52D (FIG. 16D) and 257D (FIG. 16E) before performing an ELISA. An IC50 value was calculated for each peptide to quantify the effect of each mutation on antibody binding. An IC50 that is higher than the wildtype suggest that the amino acid variant binds better to the mAb than wildtype whereas a lower IC50 indicates the amino acid variant leads to reduced binding. * indicate statistically significant differences. The results include three different experiments.

FIGS. 17A, 17B. Correlation between Phage-DMS results and the competition ELISA. Scaled differential selection values as determined by Phage-DMS were correlated with the IC50 value determined by competition peptide ELISA for each mutation examined in the ELISA. Results with gp41-specific antibodies (FIG. 17A) and V3-specific antibodies (FIG. 17B) are shown. The Pearson correlation coefficient along with the p-value is displayed.

### DETAILED DESCRIPTION

Proteins are made of strings of amino acids with different proteins having different numbers and orders of amino acids. Proteins are essential to the functioning of cells and organisms. A powerful way to study proteins is through mutagenesis. Mutagenesis refers to altering the amino acid that naturally occurs at a position along the string of amino acids that create a given protein. Systematically altering amino acids at different positions through mutagenesis can identify those amino acids that are essential to the function of the protein. Deep mutational scanning (DMS) refers to methods of generating and characterizing hundreds of thousands of mutants or more of a given protein of interest. More particularly, DMS can refer to altering each amino acid position with all possible alternative amino acids of a given protein of interest and assessing the effects of each individual substitution. DMS can also be used to interrogate a subset of mutations, at select residues or with a subset of possible amino acids in the same manner. DMS can be used to define the residues of a candidate binding molecule that are essential for interaction with a protein of interest, for example, antibody binding to a viral protein or protein ligand binding to a cellular receptor or the converse.

One scenario where the study of proteins is extremely beneficial is in relation to viruses and antibody binding to proteins located on viruses. The first step in viral infection is binding of a viral entry protein to a host cell. Viral entry proteins are a primary target of immune system responses against infection. To combat the spread of viruses, such as influenza, human immunodeficiency virus (HIV), Ebola virus, Zika virus, and coronavirus (CoV), to name a few, scientists and doctors need tools to know when antibodies are working against viral proteins (e.g., viral entry proteins), or conversely, when these viral proteins have developed resistance to therapeutics and pose a greater risk. For this, they often need to know how these antibodies interact with and bind different viral proteins and which amino acid interactions are critical for binding.

DMS has been applied for this purpose, however previous approaches raise a number of logistical and safety concerns. For example, in previous experimental work, antibody epitopes and pathways of antibody escape for HIV were assessed. In these studies, mutations were introduced into the viral protein of interest (HIV Env) and the resulting library of viruses were tested against specific target antibodies using functional assays (e.g., neutralization assays). This approach was successful but was limited in the number of experiments that could be done because of the need for large amounts of infectious virus for each experiment. This approach also required a functional assay for neutralization and thus was not amenable to the study of antibodies that have other functions.

Bacteriophage (commonly referred to as phage) are viruses that infect and replicate within bacteria. Phage are not infectious for humans or other animals. Researchers have used phage display libraries to study protein:protein interactions. Historically, phage display relied on the production of very large collections of random peptides associated with their corresponding genetic blueprints (Scott et al., Science 249:386-390 (1990); Dower, Curr Biol 2:251-253 (1992); Cortese et al., Trends Biotechnol 12:262-267 (1994); Cortese et al., Curr Opin Biotechnol 7:616-621 (1996)). Presentation of the random peptides was often accomplished by constructing chimeric proteins expressed on the outer surface of the phage. This presentation made the libraries amenable to the study of binding assays in the form of biopanning (Parmley et al., Gene 73:305-318 (1988)) leading to the affinity isolation and identification of peptides with desired binding properties.

Currently, phage display enables the expression of designed proteins and peptides on the surface of phage particles, with a direct link between the genotype and the phenotype of the peptide or protein of interest. This method enables vast libraries of peptides or proteins to be screened simultaneously for their ability to interact with candidate binding molecules, such as ligands, enzyme substrates, antibodies and the like.

Despite independent advancements in the use of DMS and in the use of phage display libraries over the last decades, there is still significant room for improvement in the ability to precisely identify amino acid residues within a protein that are essential for the protein's binding to other molecules.

The current disclosure provides protein residue mapping using a combination of deep mutational scanning (DMS) of a protein of interest expressed by a phage library; exposure of the phage library to a potential binding molecule; an efficient immunoprecipitation step to isolate bound complexes of proteins of interest and the binding molecule; and identifying the sequences of bound peptides to perform protein residue mapping. The combined process is referred to herein as Phage-DMS.

In particular embodiments, the efficient immunoprecipitation step includes PhIP-Seq (see, e.g., Mohan et al., Nature Protocols, 13, 1958-1978 (2018); Williams, et al., PLoS Pathog, 15(2): e1007572, 2019). PhIP-Seq has been most commonly applied to the detection of autoantibodies by probing sera from peptides that cross react with the human proteome (Larman, Nature Biotech, 29:535, 2011). PhIP-Seq has also been used to screen sera for antibodies to viral infections in a method called VirScan (Xu, Science, 348:1105, 2015). In VirScan, the phage library contains the proteome of a large collection of viruses and using this method, prior viral infections can be detected based on the antibody profile. PhIP-Seq has also been used for antibody mapping of monoclonal antibodies (Williams et al. PLoS Pathog 15(2): e1007572, 2019). In this approach, a custom-made phage library that encoded the proteome of multiple viruses was used to map the epitope of HIV-specific monoclonal antibodies. Before the current disclosure, however, DMS of phage libraries had not been used in combination with PhIP-Seq.

The current disclosure's combination of a phage DMS library with PhIP-Seq results in a method that uses phage to display a collection of DMS peptides of a protein of interest. Following exposure to a candidate binding molecule, bound DMS-peptide expression phage/candidate binding molecule complexes are isolated using immunoprecipitation. Once bound and unbound DMS-peptide expressing phage are separated, one or both groups can be deep sequenced allowing the mapping of protein residues that are critical to the interaction between the DMS peptides and the candidate binding molecule. This method is high-throughput and can be conducted in a single tube or well once the DMS peptide and associated phage library is generated. Further, the phage library can be used repeatedly by re-growing and amplifying the phage expressing the DMS peptides.

The combination methods disclosed herein vastly simplify previously used approaches to protein residue mapping and overcome numerous bottlenecks and safety considerations associated with currently available protein residue mapping processes. For example, as indicated, the currently disclosed methods can include an entire DMS library for a protein of interest in one experimental tube. Additionally, the use of molecular barcodes is not needed. In this context, a barcode (Hiatt et al., Nat Methods 7:119-122, 2010) refers to a random stretch of nucleotides that serves as a unique tag to identify a DNA molecule that is sequenced. Previously, each variant in a library of sequences was associated with such barcode to help in distinguishing true mutations from sequencing errors.

Further, and as previously indicated, the currently disclosed methods do not rely on the use of functional assays to determine presence or absence of protein interaction. Functional assays can include any assay that detects interaction of a protein of interest to a candidate binding molecule by measuring the effect of the candidate binding molecule on the protein of interest in a biological process and not by detecting binding directly between the peptide of interest and the candidate binding molecule. For example, in the context of functional assays for antibodies that target viral entry proteins, the ability of a target antibody to neutralize a virus or kill cells infected with a virus can be measured through assays such as plaque reduction assay, microscopic cytopathic effect assay, hemagglutination inhibition assay, neuraminidase inhibition assay, ELISA-based endpoint assessment microneutralization assay, virucidal assay, and virus yield reduction assay. Such assays are not needed within the currently disclosed methods.

The use of DMS with a phage library also circumvents the need for large amounts of virus. Phage also grow to much higher titers and they are also not infectious to humans. These are additional benefits of the disclosed methods.

Aspects of the current disclosure are now described in more supporting detail as follows: (i) Proteins of Interest and Deep Mutational Scanning (DMS) Peptide Libraries; (ii) Phage Libraries; (iii) Candidate Binding Molecules; (iv) Screening and Isolation of Phage/Candidate Binding Molecule Complexes; (v) Nucleotide Processing, Sequencing, and Analysis; (vi) Exemplary Embodiments; (vii) Experimental Examples; and (viii) Closing Paragraphs.

(i) Proteins of Interest and Deep Mutational Scanning (DMS) Peptide Libraries. DMS can be used to measure the functional effects of amino acid mutations in a protein of interest. The protein of interest can be any protein undergoing an analysis of interest. Exemplary proteins of interest are derived from viruses, bacteria, fungus, and/or specific cell types or cancer cells. In particular embodiments, a peptide or protein can refer to one or more regions or domains of a protein of interest.

In particular embodiments, the protein of interest is a viral protein. In particular embodiments, the viral protein is a human immunodeficiency virus-1 (HIV-1) viral protein, an HIV-2 viral protein, a simian immunodeficiency virus (SIV) viral protein, an influenza virus viral protein, an Ebola virus viral protein, a coronavirus (CoV) viral protein, a Wuhan CoV (COVID) viral protein, a severe acute respiratory syndrome CoV (SARS-CoV) viral protein, a Middle East respiratory syndrome CoV (MERS-CoV) viral protein, a Lassa virus viral protein, a Nipah virus viral protein, a Chikungunya virus viral protein, a Hendra virus viral protein, a hepatitis B virus viral protein, a hepatitis C virus viral protein, a measles virus viral protein, a Rabies virus viral protein, a respiratory syncytial virus (RSV) viral protein, a Zika virus viral protein, a Dengue virus viral protein, or a Herpes virus viral protein.

Viral proteins of interest include viral entry proteins. Examples of viral entry proteins include [virus (entry protein)]: Chikungunya (E1 Env and E2 Env); Ebola glycoprotein (EBOV GP); Hendra (F glycoprotein and G glycoprotein); hepatitis B (large (L), middle (M), and small (S)); hepatitis C (glycoprotein E1 and glycoprotein E2); HIV envelope (Env); influenza hemagglutinin (HA); Lassa virus envelope glycoprotein (GPC); measles (hemagglutinin glycoprotein (H) and fusion glycoprotein F0 (F)); MERS-CoV (Spike (S)); Nipah (fusion glycoprotein F0 (F) and glycoprotein G); Rabies virus glycoprotein (RABV G); RSV (fusion glycoprotein F0 (F) and glycoprotein G); and SARS-CoV (Spike (S)); among many others.

Additional HIV proteins include gene products of the gag, pol, and env genes such as HIV gp32, HIV gp41, HIV gp120, HIV gp160, HIV P17/24, HIV P24, HIV P55 GAG, HIV P66 POL, and HIV GP36. Other HIV proteins of interest include the Nef protein and other accessory proteins such as Vpr, Vpu, Tat, and Rev. Very particular examples of specific viral proteins and strains include BF520.W14.C2; BG505.W6M.C2.T332N; BG505 SOSIP Env trimer; BL035.W6M.ENV.C1; SF162; ZM109F.PB4; C2-94UG114; HIV-BAL, HIV-LAI, SIV/mac239; MN gp41 monomer; ectodomain ZA.1197/MB; Q23; QA013.70I.Env.H1; QA013.385M.Env.R3 677; QB850.73P.C14; QB850.632P.B10; Q461.D1; and QC406.F3. Numerous additional proteins/strains are known to one of ordinary skill in the art.

As further particular examples of viral proteins of interest, cytomegaloviral antigens include envelope glycoprotein B and CMV pp65; Epstein-Barr antigens include EBV EBNAI, EBV P18, and EBV P23; hepatitis antigens include the S, M, and L proteins of hepatitis B virus, the pre-S antigen of hepatitis B virus, HBCAG DELTA, HBV HBE, hepatitis C viral RNA, HCV NS3 and HCV NS4; herpes simplex viral antigens include immediate early proteins and glycoprotein D; influenza antigens include hemagglutinin and neuraminidase; Japanese encephalitis viral antigens include proteins E, M-E, M-E-NS1, NS1, NS1-NS2A and 80% E; measles antigens include the measles virus fusion protein; rabies antigens include rabies glycoprotein and rabies nucleoprotein; respiratory syncytial viral antigens include the RSV fusion protein and the M2 protein; rotaviral antigens include VP7sc; rubella antigens include proteins E1 and E2; and varicella zoster viral antigens include gpl and gpll.

As indicated, in addition to viral proteins, proteins of interest can include bacterial proteins, fungal proteins, and cancer antigens.

Bacterial proteins of interest can be derived from, for example, anthrax, gram-negative bacilli, chlamydia, diptheria, Helicobacter pylori, Mycobacterium tuberculosis, pertussis toxin, pneumococcus, rickettsiae, staphylococcus, streptococcus and tetanus.

As particular examples of bacterial antigen markers, anthrax antigens include anthrax protective antigen; gram-negative bacilli antigens include lipopolysaccharides; diptheria antigens include diptheria toxin; Mycobacterium tuberculosis antigens include mycolic acid, heat shock protein 65 (HSP65), the 30 kDa major secreted protein and antigen 85A; pertussis toxin antigens include hemagglutinin, pertactin, FIM2, FIM3 and adenylate cyclase; pneumococcal antigens include pneumolysin and pneumococcal capsular polysaccharides; rickettsiae antigens include rompA; streptococcal antigens include M proteins; and tetanus antigens include tetanus toxin.

Fungal proteins of interest can be derived from, for example, candida, coccidiodes, cryptococcus, histoplasma, leishmania, plasmodium, protozoa, parasites, schistosomae, tinea, toxoplasma, and Trypanosoma cruzi.

As particular examples of fungal antigens, coccidiodes antigens include spherule antigens; cryptococcal antigens include capsular polysaccharides; histoplasma antigens include heat shock protein 60 (HSP60); leishmania antigens include gp63 and lipophosphoglycan; plasmodium falciparum antigens include merozoite surface antigens, sporozoite surface antigens, circumsporozoite antigens, gametocyte/gamete surface antigens, protozoal and other parasitic antigens including the blood-stage antigen pf 155/RESA; schistosomae antigens include glutathione-S-transferase and paramyosin; tinea fungal antigens include trichophytin; toxoplasma antigens include SAG-1 and p30; and Trypanosoma cruzi antigens include the 75-77 kDa antigen and the 56 kDa antigen.

Cancer antigen proteins of interest can be derived from, for example, brain cancer, breast cancer, colon cancer, HBV-induced hepatocellular carcinoma, intestinal cancer, kidney cancer, leukemia, liver cancer, lung cancer, lymphoma, melanoma, mesothelioma, multiple myeloma, ovarian cancer, pancreatic cancer, prostate cancer, renal cell carcinoma, stem cell cancer, stomach cancer, throat cancer, or uterine cancer.

Particular antigen markers associated with cancers cells include A33, β-catenin, BAGE, Bcl-2, BCMA, c-Met, CA19-9, CA125, CAIX, CD5, CD19, CD20, CD21, CD22, CD24, CD33, CD37, CD45, CD123, CD133, CEA, CS-1, cyclin B1, DAGE, EBNA, EGFR, ephrinB2, ERBB2, estrogen receptor, FAP, ferritin, folate-binding protein, GAGE, G250, GD2, GM2, gp75, gp100 (Pmel 17), HER-2/neu, HPV E6, HPV E7, Ki-67, LRP, mesothelin, p53, PRAME, progesterone receptor, PSA, PSCA, PSMA, MAGE, MART, mesothelin, MUC, MUM-1-B, myc, NYESO-1, ras, RORI, SV40 T, survivin, tenascin, TSTA tyrosinase, VEGF, and WT1.

Proteins of interest are converted into DMS peptides to create a DMS library. In particular embodiments, a DMS library can express a full protein of interest. In particular embodiments, DMS peptides include tiled or staggered overlapping segments of the protein of interest. In particular embodiments, DMS peptides are selected to have a length to allow efficient and accurate sequencing. As long as a synthesis technique is available, proteins and/or their fragments can be any length. In particular embodiments, a protein can be broken into peptide fragments of 10-40, 20-50 amino acids, 30-80 amino acids, 50-150 amino acids, 100-300 amino acids, 150-500 amino acids, or greater. In particular embodiments, a protein can be broken into peptide fragments of 28-32 amino acids, or 28, 29, 30, 31, 32, 33, 34, 35, 36, 37, 38, 39, 40, 41, 42, 43, 44, 45, 46, 47, 48, 49, or 50 amino acids. In particular embodiments, a peptide or protein can be of length sufficient to map discontinuous epitopes in a protein of interest.

In particular embodiments, overlapping fragments of a protein of interest are generated by moving one amino acid residue position down the length of the protein while maintaining the same length of peptide fragments. In particular embodiments, each staggered overlapping fragment can include a single amino acid mutation. The single amino acid mutation can be located at the center position of the DMS peptide. While these described approaches are preferable, embodiments that stagger DMS peptide fragments by an integer greater than 1 (e.g., 2, 3, 4, or 5) and/or place a mutation position outside of the center position of a DMS peptide may also be used. These methods could also be used to introduce multiple mutations into the same peptide to study their combined effects on binding.

In particular embodiments, a DMS library includes a complete set of possible protein variants of a protein of interest, with 19 possible amino acid substitutions at each amino acid position. These embodiments can also include all possible codons of the associated 63 codons at each amino acid position. It could also include a subset of amino acid variants at each position as discussed below. In particular embodiments, a DMS library includes a complete set of possible protein variants of a protein of interest, with 19 possible amino acid substitutions at each amino acid position but with less than all possible encoding codons. In particular embodiments, a DMS library includes or encodes all possible amino acids at all positions of a protein of interest, and each variant protein is encoded by more than one variant nucleotide sequence. In particular embodiments, a DMS library includes or encodes all possible amino acids at all positions of a protein of interest, and each variant protein is encoded by one nucleotide sequence.

In particular embodiments, a DMS library includes or encodes all possible amino acids at less than all positions of a protein, for example at 10%, 20%, 30%, 40%, 50%, 60%, 70%, 80%, 90%, 95%, 96%, 97%, 98% or 99% of positions. In particular embodiments, a DMS library includes or encodes less than all possible amino acids (for example 10%, 20%, 30%, 40%, 50%, 60%, 70%, 80%, 90%, 95%, 96%, 97%, 98% or 99% of potential amino acids) at all positions of a protein. In particular embodiments, a DMS library includes or encodes less than all possible amino acids (for example 10%, 20%, 30%, 40%, 50%, 60%, 70%, 80%, 90%, 95%, 96%, 97%, 98% or 99% of potential amino acids) at less than all positions of a protein, for example at 10%, 20%, 30%, 40%, 50%, 60%, 70%, 80%, 90%, 95%, 96%, 97%, 98% or 99% of positions. While these embodiments can be practiced, embodiments with all possible amino acid substitutions at all possible positions are preferred.

In particular embodiments, a DMS library can be synthetically constructed by and/or obtained from a synthetic DNA company such as Twist Bioscience (San Francisco, Calif.). In particular embodiments, methods to generate a codon-DMS library include: polymerase chain reaction (PCR) mutagenesis (Dingens et al. Cell Host and Microbe. 2017; 21(6):777-787; Dingens et al. Immunity. 2019 Jan. 29); nicking mutagenesis as described in Wrenbeck et al. (Nature Methods 13: 928-930, 2016) and Wrenbeck et al. (Protocol Exchange doi:10.1038/protex.2016.061, 2016); PFunkel (Firnberg & Ostermeier, PLoS ONE 7(12): e52031, 2012); massively parallel single-amino-acid mutagenesis using microarray-programmed oligonucleotides (Kitzman et al., Nature Methods 12: 203-206, 2015); and saturation editing of genomic regions with CRISPR-Cas9 (Findlay et al., Nature 513(7516): 120-123, 2014). Mutagenesis methods that give a larger proportion of single amino acid mutants are known in the art (see, e.g., Kitzman, et al., Nature Methods 12: 203-206, 2015; Firnberg & Ostermeier, PLoS One 7: e52031, 2012; Jain & Varadarajan, Anal. Biochem. 449: 90-98, 2014; and Wrenbeck et al., Nature Methods 13: 928, 2016).

Sequences encoding DMS peptides result in DMS peptides expressed by phage. In particular embodiments, expressed DMS peptides that form a DMS library can include functional sequences, such as transport sequences, buffer sequences, tags, and/or selectable markers so long as the functional sequence does not interfere with binding between the DMS peptides and a potential candidate binding molecule or bind to the candidate itself.

Transport sequences facilitate display of DMS peptides on the surface of the phage expressing the peptide. In particular embodiments, transport sequences include any protein normally found at the surface of a phage, such as a filamentous phage (e.g., phage f1, fd, and M13) or a bacteriophage (e.g., λ, T4 and T7) that can be adapted to be expressed as a fusion protein with a DMS peptide and still be assembled into a phage particle such that the DMS peptide is displayed on the surface of the phage. Suitable surface proteins derived from filamentous phage include minor coat proteins, such as gene Ill proteins and gene VIII proteins; and major coat proteins such as, gene VI proteins, gene VII proteins, and gene IX proteins. Suitable surface proteins derived from bacteriophage include gene 10 proteins from T7 and capsid D protein (gpD) from bacteriophage A. In particular embodiments, a suitable transport sequence is a domain, a truncated version, a fragment, or a functional variant of a naturally occurring surface protein. For example, a suitable transport sequence can be a domain of the gene Ill protein, e.g., the anchor domain or “stump.” Additional exemplary phage surface proteins that can be used as transport sequences are described in WO 00/71694. As appreciated by the skilled artisan, the choice of a transport sequence can to be made in combination with a consideration of the phage used within the phage library. Exemplary leader sequences for DMS peptides include a PelB leader sequence and/or an OmpA leader sequence.

Buffer sequences can be used to present the residue mutated within a peptide at a common position within the peptides of a DMS library. To facilitate this, DMS peptides can include buffer sequences, that is residues that allow placement of the mutated residue at the common position. In particular embodiments, the length of the buffer sequence will be dependent on the position of the mutated residue within the reference wild-type protein. In particular embodiments, the buffer sequence includes a (Gly₄Ser)₃ sequence (GGGGSGGGGSGGGGS, SEQ ID NO: 74) as described in Klein et al. (Protein Eng Des Sel. 27(10): 325-330, 2014). In particular embodiments, the buffer sequence can include (Gly)ₙ, where n=1 to 10 (e.g., n=1, 2, 3, 4, 5, 6, 7, 8, 9, or 10; SEQ ID NO: 75); (Ser)ₙ, where n=1 to 10 (e.g., n=1, 2, 3, 4, 5, 6, 7, 8, 9, or 10; SEQ ID NO: 76), (Ala)ₙ, where n=1 to 10 (e.g., n=1, 2, 3, 4, 5, 6, 7, 8, 9, or 10; SEQ ID NO: 77), (Gly-Ser)ₙ, where n=1 to 10 (e.g., n=1, 2, 3, 4, 5, 6, 7, 8, 9, or 10; SEQ ID NO: 78), (Gly-Ser-Ser-Gly)ₙ, where n=1 to 10 (e.g., n=1, 2, 3, 4, 5, 6, 7, 8, 9, or 10; SEQ ID NO: 79), (Gly-Ser-Gly)ₙ, where n=1 to 10 (e.g., n=1, 2, 3, 4, 5, 6, 7, 8, 9, or 10; SEQ ID NO: 80), (Gly-Ser-Ser)ₙ, where n=1 to 10 (e.g., n=1, 2, 3, 4, 5, 6, 7, 8, 9, or 10; SEQ ID NO: 81), (Gly-Ala)ₙ, where n=1 to 10 (e.g., n=1, 2, 3, 4, 5, 6, 7, 8, 9, or 10; SEQ ID NO: 82), or any combination thereof.

In addition, DMS peptides can optionally include a tag that may be useful in purification, detection and/or screening. Suitable tags include, for example, His tag (HHHHHH; SEQ ID NO: 66), Flag tag (DYKDDDDK; SEQ ID NO: 67), Xpress tag (DLYDDDDK; SEQ ID NO: 68), Avi tag (GLNDIFEAQKIEWHE; SEQ ID NO: 69), Calmodulin tag (KRRWKKNFIAVSAANRFKKISSSGAL; SEQ ID NO: 70), Polyglutamate tag, HA tag (YPYDVPDYA; SEQ ID NO: 71), Myc tag (EQKLISEEDL; SEQ ID NO:72), Strep tag (which refers the original STREP® tag (WRHPQFGG; SEQ ID NO: 73), STREP® tag II (WSHPQFEK SEQ ID NO:83 (IBA Institut fur Bioanalytik, Germany); see, e.g., U.S. Pat. No. 7,981,632), Softag 1 (SLAELLNAGLGGS; SEQ ID NO: 84), Softag 3 (TQDPSRVG; SEQ ID NO: 85), V5 tag (GKPIPNPLLGLDST; SEQ ID NO: 86) a gD-tag, a c-myc tag, green fluorescence protein tag, a GST-tag or β-galactosidase tag. Tags can also include detectable labels. Detectable labels can include any suitable label or detectable group detectable by, for example, optical, spectroscopic, photochemical, biochemical, immunochemical, electrical, optical or chemical means.

Phage-display vectors can also include a promoter suited for constitutive or inducible expression. Examples of inducible promoters include the lac promoter, the lac UV5 promoter, the arabinose promoter, and the tet promoter. In particular embodiments, an inducible promoter can be further restricted by incorporating repressors (e.g., lacI) or terminators (e.g., a tHP terminator). For example, repressor lacI can be used together with the Lac promoter.

Phage-display vectors can also include other useful components such as ribosome binding sites; restriction sites; termination codons; insulator and/or post-regulatory elements; etc.

In general, a phage-display vector includes a promoter and/or other regulatory regions operably linked to the polynucleotide sequence encoding the DMS peptide (and other selected functional sequences). The term “operably linked” refers to a functional linkage between nucleic acid sequences such that the linked promoter and/or regulatory region functionally controls expression of the coding sequence. It also refers to the linkage between coding sequences such that they may be controlled by the same promoter and/or regulatory region. Such linkage between coding sequences may also be referred to as being linked in-frame or in the same coding frame such that a fusion protein including the amino acids encoded by the coding sequences may be expressed.

(ii) Phage-DMS Libraries. A Phage-DMS library is a library of phage (also referred to as a phage library) expressing and displaying a DMS library of a protein of interest on the outside of the phage virion. In particular embodiments, a phage-display vector used to generate a Phage-DMS library is a vector including polynucleotide sequences capable of expressing, or conditionally expressing, a heterologous peptide (such as a DMS peptide), for example, as a fusion protein with a phage protein (e.g., a transport sequence). In particular embodiments, a phage-display vector is derived from a filamentous phage (e.g., phage f1, fd, and M13) or a bacteriophage (e.g., T7 bacteriophage, T4 phage, or a lambdoid phage). Filamentous phage and bacteriophage are described in, e.g., Santini (J. Mol. Biol. 282:125-135, 1998), Rosenberg et al. (Innovations 6:1-6, 1996), and Houshmand et al. (Anal. Biochem. 268:363-370, 1999).

Methods for constructing phage-display vectors, phage-display libraries and associated methods of use are described in, for example, U.S. Pat. No. 5,223,409; Smith (1985) Science 228:1315-1317; WO 92/18619; WO 91/17271; WO 92/20791; WO 92/15679; WO 93/01288; WO 92/01047; WO 92/09690; WO 90/02809; de Haard, et al. (1999) J. Biol. Chem. 274:18218-30; Hoogenboom, et al. (1998) Immunotechnoloqy 4:1-20; Hoogenboom, et al. (2000) Immunol, Today 2:371-8; Fuchs, et al. (1991) Bio/Technology 9:1370-1372; Huse, et al. (1989) Science 246:1275-1281; Griffiths, et al. (1993) EMBO J. 12:725-734; Hawkins, et al. (1992) J. Mol. Biol. 226:889-896; Clackson, et al. (1991) Nature 352:624-628; Gram, et al. (1992) PNAS 89:3576-3580; Garrard, et al. (1991) Bio/Technology 9:1373-1377; Rebar, et al. (1996) Methods Enzymol. 267:129-49; Hoogenboom, et al. (1991) Nucl. Acid Res. 19:4133-4137; and Barbas, et al. (1991) PNAS 88:7978-7982.

In particular embodiments, a phage library is validated to confirm that each intended DMS protein or peptide is expressed by the library. A library can be considered validated when, following at least 50%, at least 60%, at least 70%, at least 80%, at least 90%, at least 95%, at least 98% or 100% of the intended DMS proteins or peptides are identified as expressed by the phage library.

In particular embodiments, validation can include deep sequencing of nucleotides cloned into the phage library; and analysis of the resulting sequences as compared to the original sequences cloned into the library to determine how well they correspond. Validation can also include performing Sanger sequencing on individual plaques grown up from the library to confirm fidelity of the library. Results of validation analyses can be used to provide a baseline against which results can be compared.

In particular embodiments, a library is considered validated when, following a validation analysis, at least 90% of the clones in the library have a clonal frequency within one log of each other and/or if at least 90% of the phage library has a sequencing depth of at least 10 reads per clone. Herein, clonal frequency refers to the relative frequency of clones in a cell population that arose by equal proliferation by all cells in the population. Sequencing depth refers to the number of unique reads that include a given nucleotide in the sequence. A sequencing depth can be determined at each residue in a sequence. A read refers to each inferred sequence of base pairs corresponding to a portion of the DNA being sequenced.

Particular embodiments can utilize selectable markers during library generation. For example, amp resistance can be used to select for bacteria which serve as a host for the bacteriophage. Other examples of selectable markers include cerulenin resistance genes (e.g., fas2m, PDR4; Inokoshi et al., Biochemistry 64: 660, 1992; Hussain et al., Gene 101: 149, 1991); copper resistance genes (CUP1; Marin et al., Proc. Natl. Acad. Sci. USA. 81: 337, 1984); and geneticin resistance gene (G418r) as markers.

It can also be appropriate to use auxotrophic markers as reporters. Exemplary auxotrophic markers include methionine auxotrophic markers (e.g., met1, met2, met3, met4, met5, met6, met7, met8, met10, met13, met14 or met20); tyrosine auxotrophic markers (e.g., tyr1 or isoleucine); valine auxotrophic markers (e.g., ilv1, ilv2, ilv3 or ilv5); phenylalanine auxotrophic markers (e.g., pha2); glutamic acid auxotrophic markers (e.g., glu3); threonine auxotrophic markers (e.g., thr1 or thr4); aspartic acid auxotrophic markers (e.g., asp1 or asp5); serine auxotrophic markers (e.g., ser1 or ser2); arginine auxotrophic markers (e.g., arg1, arg3, arg4, arg5, arg8, arg9, arg80, arg81, arg82 or arg84); uracil auxotrophic markers (e.g., ura1, ura2, ura3, ura4, ura5 or ura6); adenine auxotrophic markers (e.g., ade1, ade2, ade3, ade4, ade5, ade6, ade8, ade9, ade12 or ade15); lysine auxotrophic markers (e.g., lys1, lys2, lys4, lys5, lys7, lys9, lys11, lys13 or lys14); tryptophan auxotrophic markers (e.g., trp1, trp2, trp3, trp4 or trp5); leucine auxotrophic markers (e.g., leu1, leu2, leu3, leu4 or leu5); and histidine auxotrophic markers (e.g., his1, his2, his3, his4, his5, his6, his7 or his8).

(iii) Candidate Binding Molecules. Once created, a phage library expressing a DMS library can be exposed to a candidate binding molecule. The candidate binding molecule can be any substance capable of binding a DMS peptide. Exemplary candidate binding molecules include antibodies, ligands, peptides, peptide aptamers, receptors, or combinations and engineered fragments or formats thereof.

Antibodies are produced from two genes, a heavy chain gene and a light chain gene. Generally, an antibody includes two identical copies of a heavy chain, and two identical copies of a light chain. Within a variable heavy chain and variable light chain, segments referred to as complementary determining regions (CDRs) dictate epitope binding. Each heavy chain has three CDRs (i.e., CDRH1, CDRH2, and CDRH3) and each light chain has three CDRs (i.e., CDRL1, CDRL2, and CDRL3). CDR regions are flanked by framework residues (FR).

Antibodies include monoclonal antibodies, human antibodies, humanized antibodies, synthetic antibodies, non-human antibodies, recombinant antibodies, chimeric antibodies, bispecific antibodies, mini bodies, and linear antibodies.

In particular embodiments, the candidate binding molecule includes a humanized antibody. In particular embodiments, a non-human antibody is humanized, where one or more amino acid residues of the antibody are modified to increase similarity to an antibody naturally produced in a human or fragment thereof. These nonhuman amino acid residues are often referred to as “import” residues, which are typically taken from an “import” variable molecule. As provided herein, humanized antibodies or antibody fragments include one or more CDRs from nonhuman immunoglobulin molecules and framework regions wherein the amino acid residues including the framework are derived completely or mostly from human germline. A humanized antibody can be produced using a variety of techniques known in the art, including CDR-grafting (see, e.g., European Patent No. EP 239,400; WO 91/09967; and U.S. Pat. Nos. 5,225,539, 5,530,101, and 5,585,089), veneering or resurfacing (see, e.g., EP 592,106 and EP 519,596; Padlan, 1991, Molecular Immunology, 28(4/5):489-498; Studnicka et al., 1994, Protein Engineering, 7(6):805-814; and Roguska et al., 1994, PNAS, 91:969-973), chain shuffling (see, e.g., U.S. Pat. No. 5,565,332), and techniques disclosed in, e.g., US 2005/0042664, US 2005/0048617, U.S. Pat. Nos. 6,407,213, 5,766,886, WO 9317105, Tan et al., J. Immunol., 169:1119-25 (2002), Caldas et al., Protein Eng., 13(5):353-60 (2000), Morea et al., Methods, 20(3):267-79 (2000), Baca et al., J. Biol. Chem., 272(16): 10678-84 (1997), Roguska et al., Protein Eng., 9(10):895-904 (1996), Couto et al., Cancer Res., 55 (23 Supp):5973s-5977s (1995), Couto et al., Cancer Res., 55(8):1717-22 (1995), Sandhu J S, Gene, 150(2):409-10 (1994), and Pedersen et al., J. Mol. Biol., 235(3):959-73 (1994). Often, framework residues in the framework regions will be substituted with the corresponding residue from the CDR donor antibody to alter, for example improve, target antigen binding. These framework substitutions are identified by methods well-known in the art, e.g., by modeling of the interactions of the CDR and framework residues to identify framework residues important for target antigen binding and sequence comparison to identify unusual framework residues at particular positions. (See, e.g., U.S. Pat. No. 5,585,089; and Riechmann et al., 1988, Nature, 332:323).

In particular embodiments, the antibody binds an HIV viral entry protein. In particular embodiments, the antibody binds gp41. In particular embodiments, the antibody is selected from, QA255.006, QA255.016, QA255.067, and QA255.072. In particular embodiments, the antibody binds gp120. In particular embodiments, the antibody is selected from QA255.105 and QA255.157. Additional exemplary antibodies include VRC01, VRC07, VRC34, PG9, PGT121, PGT145, PGT151, 4E10, 10E8, 10-1074, 50-69, 240-D, 246-D, 5F3, 2F5, 167-D, F240, D5, leronlimab, PRO 542, ibalizumab, b12, PEHRG214, 3BNC117, 131-2G, 12G5, MAB8582, MAB8581, MCA490, 104E5, 38F10, 14G3, 90D3, 56E11, 69F6, c13C6, c2G4, c4G7, c1H3, LCA60, REGN3051, REGN3048, 37.2D, 8.9F, 19.7E, 37.7H, 12.1F, and m102.4.

Additional examples of particular antibodies that can be used as candidate binding molecules include leronlimab (PRO 140), PRO 542, TNX-355 (ibalizumab), anti-RSV G protein monoclonal antibody clone 131-2G, anti-CXCR4 monoclonal antibody clone 12G5 12G5, anti-RSV F protein antibody MAB8582, anti-RSV F protein antibody MAB8581, anti-RSV F protein antibody MCA490, anti-RSV F protein antibody 104E5, anti-RSV F protein antibody 38F10, anti-RSV F protein antibody 14G3, anti-RSV F protein antibody 90D3, anti-RSV F protein antibody 56E11, anti-RSV F protein antibody 69F6, anti-Ebola virus glycoprotein (GP) monoclonal antibody c13C6, anti-Ebola virus glycoprotein (GP) monoclonal antibody c2G4, anti-Ebola virus glycoprotein (GP) monoclonal antibody c4G7, anti-Ebola virus glycoprotein (GP) monoclonal antibody c1H3, LCA60, REGN3051, REGN3048, anti-Lassa virus glycoprotein antibody 37.2D, anti-Lassa virus glycoprotein antibody 8.9F, anti-Lassa virus glycoprotein antibody 19.7E, anti-Lassa virus glycoprotein antibody 37.7H, anti-Lassa virus glycoprotein antibody 12.1F, and Hendra virus neutralizing antibody m102.4. In particular embodiments, the antibody is the influenza-specific mAb Fi6_v3.

Candidate binding molecules can also include binding fragments of antibodies, e.g., Fv, Fab, Fab′, F(ab′)2, and single chain (sc) forms and fragments thereof. In some instances, scFvs can be prepared according to methods known in the art (see, for example, Bird et al., (1988) Science 242:423-426 and Huston et al., (1988) Proc. Natl. Acad. Sci. USA 85:5879-5883). ScFv molecules can be produced by linking VH and VL regions of an antibody together using flexible polypeptide linkers. If a short polypeptide linker is employed (e.g., between 5-10 amino acids) intrachain folding is prevented. Interchain folding is also required to bring the two variable regions together to form a functional epitope binding site. For examples of linker orientations and sizes see, e.g., Hollinger et al. 1993 Proc Natl Acad. Sci. U.S.A. 90:6444-6448, US 2005/0100543, US 2005/0175606, US 2007/0014794, and WO2006/020258 and WO2007/024715. More particularly, linker sequences that are used to connect the VL and VH of an scFv are generally five to 35 amino acids in length. In particular embodiments, a VL-VH linker includes from five to 35, ten to 30 amino acids or from 15 to 25 amino acids. Variation in the linker length may retain or enhance activity, giving rise to superior efficacy in activity studies.

Additional examples of antibody-based candidate binding molecule formats include scFv-based grababodies and soluble VH molecule antibodies. These antibodies form binding regions using only heavy chain variable regions. See, for example, Jespers et al., Nat. Biotechnol. 22:1161, 2004; Cortez-Retamozo et al., Cancer Res. 64:2853, 2004; Baral et al., Nature Med. 12:580, 2006; and Barthelemy et al., J. Biol. Chem. 283:3639, 2008.

In particular embodiments, a VL region in a candidate binding molecule of the present disclosure is derived from or based on a VL of a known monoclonal antibody and contains one or more (e.g., 2, 3, 4, 5, 6, 7, 8, 9, 10) insertions, one or more (e.g., 2, 3, 4, 5, 6, 7, 8, 9, 10) deletions, one or more (e.g., 2, 3, 4, 5, 6, 7, 8, 9, 10) amino acid substitutions (e.g., conservative amino acid substitutions), or a combination of the above-noted changes, when compared with the VL of the known monoclonal antibody. An insertion, deletion or substitution may be anywhere in the VL region, including at the amino- or carboxy-terminus or both ends of this region, provided that each CDR includes zero changes or at most one, two, or three changes and provided a binding molecule containing the modified VL region can still specifically bind its target with an affinity similar to the wild type binding molecule.

In particular embodiments, a binding molecule VH region of the present disclosure can be derived from or based on a VH of a known monoclonal antibody and can contain one or more (e.g., 2, 3, 4, 5, 6, 7, 8, 9, 10) insertions, one or more (e.g., 2, 3, 4, 5, 6, 7, 8, 9, 10) deletions, one or more (e.g., 2, 3, 4, 5, 6, 7, 8, 9, 10) amino acid substitutions (e.g., conservative amino acid substitutions or non-conservative amino acid substitutions), or a combination of the above-noted changes, when compared with the VH of a known monoclonal antibody. An insertion, deletion or substitution may be anywhere in the VH region, including at the amino- or carboxy-terminus or both ends of this region, provided that each CDR includes zero changes or at most one, two, or three changes and provided a binding molecule containing the modified VH region can still specifically bind its target with an affinity similar to the wild type binding molecule.

In particular embodiments, a candidate binding molecule includes or is a sequence that is at least 90%, at least 91%, at least 92%, at least 93%, at least 94%, at least 95%, at least 96%, at least 97%, at least 98%, at least 99%, at least 99.5%, or 100% identical to an amino acid sequence of a light chain variable region (VL) or to a heavy chain variable region (VH), or both, wherein each CDR includes zero changes or at most one, two, or three changes, from a monoclonal antibody or fragment or derivative thereof that specifically binds to a wild-type/reference protein of interest.

An alternative source of candidate binding molecules includes sequences that encode random peptide libraries or sequences that encode an engineered diversity of amino acids in loop regions of alternative non-antibody scaffolds, such as single chain (sc) T-cell receptor (scTCR) (see, e.g., Lake et al., Int. Immunol. 11:745, 1999; Maynard et al., J. Immunol. Methods 306:51, 2005; U.S. Pat. No. 8,361,794), fibrinogen molecules (see, e.g., Weisel et al., Science 230:1388, 1985), Kunitz molecules (see, e.g., U.S. Pat. No. 6,423,498), designed ankyrin repeat proteins (DARPins; Binz et al., J. Mol. Biol. 332:489, 2003 and Binz et al., Nat. Biotechnol. 22:575, 2004), fibronectin binding molecules (adnectins or monobodies; Richards et al., J. Mol. Biol. 326:1475, 2003; Parker et al., Protein Eng. Des. Selec. 18:435, 2005 and Hackel et al. (2008) J. Mol. Biol. 381:1238-1252), cysteine-knot miniproteins (Vita et al., 1995, Proc. Nat'l. Acad. Sci. (USA) 92:6404-6408; Martin et al., 2002, Nat. Biotechnol. 21:71, 2002 and Huang et al. (2005) Structure 13:755, 2005), tetratricopeptide repeat molecules (Main et al., Structure 11:497, 2003 and Cortajarena et al., ACS Chem. Biol. 3:161, 2008), leucine-rich repeat molecules (Stumpp et al., J. Mol. Biol. 332:471, 2003), lipocalin molecules (see, e.g., WO 2006/095164, Beste et al., Proc. Nat'l. Acad. Sci. (USA) 96:1898, 1999 and Schönfeld et al., Proc. Nat'l. Acad. Sci. (USA) 106:8198, 2009), V-like molecules (see, e.g., US 2007/0065431), C-type lectin molecules (Zelensky and Gready, FEBS J. 272:6179, 2005; Beavil et al., Proc. Nat'l. Acad. Sci. (USA) 89:753, 1992 and Sato et al., Proc. Nat'l. Acad. Sci. (USA) 100:7779, 2003), mAb2 or Fc-region with antigen binding molecule (Fcab™ (F-Star Biotechnology, Cambridge UK; see, e.g., WO 2007/098934 and WO 2006/072620), armadillo repeat proteins (see, e.g., Madhurantakam et al., Protein Sci. 21: 1015, 2012; WO 2009/040338), affilin (Ebersbach et al., J. Mol. Biol. 372: 172, 2007), affibody, avimers, knottins, fynomers, atrimers, cytotoxic T-lymphocyte associated protein-4 (Weidle et al., Cancer Gen. Proteo. 10:155, 2013), or the like (Nord et al., Protein Eng. 8:601, 1995; Nord et al., Nat. Biotechnol. 15:772, 1997; Nord et al., Euro. J. Biochem. 268:4269, 2001; Binz et al., Nat. Biotechnol. 23:1257, 2005; Boersma and Plückthun, Curr. Opin. Biotechnol. 22:849, 2011).

Peptide aptamers include a peptide loop (which is specific for a target peptide) attached at both ends to a protein scaffold. This double structural constraint increases the binding affinity of peptide aptamers to levels comparable to antibodies. The variable loop length is typically 8 to 20 amino acids and the scaffold can be any protein that is stable, soluble, small, and non-toxic. Peptide aptamer selection can be made using different systems, such as the yeast two-hybrid system (e.g., Gal4 yeast-two-hybrid system), or the LexA interaction trap system.

(iv) Screening and Isolation of Phage/Candidate Binding Molecule Complexes. A phage-display library as described herein can be exposed to a candidate binding molecule to assess which DMS peptides in the library are bound by the candidate binding molecule. As indicated previously, one benefit of the currently disclosed methods is that this exposure can be accomplished using incubation within a single tube or well. In particular embodiments, this phage-display library screening step is carried out by inducing the phage to display the expressed peptides on the surface of the phage clones and incubating the DMS peptide-expressing phage with a candidate binding molecule.

Herein, a clone is a phage expressing a DMS protein or peptide based on introduction of a genetic sequence encoding the DMS protein or peptide into the phage.

In particular embodiments, incubation can occur in a blocked cell culture receptacle, such as a cell culture flask, tube, and/or cell culture plate. Cell culture plates can include a single well, 6-wells, 12-wells, 24-wells, 48-wells, 96-wells, etc. In particular embodiments, the cell culture receptacle is blocked with 3% bovine serum albumin (BSA) and Tris-buffered saline-Tween (TBST). Amplified phage and candidate binding molecules can be added to the blocked cell culture receptacle. In particular embodiments, 1 mL of amplified phage at 2×10⁵-fold representation is added. In particular embodiments, 1 ng, 2 ng, 3 ng, 4 ng, 5 ng, 6 ng, 7 ng, 8 ng, 9 ng, or 10 ng of candidate binding molecule is added to the cell culture receptacle. In particular embodiments, the amount of input candidate binding molecule should not exceed the binding capacity of the beads or competitive binding used during immunoprecipitation to avoid reduction in enrichment efficiencies. Phage/candidate binding molecule complexes can be formed by rotating the plate, for example, at 4° C. for 20 hours, at 4° C. for 18 hours, or at 37° C. for 1 hour or any other appropriate temperature and time combination.

Following exposure of the phage-display library to candidate binding molecules under conditions that allow complex formation, bound complexes can be isolated from unbound phage. Any suitable method that detects interactions between molecules can be used to identify bound complexes including, e.g., immunoprecipitation, co-immunoprecipitation, ELISA, bimolecular fluorescence complementation, affinity electrophoresis, pull-down assays, label transfer, and the like. In particular embodiments isolation binding molecules (IBM) are used to separate the phage to only include phage expressing peptides that specifically bound the candidate binding molecule through immunoprecipitation. Immunoprecipitation can be conducted in the solid or mobile phase.

As indicated, in immunoprecipitation, IBM can be immobilized on a solid substrate. Useful solid substrates include materials with (i) chemical groups that can be modified for covalent attachment of IBM, (ii) low nonspecific binding characteristics, and (iii) mechanical and chemical stability. Exemplary solid substrates include beads, magnetic beads, microtiter wells, assay plates, slides, agarose, superflow agarose, UltraLink Biosupport, and the like. Most often IBM are attached to the solid support with covalent chemical interactions, but indirect coupling approaches may also be used. In particular embodiments, the solid support includes magnetic beads, such as Dynabeads (Thermo Fisher Scientific, Waltham, Mass.), Protein A beads, and/or Protein G beads.

In particular embodiments, immunoprecipitation can be performed in a blocked cell culture receptacle, such as those described above. To immunoprecipitate phage/candidate binding molecule complexes, beads associated with an IBM are added to each well and incubated. For example, in particular embodiments 40 μL of a 1:1 mix of protein A and protein G Dynabeads (Invitrogen) can be added to each well. In particular embodiments, the incubation includes rotation at 4° C. for 4 hours. As indicated above, however, the amount of beads, ratio, temperature and duration of the immunoprecipitation incubation can be adjusted as appropriate. The conditions result in binding of appropriate IBM-beads to peptide/candidate binding molecule complexes.

After this immunoprecipitation incubation, a separator can be used to isolate bound or unbound beads and isolated beads can be washed with wash buffer. In particular embodiments, the separator includes a magnet (e.g., magnetic plate) and/or centrifuge. In particular embodiments, the magnet includes a Magnetic Particle Concentrator (Thermo Fisher Scientific, Waltham, Mass.). In particular embodiments the beads are washed 1 to 5 (e.g., 3) times. In particular embodiments, the beads are washed with an appropriate amount (e.g., 400 μL) of wash buffer, such as a wash buffer including 50 mM Tris-HCl, 150 mM NaCl, and 0.1% NP-40 at a pH of 7.5.

When tag sequences are included as part of a DMS peptide, cognate binding molecules for the tag sequence can be used to isolate bound complexes. Conjugate binding molecules that specifically bind tag cassette sequences disclosed herein are commercially available. For example, His tag antibodies are commercially available from suppliers including Life Technologies, Pierce Antibodies, and GenScript. Flag tag antibodies are commercially available from suppliers including Pierce Antibodies, GenScript, and Sigma-Aldrich. Xpress tag antibodies are commercially available from suppliers including Pierce Antibodies, Life Technologies and GenScript. Avi tag antibodies are commercially available from suppliers including Pierce Antibodies, IsBio, and Genecopoeia. Calmodulin tag antibodies are commercially available from suppliers including Santa Cruz Biotechnology, Abcam, and Pierce Antibodies. HA tag antibodies are commercially available from suppliers including Pierce Antibodies, Cell Signal and Abcam. Myc tag antibodies are commercially available from suppliers including Santa Cruz Biotechnology, Abcam, and Cell Signal. Strep tag antibodies are commercially available from suppliers including Abcam, Iba, and Qiagen.

In particular embodiments, bound complexes are separated using affinity chromatography. Affinity chromatography refers generally to chromatographic procedures that rely on the specific affinity between a substance to be isolated and a molecule that it can specifically bind to. In particular embodiments, affinity chromatography can be accomplished using columns or beads or other surfaces coated in antibodies or other relevant binding domains. Column material is synthesized by covalently coupling one of the binding partners to an insoluble matrix. The column material is then able to specifically adsorb the substance from the solution. Affinity chromatography includes microfluidic affinity chromatography.

Elution is not necessary in all methods of isolation. Elution is the process of extracting one material from another through a washing step. Elution occurs by changing the conditions to those in which binding will not occur (alter pH, ionic strength, temperature, etc.). Common elution buffers utilizing changes in pH include glycine HCl, citric acid, triethylamine, triethanolamine, or ammonium hydroxide. Common elution buffers utilizing changes in ionic strength and/or chaotropic effects include magnesium chloride in Tris, lithium chloride in phosphate buffer, sodium iodide, or sodium thiocyanate. Common elution buffers utilizing denaturing include guanidine-HCl, urea, deoxycholate, or SDS. Other elution options include competitive binding or organic elution buffers. In particular embodiments, the isolated phage is not eluted.

In particular embodiments, eluted phage can be propagated and/or subjected to further rounds of screening (e.g., a subsequent round of incubating and potential capture following exposure to a candidate binding molecules). Such subsequent rounds can increase the number of enriched phage specifically to the candidate binding molecule.

(v) Nucleotide Processing, Sequencing, and Analysis. Once selected phage are isolated and/or purified, for example as described above, the bound peptides can be is identified. In particular embodiments, the bound peptides are identified by obtaining nucleotides from the selected phages and sequencing the nucleotides to obtain the genetic information encoding the bound peptides. As described elsewhere herein, particular embodiments can include sequencing nucleotides from bound and/or non-bound phage. Numerous methods can be used to amplify and/or sequence phage nucleotides within the systems and methods disclosed herein.

In particular embodiments, phage selected for sequencing can be lysed and the phage nucleotides can be used as a template for amplification and sequencing. The term “nucleotides” includes the terms “oligonucleotide” and “polynucleotide” and refers to single-stranded or double-stranded polymers of nucleotide monomers, including 2′-deoxyribonucleotides (DNA) and ribonucleotides (RNA). The nucleic acid can be composed deoxyribonucleotides or ribonucleotides linked by internucleotide phosphodiester bond linkages, and associated counter-ions, e.g., H+, NH4+, trialkylammonium, Mg2+, Na+ and the like. Nucleotides and nucleic acides are used interchangeably herein.

In particular embodiments, phage are lysed by incubating at 95° C. for 10 mins.

The nucleotides of the phage can be extracted and purified using any suitable technique. A number of techniques are known in the art, and kits to practice the techniques are commercially available. Commercially available DNA extraction kit include genomic DNA Extraction Kits from Thermo Fisher Scientific, BioVision (e.g., catalog #K281 and K309) and Bio-Rad, to name a few.

RNA particularly can be extracted using TRIzol (Invitrogen, Carlsbad, Calif.) and purified using RNeasy FFPE Kit (Qiagen, Valencia, Calif.). RNA can be further purified using DNAse I treatment (Ambion, Austin, Tex.) to eliminate any contaminating DNA. RNA concentrations can be made using a Nanodrop ND-1000 spectrophotometer (Nanodrop Technologies, Rockland, Del.). RNA can be further purified to eliminate contaminants that interfere with cDNA synthesis by cold sodium acetate precipitation. RNA integrity can be evaluated by running electropherograms, and RNA integrity number (RIN, a correlative measure that indicates intactness of mRNA) can be determined using the RNA 6000 PicoAssay for the Bioanalyzer 2100 (Agilent Technologies, Santa Clara, Calif.).

In particular embodiments in which nucleic acids are extracted for sequencing, the nucleic acids can be subjected to one or more preparative reactions. These preparative reactions can include, for example, in vitro transcription (IVT), labeling, fragmentation, amplification and/or other reactions.

Nucleic acids to be sequenced can be fragmented to a desired size range by fragmentation methods that include enzymatic, chemical, mechanical, or in vitro transposition means. Such fragmentation methods are known in the art and utilize standard molecular methods such as nebulization or sonication. In particular embodiments, a nucleic acid fragment includes a portion or all of a nucleic acid molecule. In particular embodiments, the amount of nucleic acid to be fragmented include 1 to 500 ng, 1 to 250 ng, 1 to 100 ng, 10 to 100 ng, and 5 to 50 ng.

Fragmentation of nucleic acid molecules can result in nucleic acid fragments with a heterogeneous mix of blunt and 3′- and 5-overhanging ends. After fragmentation of nucleic acid molecules to a desired size range, the fragmented nucleic acids molecules can be modified for ease of ligation to adapters. In particular embodiments, A-tails or T-tails can be added to the nucleic acid fragments to facilitate ligation to adapters. A-tailing is the addition of non-templated adenosine overhangs to the 3′ end of a double-stranded nucleic acid molecule. A-tailed nucleic acids can be useful for ligation to adapters with a T-overhang at the 3′ end. T-tails are non-templated thymine overhangs added to the 3′ end of a double-stranded nucleic acid molecule. T-tails can be useful for ligation to A-tailed adapters. Enzymes that can add 3′ A-tails or T-tails to double stranded nucleic acids include Taq polymerase, terminal transferase, poly(A) polymerase, Klenow and Klenow fragment.

Adapters can include any nucleic acid sequences suitable for sequencing. For example, adapters can be compatible with: sequencing by synthesis (such as P7 and P5 adapters (Illumina, San Diego, Calif.)); pyrosequencing (Roche Applied Science, Basel, Switzerland)); rolling circle amplification sequencing (adapters available from BGI Genomics, Shenzhen, Guangdong, China); sequencing by ligation (adapters available for SOLiD systems from Thermo Fisher, Waltham, Mass.); and Sanger sequencing by synthesis. In particular embodiments, adapters are composed of nucleotide sequences that: allow immobilization of a nucleic acid fragment to a solid surface for sequencing; provide primer binding sites for amplification of the nucleic acid fragment; add additional functional sequences to the adapter during amplification; and/or provide regions on the nucleic acid fragment from which the sequencing process can start. Adapters can be partially single-stranded, due to the presence of one or more regions of non-complementarity between the sense strand and the antisense strand, and partially double-stranded or capable of forming a duplex structure, due to the presence of one or more regions of complementarity between the sense and antisense strands.

Adapters are described in, for example, US20070172839, WO2009133466, CN102061335B, U.S. Pat. Nos. 8,420,319, 8,883,990, and Ahn et al. (2017) Scientific Reports 7:46678.

Exemplary adapter sequences can include an Illumina TruSeq universal adapter sequence 5′-AATGATACGGCGACCACCGAGATCTACACTCTTTCCCTACACGACGCTCTTCC GATCT-3′ (SEQ ID NO: 87) and an Illumina TruSeq Index adapter sequence 5′-GATCGGAAGAGCACACGTCTGAACTCCAGTCACNNNNNNATCTCGTATGCCGTCTTCTGCT TG-3′ (SEQ ID NO: 88), where “N” is any nucleotide, and the 6 Ns together are a unique sequence which can readily be identified as unique to a given sequencing library (Illumina, San Diego, Calif.).

The Applied Biosystems SOLiD™ System sequencing platform for DNA uses truncated-TA adapters for capture of the DNA on the microarray and pre-capture amplification by PCR. See Protocol Version 2.1, Baylor College of Medicine, Human Genome Sequencing Center, “Preparation of SOLiD™ System Fragment Libraries for Targeted Resequencing using NimbleGen Microarrays or Solution Phase Sequence Capture.” In a further example, the Applied Biosystems SOLiD 4 System employs P1 and P2 adapters for sequencing and PCR primer recognition as set forth in the Library Preparation Guide (April 2010). Adapters which provide priming sequences for both amplification and sequencing of library fragments for use with the 454 Life Science GS20 sequencing system are described by F. Cheung, et al. BMC Genomics 2006, 7:272. Other adapters are described elsewhere herein and can also be used (see, e.g., the Experimental Examples).

“Amplification” refers to any process of producing at least one copy of a nucleic acid and in many cases produces multiple copies. An amplification product can be RNA or DNA and may include a complementary strand to an expressed target sequence. DNA amplification products can be produced initially through reverse translation and then optionally from further amplification reactions. The amplification product may include all or a portion of a target sequence and may optionally be labeled. A variety of amplification methods are suitable for use, including polymerase-based methods and ligation-based methods.

Exemplary PCR types include allele-specific PCR, assembly PCR, asymmetric PCR, endpoint PCR, hot-start PCR, in situ PCR, intersequence-specific PCR, inverse PCR, linear after exponential PCR, ligation-mediated PCR, methylation-specific PCR, miniprimer PCR, multiplex ligation-dependent probe amplification, multiplex PCR, nested PCR, overlap-extension PCR, polymerase cycling assembly, qualitative PCR, quantitative PCR, real-time PCR, single-cell PCR, solid-phase PCR, thermal asymmetric interlaced PCR, touchdown PCR, universal fast walking PCR, etc.

Techniques to accelerate PCR can be used, for example centrifugal PCR, which allows for greater convection within the sample, and includes infrared heating steps for rapid heating and cooling of the sample. One or more cycles of amplification can be performed. An excess of one primer can be used to produce an excess of one primer extension product during PCR; preferably, the primer extension product produced in excess is the amplification product to be detected. A plurality of different primers may be used to amplify different target nucleic acids or different regions of particular target nucleic acids within the sample.

PCR and LCR are driven by thermal cycling. Alternative amplification reactions, which may be performed isothermally, can also be used. Exemplary isothermal techniques include branched-probe DNA assays, cascade-RCA, helicase-dependent amplification, loop-mediated isothermal amplification (LAMP), nucleic acid-based amplification (NASBA), nicking enzyme amplification reaction (NEAR), PAN-AC, Q-beta replicase amplification, rolling circle replication (RCA), self-sustaining sequence replication (3SR), strand-displacement amplification, and ribozyme-based methods.

The first cycle of amplification in polymerase-based methods typically forms a primer extension product complementary to the template strand. If the template is single-stranded RNA, a polymerase with reverse transcriptase activity is used in the first amplification to reverse transcribe the RNA to DNA, and additional amplification cycles can be performed to copy the primer extension products. The primers for a PCR must, of course, be designed to hybridize to regions in their corresponding template that can produce an amplifiable segment; thus, in particular embodiments, each primer must hybridize so that its 3′ nucleotide is paired to a nucleotide in its complementary template strand that is located 3′ from the 3′ nucleotide of the primer used to replicate that complementary template strand in the PCR. As is well understood by one of ordinary skill in the art, the terms “hybridization” and “hybridize” refer to the formation of complexes between nucleotide sequences which are sufficiently complementary to form complexes via Watson-Crick base pairing. Hybridization technologies that may be used with assays and detections methods described herein are described in, for example, U.S. Pat. Nos. 5,143,854; 5,288,644; 5,324,633; 5,432,049; 5,470,710; 5,492,806; 5,503,980; 5,510,270; 5,525,464; 5,547,839; 5,580,732; 5,661,028; and 5,800,992 as well as WO 95/21265; WO 96/31622; WO 97/10365; WO 97/27317; EP 373 203; and EP 785 280.

The target nucleic acids can be amplified by contacting one or more strands of the target nucleic acids with a primer and a polymerase having suitable activity to extend the primer and copy the target nucleic acids to produce full-length complementary nucleic acids or smaller portions thereof. Any enzyme having a polymerase activity that can copy the target nucleic acids can be used, including DNA polymerases, RNA polymerases, reverse transcriptases, and/or enzymes having more than one type of polymerase or enzyme activity. The enzyme can be thermolabile or thermostable. Mixtures of enzymes can also be used. Exemplary enzymes include: DNA polymerases such as DNA Polymerase I (Pol 1), the Klenow fragment of Pol I, T4, T7, Sequenase® (GE Healthcare, Limited, UK) T7, Sequenase® Version 2.0 T7, Tub, Taq, Tth, Pfic, Pfu, Tsp, Tfl, Tli and Pyrococcus sp GB-D DNA polymerases; RNA polymerases such as E. coli, SP6, T3 and T7 RNA polymerases; and reverse transcriptases such as AMV, M-MuLV, MMLV, RNAse H MMLV (SuperScript® (Life Technologies Corporation, Carlsbad, Calif.), SuperScript® II, ThermoScript (Invitrogen, Carlsbad, Calif.), and HIV-1 and RAV2 reverse transcriptases. All of these enzymes are commercially available.

Suitable reaction conditions are chosen to permit amplification of the target nucleic acids, including pH, buffer, ionic strength, presence and concentration of one or more salts, presence and concentration of reactants and cofactors such as nucleotides and magnesium and/or other metal ions (e.g., manganese), optional cosolvents, temperature, thermal cycling profile for amplification schemes including PCR, and may depend in part on the polymerase being used as well as the nature of the sample. Cosolvents include formamide (typically at from 2 to 10%), glycerol (typically at from 5 to 10%), and DMSO (typically at from 0.9 to 10%). Techniques may be used in the amplification scheme in order to minimize the production of false positives or artifacts produced during amplification. These include “touchdown” PCR, hot-start techniques, use of nested primers, or designing PCR primers so that they form stem-loop structures in the event of primer-dimer formation and thus are not amplified. See, e.g., Fakruddin et al., J Pharm Bioallied Sci. 5:245 (2013) for a review of amplification methods.

In particular embodiments, a reference sample can be assayed to ensure reagent and process stability. Negative controls (e.g., no template) can be assayed to monitor any exogenous nucleic acid contamination.

In particular embodiments, the methods include quantifying and/or detecting an endogenous control. An endogenous control can refer to a sequence that has a known copy number in a phage library. In particular embodiments, measuring the endogenous control sequence can be useful for determining the copy number of DMS peptide sequences. In particular embodiments, methods that include quantifying an endogenous control can be useful for determining the percentage of phage within a sample. An exemplary method of quantifying the copy number of a gene (e.g., a unique DMS peptide encoding sequence) using an endogenous control can be found in Ma & Chung, Curr Protoc Hum Genet. 80: 7.21.1-7.21.8, 2014.

In particular embodiments, the methods include detecting an exogenous control. Exogenous control can refer to a nucleotide sequence that is “spiked” into a sample. In particular embodiments, the exogenous control is spiked into the sample at a known quantity (e.g., known copy number), which can be useful, for example, to determine the absolute quantity of a gene sequence (e.g., a unique DMS peptide encoding sequence).

In particular embodiments, the amplification can be performed by sample partition dPCR (spdPCR). An example of sample partition ddPCR is Droplet Digital PCR.

Droplet digital PCR (ddPCR) allows accurate quantification of phage sequences (e.g., Droplet Digital™ PCR (ddPCR™) (Bio-Rad Laboratories, Hercules, Calif.)). ddPCR™ technology uses a combination of microfluidics and surfactant chemistry to divide PCR samples into water-in-oil droplets. Hindson et al., Anal. Chem. 83(22): 8604-8610, 2011. The droplets support PCR amplification of the target template molecules they contain and use reagents and workflows similar to those used for most standard Taqman probe-based assays.

Following PCR, each droplet is analyzed or read in a flow cytometer to determine the fraction of PCR-positive droplets in the original sample. These data are then analyzed using Poisson statistics to determine the target concentration in the original sample. See Bio-Rad Droplet Digital™ (ddPCR™) PCR Technology.

While ddPCR™ is a preferred spdPCR approach, other sample partition PCR methods based on the same underlying principles may also be used to divide samples into discrete partitions (e.g., droplets). Exemplary partitioning methods and systems include use of one or more of emulsification, droplet actuation, microfluidics platforms, continuous-flow microfluidics, reagent immobilization, and combinations thereof. In particular embodiments, partitioning is performed to divide a sample into a sufficient number of partitions such that each partition contains one or zero nucleic acid molecules. In particular embodiments, the number and size of partitions is based on the concentration and volume of the bulk sample.

Methods and devices for partitioning a bulk volume into partitions by emulsification are described in Nakano et al (J Biotechnol 102:117-124, 2003) and Margulies et al. (Nature 437:376-380, 2005). Systems and methods to generate “water-in-oil” droplets are described in U.S. Publication No. 2010/0173394. Microfluidics systems and methods to divide a bulk volume into partitions are described in U.S. Publication Nos. 2010/0236929; 2010/0311599; and 2010/0163412, and U.S. Pat. No. 7,851,184. Microfluidic systems and methods that generate monodisperse droplets are described in Kiss et al. (Anal Chem. 80(23):8975-8981, 2008). Further microfluidics systems and methods for manipulating and/or partitioning samples using channels, valves, pumps, etc. are described in U.S. Pat. No. 7,842,248. Continuous-flow microfluidics systems and methods are described in Kopp et al. (Science, 280:1046-1048, 1998).

Partitioning methods can be augmented with droplet manipulation techniques, including electrical (e.g., electrostatic actuation, dielectrophoresis), magnetic, thermal (e.g., thermal Marangoni effects, thermocapillary), mechanical (e.g., surface acoustic waves, micropumping, peristaltic), optical (e.g., opto-electrowetting, optical tweezers), and chemical means (e.g., chemical gradients). In particular embodiments, a droplet microactuator is supplemented with a microfluidics platform (e.g. continuous flow components).

Particular embodiments use a droplet microactuator. A droplet microactuator can be capable of effecting droplet manipulation and/or operations, such as dispensing, splitting, transporting, merging, mixing, agitating, and the like. Droplet operation structures and manipulation techniques are described in U.S. Publication Nos. 2006/0194331 and 2006/0254933 and U.S. Pat. Nos. 6,911,132; 6,773,566; and 6,565,727.

In particular embodiments, nucleic acid targets, primers, and/or probes are immobilized to a surface, for example, a substrate, plate, array, bead, particle, etc. Immobilization of one or more reagents provides (or assists in) one or more of: partitioning of reagents (e.g. target nucleic acids, primers, probes, etc.), controlling the number of reagents per partition, and/or controlling the ratio of one reagent to another in each partition. In particular embodiments, assay reagents and/or target nucleic acids are immobilized to a surface while retaining the capability to interact and/or react with other reagents (e.g. reagent dispensed from a microfluidic platform, a droplet microactuator, etc.). In particular embodiments, reagents are immobilized on a substrate and droplets or partitioned reagents are brought into contact with the immobilized reagents. Techniques for immobilization of nucleic acids and other reagents to surfaces are well understood by those of ordinary in the art. See, for example, U.S. Pat. No. 5,472,881 and Taira et al. (Biotechnol. Bioeng. 89(7):835-8, 2005).

Amplification reagents can be added to a sample prior to partitioning, concurrently with partitioning and/or after partitioning has occurred. In particular embodiments, all partitions are subjected to amplification conditions (e.g. reagents and thermal cycling), but amplification only occurs in partitions containing target nucleic acids (e.g. nucleic acids containing sequences complementary to primers added to the sample). The template nucleic acid can be the limiting reagent in a partitioned amplification reaction. In particular embodiments, a partition contains one or zero target (e.g. template) nucleic acid molecules.

Detection methods can be utilized to identify sample partitions containing amplified target(s). Detection can be based on one or more characteristics of a sample partition such as a physical, chemical, luminescent, or electrical aspects, which correlate with amplification.

In particular embodiments, following amplification, sample partitions containing amplified target(s) are sorted from sample partitions not containing amplified targets or from sample partitions containing other amplified target(s). In particular embodiments, individual sample partitions are isolated for subsequent manipulation, processing, and/or analysis of the amplified target(s) therein. In particular embodiments, sample partitions containing similar characteristics (e.g. same fluorescent labels, similar nucleic acid concentrations, etc.) are grouped (e.g. into packets) for subsequent manipulation, processing, and/or analysis.

Particular embodiments can utilize next generation sequencing (NGS) to sequence nucleotides, such as amplified nucleotide products. In particular embodiments, a single NGS run can be performed on nucleic acid molecules from selected phage that have been selected from one or more phage library screens. In particular embodiments, DNA sequencing with commercially available NGS platforms may be conducted with the following steps. As indicated, first, DNA sequencing libraries may be generated by clonal amplification by PCR in vitro. Second, the DNA may be sequenced by synthesis, such that the DNA sequence is determined by the addition of nucleotides to the complementary strand rather through chain-termination chemistry. Third, the spatially segregated, amplified DNA templates may be sequenced simultaneously in a massively parallel fashion without the requirement for a physical separation step. While these steps are followed in most NGS platforms, each utilizes a different strategy (see e.g., Anderson & Schrijver, Genes, 1: 38-69, 2010).

Particular embodiments of NGS systems include any sequencing system that automates steps in the sequencing process and/or includes components that allow for high-throughput sequencing. In particular embodiments, NGS includes automated Sanger sequencing, sequencing by synthesis, pyrosequencing, sequencing by ligation, rolling amplification sequencing, single molecule sequencing, and nanopore sequencing. In particular embodiments, the sequence reads can allow generation of consensus sequences.

Sequencing-by-synthesis (SBS) is an exemplary sequencing method. SBS can be carried out as follows: To initiate a first SBS cycle, one or more labeled nucleotides, DNA polymerase, SBS primers etc., can be contacted with a nucleic acid to be sequenced. A labeled nucleotide incorporated during SBS primer extension can be detected. Optionally, the nucleotides can include a reversible termination moiety that terminates further primer extension once a nucleotide has been added to the SBS primer. For example, a nucleotide analog having a reversible terminator moiety can be added to a primer such that subsequent extension cannot occur until a deblocking agent is encountered to remove the moiety. Thus, for embodiments that use reversible termination, a deblocking reagent can be delivered during sequencing (before or after detection occurs). Washes can be carried out between the various delivery steps. The cycle can then be repeated n times to extend the primer by n nucleotides, thereby detecting a sequence of length n. In particular embodiments, sequencing by synthesis includes Sanger sequencing. Exemplary SBS procedures, fluidic systems and detection platforms that can be adapted for use with systems and methods of the present disclosure are described, for example, in Bentley et al., Nature 456:53-59, 2008; WO1991/006678; WO2004/018497; WO2007/123744; U.S. Pat. Nos. 7,057,026; 7,329,492; 7,211,414; 7,315,019; 7,405,281; and US20080108082.

Other sequencing procedures that use cyclic reactions can be used, such as pyrosequencing. Pyrosequencing detects the release of inorganic pyrophosphate (PPi) as particular nucleotides are incorporated into a nascent nucleic acid strand (Ronaghi et al., Analytical Biochemistry 242(1): 84-89, 1996; Ronaghi, Genome Res. 11 (1), 3-11, 2001; Ronaghi et al., Science 281(5375): 363, 1998; U.S. Pat. Nos. 6,210,891; 6,258,568; and 6,274,320). In pyrosequencing, released PPi can be detected by being immediately converted to adenosine triphosphate (ATP) by ATP sulfurylase, and the level of ATP generated can be detected via luciferase-produced photons. Thus, the sequencing reaction can be monitored via a luminescence detection system. Excitation radiation sources used for fluorescence-based detection systems are not necessary for pyrosequencing procedures. Useful fluidic systems, detectors and procedures that can be used for application of pyrosequencing to systems and methods of the present disclosure are described, for example, in WO2012/058096; US20050191698; U.S. Pat. Nos. 7,595,883; and 7,244,559.

Sequencing by rolling circle amplification can be used in systems and methods of the present disclosure. In rolling circle amplification, circular templates are amplified to generate long concatamers called DNA nanoballs. The nanoballs can be immobilized on a flow cell for sequencing. Rolling circle amplification is described, for example, in Xu et al. BMC Bioinformatics (2019) 20:153; Korfhage et al Biology Methods and Protocols 2(1), January 2017, bpx007; Wu et al Biotechniques 34(1): 204-207, 2003: Predki et al Methods Mol Biol. 255: 189-196, 2004 U.S. Pat. Nos. 6,221,603; 6,783,943; 9,624,538; US 2005/0069939; and WO 2015/079042.

Sequencing-by-ligation can be used in systems and methods of the present disclosure. Sequencing-by-ligation includes the hybridization and ligation of labeled probe and anchor sequences to a nucleic acid strand. The probes encode one or two known bases and a series of degenerate bases to drive complementary binding between the probe and template nucleic acid strand to be sequenced. The anchor sequence includes a known sequence that is complementary to an adapter sequence and provides a site to initiate ligation. After ligation, the template can be imaged and the known bases in the probe identified. This sequencing process can be repeated after removal of the anchor-probe complex or cleavage of the fluorophore from the probe and regeneration of a site to initiate ligation. Sequencing-by-ligation is described in, for example, Shendure et al., Science 309:1728-1732, 2005; U.S. Pat. Nos. 5,599,675; and 5,750,341.

Particular sequencing embodiments can utilize methods involving real-time monitoring of DNA polymerase activity. For example, nucleotide incorporations can be detected through fluorescence resonance energy transfer (FRET) interactions between a fluorophore-bearing polymerase and γ-phosphate-labeled nucleotides, or with zeromode waveguides (ZMWs). ZMWs include a specialized flow cell with many thousands of individual picoliter wells with transparent bottoms. Techniques and reagents for FRET-based sequencing are described, for example, in Levene et al., Science 299: 682-686, 2003; Lundquist et al., Opt. Lett. 33: 1026-1028, 2008; and Korlach et al., Proc. Natl. Acad. Sci. USA 105:1176-1181, 2008. In particular embodiments, single molecule real time sequencing platforms such as the SMRT platform from Pacific Biosciences (Menlo Park, Calif.) uses ZMWs and a polymerase affixed to the bottom of each well.

Particular sequencing embodiments include detection of a proton released upon incorporation of a nucleotide into an extension product. For example, sequencing based on detection of released protons can use an electrical detector and associated techniques that are commercially available from Ion Torrent (Guilford, Conn.; a Life Technologies and Thermo Fisher subsidiary) or sequencing methods and systems described for instance in US20090026082; US20090127589; US20100137143; and US20100282617.

Particular sequencing embodiments include detection of a nucleic acid sequence based on current modulation as a nucleic acid molecule passes through a nanopore that has a current passing through it. In particular embodiments, the nucleic acid molecule is translocated through the nanopore via the action of a secondary motor protein. In particular embodiments, nanopore sequencing is provided by a platform such as the MinION from Oxford Nanopore Technologies (Oxford, United Kingdom). Nanopore sequencing is described in, for example, Clarke et al. Nat. Nanotechnol. 4: 265-270 (2009); U.S. Pat. Nos. 9,279,153; 9,322,820; 9,377,432; 9,546,400; WO 2015/081294: and WO 2017/083828.

Examples of commercially available NGS platforms include:

| | Template | | Read Length |
|---|---|---|---|
| Platform | Preparation | Chemistry | (bases) |
| Roche 454 | Clonal-emPCR | Pyrosequencing | 400 |
| GS FLX Titanium | Clonal-emPCR | Pyrosequencing | 400 |
| Illumina | Clonal Bridge | Reversible Dye | 35-100 |
| | Amplification | Terminator | |
| HiSeq 2000 | Clonal Bridge | Reversible Dye | 35-100 |
| | Amplification | Terminator | |
| Genom Analyzer | Clonal Bridge | Reversible Dye | 35-100 |
| IIX, IIE | Amplification | Terminator | |
| IScanSQ | Clonal Bridge | Reversible Dye | 35-75 |
| | Amplification | Terminator | |
| Life Technologies | Clonal-emPCR | Oligonucleotide | 35-50 |
| Solid 4 | | Probe Ligation | |
| Helicos Biosciences | Single Molecule | Reversible Dye | 35 |
| Heliscope | | Terminator | |
| Pacific Biosciences | Single Molecule | Phospholinked | 800-1000 |
| SMART | | Fluorescent | |
| | | Nucleotides | |

Particular sequencing embodiments include de novo peptide sequencing which sequences and identifies a peptide from observed tandem mass spectrometry (MS/MS) spectrum. In a tandem mass spectrometer, many copies of the peptide backbone can be broken up into fragments. The fragment ions are measured to produce MS/MS spectrum which is a plot of peaks of the mass to charge values of the corresponding fragments. Based on the mass and/or charge differences between fragments, each residue of the peptide can be identified.

In particular embodiments, the fragmentation method includes Collision-Induced Dissociation (CID). In particular embodiments, the fragmentation method includes Electron-Transfer Dissociation (ETD).

Following sequencing, computational methods can be applied to facilitate protein residue mapping. For example, enriched sequences can be aligned and regions of overlap can identify residues important for binding between the protein of interest and the candidate binding molecule. Enriched DMS peptides that span epitope regions have mutations that have no effect of binding between the protein of interest and the epitope, and DMS peptides spanning the epitope region that are not enriched have mutations that result in loss of binding of the protein of interest to its epitope. For example, in particular embodiments, a bioinformatics analysis method can include plotting the fold enrichment of wildtype peptides. The region of the epitope is determined by observing which peptides are highly enriched above background. Then, within that region, the effect of each mutation to the wildtype amino acids is closely examined. The scaled differential selection is calculated and plotted. The plot is a visual representation which mutations result in a loss of binding (or result in improved binding). A more detailed example of calculating differential selection can be found in Bloom, Biology Direct, 12:1, 2017.

Particular embodiments include plotting the enrichment of wildtype peptides and determining the region of the epitope by determining which peptides are highly enriched above background. Within that region, the effect of each mutation to the wildtype sequence can be computed and plotted to visually represent mutations that result in a loss of binding or improved binding. Aspects of this process may be practiced using differential selection calculations, also as described in Bloom, Biology Direct, 12:1, 2017.

In particular embodiments, a bioinformatics analysis method can include determining a zero-inflated generalized Poisson significant-enrichment assignment algorithm that can be used to generate a −log 10(p-value) for enrichment of each clone across all samples. A reproducibility threshold can be established to call ‘hits’ in technical replicate pairs by first calculating the log 10(−log 10(p-value)) for each clone in Replicate 1. These values can then be surveyed in Replicate 2 by using a sliding window of width 0.01 from −2 to the maximum log 10(−log 10(p-value)) value in Replicate 1. For all clones that fall within each window, the median and median absolute deviation of log 10(−log 10(p-values)) in Replicate 2 can be calculated and plotted against the window location. The reproducibility threshold can be set as the window location where the median was greater than the median absolute deviation. The distribution of the threshold −log 10(p-values) is centered around a median of 2.2. In sum, a phage clone is called a ‘hit’ if the −log 10(p value) is at least 2.2 in both replicates. Beads-only samples, which serve as a negative control for non-specific binding of phage, can be used to identify and eliminate background hits. Peptides called as hits are then aligned using Clustal Omega. The shortest amino acid sequence present in all of the hits is defined as the “minimal binding epitope” of a candidate binding molecule (Larman, et al., Nat Biotechnol, 29(6):535-541, 2011).

In particular embodiments, a bioinformatics analysis method can include determining the position weight matrix (PWM) spanning the epitope region to determine the motifs that are enriched in the presence of the protein of interest. A matrix of the frequency of each amino acid at every position is determined by observing the number of clones with a specific amino acid enriched by the protein of interest, as compared to the background. The log₂ of the relative frequency of an amino acid can be plotted on a logo plot, and the motif displayed corresponds to the epitope of the protein of interest (Stormo, et al., Nucleic Acids Res., 10(9): 2997-3011, 1982 and Xia, Scientifica, Volume 2012, 2012).

In particular embodiments, a bioinformatics analysis method can include calculating the z-score of each peptide in order to determine clones containing amino acids that are significantly depleted or enriched. First, many replicates of the background library are sequenced deeply to obtain an expected range of frequencies of each clone and to generate a Gaussian distribution. The frequency of a clone in an experimental sample can be compared to this distribution, and a z-score is assigned based on whether it falls outside the expected standard deviation (Yuan, et al., BioRxiv, 2018, https://doi.org/10.1101/285916).

These types of analyses can be performed using computer control systems that are programmed to implement methods of the disclosure. The computer system can be an electronic device of a user or a computer system that is remotely located with respect to the electronic device. The electronic device can be a mobile electronic device.

The computer system includes a central processing unit (CPU, also “processor” and “computer processor” herein), which can be a single core or multi core processor, or a plurality of processors for parallel processing. The computer system also includes memory or memory location (e.g., random-access memory, read-only memory, flash memory), electronic storage unit (e.g., hard disk), communication interface (e.g., network adapter) for communicating with one or more other systems, and peripheral devices, such as cache, other memory, data storage and/or electronic display adapters. The memory, storage unit, interface and peripheral devices are in communication with the CPU through a communication bus, such as a motherboard. The storage unit can be a data storage unit (or data repository) for storing data. The computer system can be operatively coupled to a computer network (“network”) with the aid of the communication interface. The network can be the Internet, an internet and/or extranet, or an intranet and/or extranet that is in communication with the Internet. The network in some cases is a telecommunication and/or data network. The network can include one or more computer servers, which can enable distributed computing, such as cloud computing. The network in some cases with the aid of the computer system can implement a peer-to-peer network, which may enable devices coupled to the computer system to behave as a client or a server.

The CPU can execute a sequence of machine-readable instructions, which can be embodied in a program or software. The instructions may be stored in a memory location, such as the memory. The instructions can be directed to the CPU which can subsequently program or otherwise configure the CPU to implement methods of the present disclosure. Examples of operations performed by the CPU can include fetch, decode, execute, and writeback.

The CPU can be part of a circuit, such as an integrated circuit. One or more other components of the system can be included in the circuit. In some cases, the circuit is an application specific integrated circuit (ASIC).

The storage unit can store files, such as drivers, libraries and saved programs. The storage unit can store user data, e.g., user preferences and user programs. The computer system in some cases can include one or more additional data storage units that are external to the computer system such as located on a remote server that is in communication with the computer system through an intranet or the Internet.

The computer system can communicate with one or more remote computer systems through the network. For instance, the computer system can communicate with a remote computer system of a user. Examples of remote computer systems include personal computers (e.g., portable PC), slate or tablet PC's (e.g., Apple® iPad, Samsung® Galaxy Tab), telephones, Smart phones (e.g., Apple® iPhone, Android-enabled device, Blackberry®), or personal digital assistants. The user can access the computer system via the network.

Methods as described herein can be implemented by way of machine (e.g., computer processor) executable code stored on an electronic storage location of the computer system, such as, for example, on the memory or electronic storage unit. The machine executable or machine-readable code can be provided in the form of software. During use, the code can be executed by the processor. In some cases, the code can be retrieved from the storage unit and stored on the memory for ready access by the processor. In some situations, the electronic storage unit can be precluded, and machine-executable instructions are stored on memory.

The code can be pre-compiled and configured for use with a machine having a processer adapted to execute the code or can be compiled during runtime. The code can be supplied in a programming language that can be selected to enable the code to execute in a pre-compiled or as-compiled fashion.

Aspects of the systems and methods provided herein, such as the computer system, can be embodied in programming. Various aspects of the technology may be thought of as “products” or “articles of manufacture” typically in the form of machine (or processor) executable code and/or associated data that is carried on or embodied in a type of machine readable medium. Machine-executable code can be stored on an electronic storage unit, such as memory (e.g., read-only memory, random-access memory, flash memory) or a hard disk. “Storage” type media can include any or all of the tangible memory of the computers, processors or the like, or associated modules thereof, such as various semiconductor memories, tape drives, disk drives and the like, which may provide non-transitory storage at any time for the software programming. All or portions of the software may at times be communicated through the Internet or various other telecommunication networks. Such communications, for example, may enable loading of the software from one computer or processor into another, for example, from a management server or host computer into the computer platform of an application server. Thus, another type of media that may bear the software elements includes optical, electrical and electromagnetic waves, such as used across physical interfaces between local devices, through wired and optical landline networks and over various air-links. The physical elements that carry such waves, such as wired or wireless links, optical links or the like, also may be considered as media bearing the software. As used herein, unless restricted to non-transitory, tangible “storage” media, terms such as computer or machine “readable medium” refer to any medium that participates in providing instructions to a processor for execution.

Hence, a machine readable medium, such as computer-executable code, may take many forms including a tangible storage medium, a carrier wave medium or physical transmission medium. Non-volatile storage media include, for example, optical or magnetic disks, such as any of the storage devices in any computer(s) or the like, such as may be used to implement the databases, etc. shown in the drawings. Volatile storage media include dynamic memory, such as main memory of such a computer platform. Tangible transmission media include coaxial cables; copper wire and fiber optics, including the wires that include a bus within a computer system. Carrier-wave transmission media may take the form of electric or electromagnetic signals, or acoustic or light waves such as those generated during radio frequency (RF) and infrared (IR) data communications. Common forms of computer-readable media therefore include for example: a floppy disk, a flexible disk, hard disk, magnetic tape, any other magnetic medium, a CD-ROM, DVD or DVD-ROM, any other optical medium, punch cards paper tape, any other physical storage medium with patterns of holes, a RAM, a ROM, a PROM and EPROM, a FLASH-EPROM, any other memory chip or cartridge, a carrier wave transporting data or instructions, cables or links transporting such a carrier wave, or any other medium from which a computer may read programming code and/or data. Many of these forms of computer readable media may be involved in carrying one or more sequences of one or more instructions to a processor for execution.

The computer system can include or be in communication with an electronic display that includes a user interface (UI) for providing, for example, results of sequence analyses following exposure of a DMS-peptide phage library to a candidate binding molecule. Examples of UI's include a graphical user interface (GUI) and web-based user interface.

Methods and systems of the present disclosure can be implemented by way of one or more algorithms.

### Exemplary Embodiments

1. A method of performing protein residue mapping including:

obtaining a phage library expressing deep mutational scanning (DMS) peptides;

incubating the phage library expressing the DMS proteins or peptides in a solution including a candidate binding molecule;

separating phage bound to the candidate binding molecule from phage not bound to the candidate binding molecule using immunoprecipitation;

lysing and sequencing nucleotides of the bound and/or unbound phage; and

determining residues responsible for the binding or non-binding of phage to the candidate binding molecule based on the sequencing

thereby performing protein residue mapping.

2. The method of embodiment 1, wherein the DMS proteins or peptides are selected from a DMS library.
3. The method of embodiments 1 or 2, wherein the DMS proteins or peptides include all peptides in the DMS library.
4. The methods of any of embodiments 1-3, wherein the DMS proteins or peptides are derived from a protein of interest selected from a viral protein, a bacterial protein, a fungal protein, or a cancer cell antigen.
5. The method of embodiment 4, wherein the viral protein includes a human immunodeficiency virus-1 (HIV-1) viral protein, an HIV-2 viral protein, a simian immunodeficiency virus (SIV) viral protein, an influenza virus viral protein, an Ebola virus viral protein, a coronavirus (CoV) viral protein, a Lassa virus viral protein, a Nipah virus viral protein, a Chikungunya virus viral protein, a Hendra virus viral protein, a hepatitis B virus viral protein, a hepatitis C virus viral protein, a measles virus viral protein, a Rabies virus viral protein, a respiratory syncytial virus (RSV) viral protein, a Zika virus viral protein, a Dengue virus viral protein, or a Herpes virus viral protein.
6. The method of embodiment 5, wherein the CoV viral protein includes a Wuhan CoV (COVID) viral protein, a severe acute respiratory syndrome CoV (SARS-CoV) viral protein or a Middle East respiratory syndrome coronavirus (MERS-CoV) viral protein.
7. The method of embodiment 4, wherein the protein of interest includes a viral entry protein.
8. The method of any of embodiments 4-6, wherein the viral protein is a subunit of a viral entry protein.
9. The method of embodiments 7 or 8, wherein the viral entry protein includes Chikungunya virus E1 Env or E2 Env; the Ebola glycoprotein (EBOV GP), the Hendra virus F glycoprotein or G glycoprotein; the hepatitis B virus large (L), middle (M), or small (S) protein; the hepatitis C virus glycoprotein E1 or glycoprotein E2; the HIV envelope (Env) protein; the influenza virus hemagglutinin (HA) protein, the Lassa virus envelope glycoprotein (GPC); the measles virus hemagglutinin glycoprotein (H) or fusion glycoprotein F0 (F)); the MERS-CoV Spike (S) protein; the Nipah virus fusion glycoprotein F0 (F) or glycoprotein G); the Rabies virus glycoprotein (RABV G); the RSV fusion glycoprotein F0 (F) or glycoprotein G); or the SARS-CoV Spike (S) protein. 10. The method of embodiment 8, wherein the subunit of the viral entry protein includes HIV gp41 and/or gp120.
11. The method of any of embodiments 4-10, wherein the protein of interest includes BF520.W14.C2; BG505.W6M.C2.T332N; BG505 SOSIP Env trimer; BL035.W6M.ENV.C1; SF162; ZM109F.PB4; C2-94UG114; SIV/mac239; resurfaced Env core protein (RSC3); CD4-binding site defective mutant (RSC3 Δ371I); 2J9C-ZM53_V1V2; a 1FD6-Fc-ZM109_V1V2 scaffold peptide; a V3 consensus peptide of ConA1 and ConB; MN gp41 monomer; ectodomain ZA.1197/MB; Q23 (AF004855.1); QA013.70I.Env.H1 (FJ866134); QA013.385M.Env.R3 677 (FJ396015); QB850.73P.C14; QB850.632P.B10; Q461.D1 (AF407155); or QC406.F3 (FJ866133).
12. The method of embodiment 4, wherein the protein of interest includes a bacterial protein derived from anthrax, gram-negative bacilli, chlamydia, diptheria, Helicobacter pylori, Mycobacterium tuberculosis, pertussis toxin, pneumococcus, rickettsiae, staphylococcus, streptococcus or tetanus.
13. The method of embodiment 4, wherein the protein of interest includes anthrax protective antigen, lipopolysaccharides, diptheria toxin, mycolic acid, heat shock protein 65 (HSP65), the 30 kDa major secreted protein, antigen 85A, hemagglutinin, pertactin, FIM2, FIM3, adenylate cyclase, pneumolysin, pneumococcal capsular polysaccharides, rompA, M proteins or tetanus toxin.
14. The method of embodiment 4, wherein the protein of interest includes a fungal protein derived from candida, coccidiodes, cryptococcus, histoplasma, leishmania, plasmodium, protozoa, parasites, schistosomae, tinea, toxoplasma, or Trypanosoma cruzi.
15. The method of embodiment 4, wherein the protein of interest includes spherule antigens, capsular polysaccharides, heat shock protein 60 (HSP60), gp63, lipophosphoglycan, merozoite surface antigens, sporozoite surface antigens, circumsporozoite antigens, gametocyte/gamete surface antigens, the blood-stage antigen pf 155/RESA, glutathione-S-transferase, paramyosin, trichophytin, SAG-1, p30, or the Trypanosoma cruzi 75-77 kDa antigen or the Trypanosoma cruzi 56 kDa antigen.
16. The method of embodiment 4, wherein the protein of interest includes a cancer antigen protein derived from, for example, brain cancer, breast cancer, colon cancer, HBV-induced hepatocellular carcinoma, intestinal cancer, kidney cancer, leukemia, liver cancer, lung cancer, lymphoma, melanoma, mesothelioma, multiple myeloma, ovarian cancer, pancreatic cancer, prostate cancer, renal cell carcinoma, stem cell cancer, stomach cancer, throat cancer, or uterine cancer.
17. The method of any of embodiments 4-16, wherein the protein of interest includes A33, β-catenin, BAGE, Bcl-2, BCMA, c-Met, CA19-9, CA125, CAIX, CD5, CD19, CD20, CD21, CD22, CD24, CD33, CD37, CD45, CD123, CD133, CEA, CS-1, cyclin B1, DAGE, EBNA, EGFR, ephrinB2, ERBB2, estrogen receptor, FAP, ferritin, folate-binding protein, GAGE, G250, GD2, GM2, gp75, gp100 (Pmel 17), HER-2/neu, HPV E6, HPV E7, Ki-67, LRP, mesothelin, p53, PRAME, progesterone receptor, PSA, PSCA, PSMA, MAGE, MART, mesothelin, MUC, MUM-1-B, myc, NYESO-1, ras, RORI, SV40 T, survivin, tenascin, TSTA tyrosinase, VEGF, or WT1 18. The method of any of embodiment 4-17, wherein the DMS proteins or peptides within the DMS library for the protein of interest substitute at least 95% of amino acid residues of the protein of interest with at least 17 amino acid substitutions.
19. The method of any of embodiments 4-18, wherein the DMS proteins or peptides within the DMS library for the protein of interest substitute all amino acid residues of the protein of interest with 19 amino acid substitutions.
20. The method of any of embodiments 4-19, wherein the DMS peptides are staggered fragments of the protein of interest.
21. The method of embodiment 20, wherein the staggered fragments are formed by moving 1-3 amino acid residue position down the length of the protein of interest while maintaining the same length of peptide fragments.
22. The method of embodiments 20 or 21, wherein the staggered fragments are formed by moving 1 amino acid residue position down the length of the protein of interest while maintaining the same length of peptide fragments.
23. The method of any of embodiments 1-22, wherein the DMS peptides are 50 amino acids or fewer in length.
24. The method of any of embodiments 20-23, wherein the staggered fragments are 28-33 amino acid residues in length.
25. The method of any of embodiments 1-24, wherein the DMS proteins or peptides are not barcoded.
26. The method of any of embodiments 1-25, wherein DMS proteins or peptides further include a functional sequence.
27. The method of embodiment 26, wherein the functional sequence is selected from a transport sequence, a buffer sequence, a tag sequence, and/or a selectable marker.
28. The method of embodiment 27, wherein the functional sequence includes a transport sequence.
29. The method of embodiment 28, wherein the transport sequence includes a minor coat protein, a major coat protein, a gene 10 protein, or a capsid D protein.
30. The method of embodiment 27, wherein the functional sequence includes a buffer sequence.
31. The method of embodiment 30, wherein the buffer sequence includes a flexible linker.
32. The method of embodiment 31, wherein the flexible linker is a (Gly)n (SEQ ID NO: 75), (Ser)n, (SEQ ID NO: 76), or (Ala)n (SEQ ID NO: 77) flexible linker wherein =4 or more.
33. The method of embodiment 31, wherein the flexible linker is a Gly-Ser linker or a Gly-Ala linker.
34. The method of embodiment 33, wherein the Gly-Ser linker is selected from the group including of (Gly4Ser)3 (SEQ ID NO: 74), (Gly-Ser)n (SEQ ID NO: 78), (Gly-Ser-Ser-Gly)n (SEQ ID NO: 79), (Gly-Ser-Gly)n (SEQ ID NO: 80), (Gly-Ser-Ser)n (SEQ ID NO: 81), or any combination thereof, where n=1, 2, 3, 4, 5, 6, 7, 8, 9, or 10.
35. The method of embodiments 33 or 34, wherein the Gly-Ser linker is (Gly4Ser)3 (SEQ ID NO: 74).
36. The method of any of embodiments 1-35, wherein the candidate binding molecule includes an antibody, ligand, peptide, peptide aptamer, enzyme substrate, or receptor.
37. The method of embodiment 36, wherein the candidate binding molecule includes an antibody.
38. The method of embodiment 37, wherein the antibody includes a human, mammalian, camelid, or shark antibody.
39. The method of embodiments 37 or 38, wherein the antibody includes an antibody that binds gp41.
40. The method of any of embodiments 37-39, wherein the antibody that binds gp41 includes a monoclonal antibody selected from QA255.006, QA255.016, QA255.167, QA255.072, and QA255.221.
41. The method of embodiments 37 or 38, wherein the antibody includes an antibody that binds gp120.
42. The method of embodiment 41, wherein the antibody that binds gp120 is selected from QA255.105 and QA255.157.
43. The method of embodiment 37, wherein the antibody includes VRC01, PG9, PGT121, 4E10, 50-69, 240-D, 246-D, 5F3, 2F5, 167-D, F240, D5, leronlimab, PRO 542, ibalizumab, b12, PEHRG214, 3BNC117, 131-2G, 12G5, MAB8582, MAB8581, MCA490, 104E5, 38F10, 14G3, 90D3, 56E11, 69F6, c13C6, c2G4, c4G7, c1H3, LCA60, REGN3051, REGN3048, 37.2D, 8.9F, 19.7E, 37.7H, 12.1F, or m102.4.
44. The method of embodiments 37 or 43, wherein the antibody includes leronlimab, PRO 542, ibalizumab, clone 131-2G, clone 12G5, MAB8582, MAB8581, MCA490, 104E5, 38F10, 14G3, 90D3, 56E11, 69F6, c13C6, c2G4, c4G7, c1H3, LCA60, REGN3051, REGN3048, 37.2D, 8.9F, 19.7E, 37.7H, 12.1F, m102.4, or mAb Fi6_v3.
45. The method of any of embodiments 1-44, wherein the phage include filamentous phage or bacteriophage.
46. The method of any of embodiments 1-45, wherein the phage include f1, fd, M13, T7, T4, or lambdoid phage.
47. The method of any of embodiments 1-46, further including cloning nucleotides encoding the DMS proteins or peptides into phage to create the phage library.
48. The method of any of embodiments 1-47, further including validating the phage library by sequencing.
49. The method of any of embodiments 1-48, wherein the incubating occurs within a single tube or well.
50. The method of any of embodiments 1-49, wherein the separating using immunoprecipitation includes adding magnetic beads with binding domains that bind a complex of a phage bound to the candidate binding molecule to the solution and utilizing a source of magnetism to isolate the magnetic beads.
51. The method of any of embodiments 1-50, wherein the sequencing includes next-generation sequencing (NGS).
52. The method of embodiment 51, wherein the NGS includes automated Sanger sequencing, sequencing by synthesis, pyrosequencing, sequencing by ligation, rolling amplification sequencing, single molecule sequencing, or nanopore sequencing.
53. The method of any of embodiments 1-52 wherein the determining residues responsible for the binding or non-binding of phage to the candidate binding molecule based on the sequencing includes determining an enrichment of each DMS proteins or peptide across all samples and a reproducibility threshold.
54. The method of embodiment 53, further including classifying each DMS proteins or peptide that is enriched above the reproducibility threshold as a hit within a bioinformatics analysis.
55. The method of embodiment 54, further including aligning the DMS proteins or peptides classified as hits.
56. A kit for performing protein residue mapping including a phage library expressing deep mutational scanning (DMS) proteins or peptides.
57. The kit of embodiment 56, wherein the phage include filamentous phage or bacteriophage.
58. The kit of embodiments 56 or 57, wherein the phage include f1, fd, M13, T7, T4, or lambdoid phage.
59. The kit of any of embodiments 56-58, further including magnetic beads associated with a binding domain.
60. The kit of any of embodiments 56-59, further including a candidate binding molecule.
61. The kit of embodiment 60, wherein the candidate binding molecule includes an antibody, ligand, peptide, peptide aptamer, enzyme substrate, or receptor.
62. The kit of embodiments 60 or 61, wherein the candidate binding molecule includes an antibody.
63. The kit of embodiment 62, wherein the antibody includes an antibody that binds gp120 or gp41.
64. The kit of embodiments 62 or 63, wherein the antibody includes QA255.006, QA255.016, QA255.167, QA255.072, QA255.221, QA255.105, QA255.157, VRC01, PG9, PGT121, 4E10, 50-69, 240-D, 246-D, 5F3, 2F5, 167-D, F240, D5, leronlimab, PRO 542, ibalizumab, b12, PEHRG214, 3BNC117, 131-2G, 12G5, MAB8582, MAB8581, MCA490, 104E5, 38F10, 14G3, 90D3, 56E11, 69F6, c13C6, c2G4, c4G7, c1H3, LCA60, REGN3051, REGN3048, 37.2D, 8.9F, 19.7E, 37.7H, 12.1F, m102.4, leronlimab, PRO 542, ibalizumab, clone 131-2G, clone 12G5, MAB8582, MAB8581, MCA490, 104E5, 38F10, 14G3, 90D3, 56E11, 69F6, c13C6, c2G4, c4G7, c1H3, LCA60, REGN3051, REGN3048, 37.2D, 8.9F, 19.7E, 37.7H, 12.1F, m102.4, or mAb Fi6_v3.
65. The kit of any of embodiments 56-64, wherein the DMS proteins or peptides are derived from a protein of interest selected from a viral protein, a bacterial protein, a fungal protein, or a cancer cell antigen.
66. The kit of embodiment 65, wherein the viral protein includes a human immunodeficiency virus-1 (HIV-1) viral protein, an HIV-2 viral protein, a simian immunodeficiency virus (SIV) viral protein, an influenza virus viral protein, an Ebola virus viral protein, a coronavirus (CoV) viral protein, a Lassa virus viral protein, a Nipah virus viral protein, a Chikungunya virus viral protein, a Hendra virus viral protein, a hepatitis B virus viral protein, a hepatitis C virus viral protein, a measles virus viral protein, a Rabies virus viral protein, a respiratory syncytial virus (RSV) viral protein, a Zika virus viral protein, a Dengue virus viral protein, or a Herpes virus viral protein.
67. The kit of embodiment 66, wherein the CoV viral protein includes a Wuhan CoV (COVID) viral protein, a severe acute respiratory syndrome CoV (SARS-CoV) viral protein or a Middle East respiratory syndrome coronavirus (MERS-CoV) viral protein.
68. The kit of embodiment 65, wherein the protein of interest includes a viral entry protein.
69. The kit of embodiment 65, wherein the viral protein is a subunit of a viral entry protein.
70. The kit of embodiment 69, wherein the viral entry protein includes Chikungunya virus E1 Env or E2 Env; the Ebola glycoprotein (EBOV GP), the Hendra virus F glycoprotein or G glycoprotein; the hepatitis B virus large (L), middle (M), or small (S) protein; the hepatitis C virus glycoprotein E1 or glycoprotein E2; the HIV envelope (Env) protein; the influenza virus hemagglutinin (HA) protein, the Lassa virus envelope glycoprotein (GPC); the measles virus hemagglutinin glycoprotein (H) or fusion glycoprotein F0 (F)); the MERS-CoV Spike (S) protein; the Nipah virus fusion glycoprotein F0 (F) or glycoprotein G); the Rabies virus glycoprotein (RABV G); the RSV fusion glycoprotein F0 (F) or glycoprotein G); or the SARS-CoV Spike (S) protein.
71. The kit of embodiment 69, wherein the subunit of the viral entry protein includes HIV gp41 and/or gp120.
72. The kit of embodiment 65, wherein the protein of interest includes BF520.W14.C2; BG505.W6M.C2.T332N; BG505 SOSIP Env trimer; BL035.W6M.ENV.C1; SF162; ZM109F.PB4; C2-94UG114; SIV/mac239; resurfaced Env core protein (RSC3); CD4-binding site defective mutant; 2J9C-ZM53_V1V2; a 1FD6-Fc-ZM109_V1V2 scaffold peptide; a V3 consensus peptide of ConA1 and ConB; MN gp41 monomer; ectodomain ZA.1197/MB; Q23; QA013.70I.Env.H1; QA013.385M.Env.R3 677; QB850.73P.C14; QB850.632P.B10; Q461.D1; or QC406.F3.
73. The kit of embodiment 65, wherein the protein of interest includes a bacterial protein derived from anthrax, gram-negative bacilli, chlamydia, diptheria, Helicobacter pylori, Mycobacterium tuberculosis, pertussis toxin, pneumococcus, rickettsiae, staphylococcus, streptococcus or tetanus.
74. The kit of embodiment 65, wherein the protein of interest includes anthrax protective antigen, lipopolysaccharides, diptheria toxin, mycolic acid, heat shock protein 65 (HSP65), the 30 kDa major secreted protein, antigen 85A, hemagglutinin, pertactin, FIM2, FIM3, adenylate cyclase, pneumolysin, pneumococcal capsular polysaccharides, rompA, M proteins or tetanus toxin.
75. The kit of embodiment 65, wherein the protein of interest includes a fungal protein derived from candida, coccidiodes, cryptococcus, histoplasma, leishmania, plasmodium, protozoa, parasites, schistosomae, tinea, toxoplasma, or Trypanosoma cruzi.
76. The kit of embodiment 65, wherein the protein of interest includes spherule antigens, capsular polysaccharides, heat shock protein 60 (HSP60), gp63, lipophosphoglycan, merozoite surface antigens, sporozoite surface antigens, circumsporozoite antigens, gametocyte/gamete surface antigens, the blood-stage antigen pf 155/RESA, glutathione-S-transferase, paramyosin, trichophytin, SAG-1, p30, or the Trypanosoma cruzi 75-77 kDa antigen or the Trypanosoma cruzi 56 kDa antigen.
77. The kit of embodiment 65, wherein the protein of interest includes a cancer antigen protein derived from, for example, brain cancer, breast cancer, colon cancer, H BV-induced hepatocellular carcinoma, intestinal cancer, kidney cancer, leukemia, liver cancer, lung cancer, lymphoma, melanoma, mesothelioma, multiple myeloma, ovarian cancer, pancreatic cancer, prostate cancer, renal cell carcinoma, stem cell cancer, stomach cancer, throat cancer, or uterine cancer.
78. The kit of any of embodiments 65-77, wherein the protein of interest includes A33, β-catenin, BAGE, Bcl-2, BCMA, c-Met, CA19-9, CA125, CAIX, CD5, CD19, CD20, CD21, CD22, CD24, CD33, CD37, CD45, CD123, CD133, CEA, CS-1, cyclin 1, DAGE, EBNA, EGFR, ephrinB2, ERBB2, estrogen receptor, FAP, ferritin, folate-binding protein, GAGE, G250, GD2, GM2, gp75, gp100 (Pmel 17), HER-2/neu, HPV E6, HPV E7, Ki-67, LRP, mesothelin, p53, PRAME, progesterone receptor, PSA, PSCA, PSMA, MAGE, MART, mesothelin, MUC, MUM-1-B, myc, NYESO-1, ras, RORI, SV40 T, survivin, tenascin, TSTA tyrosinase, VEGF, or WT1 79. The kit of any of embodiments 56-78, wherein the DMS proteins or peptides within the DMS library for the protein of interest substitute at least 95% of amino acid residues of the protein of interest with at least 17 amino acid substitutions.
80. The kit of any of embodiments 56-79, wherein the DMS proteins or peptides within the DMS library for the protein of interest substitute all amino acid residues of the protein of interest with 19 amino acid substitutions.
81. The kit of any of embodiments 56-80, wherein the DMS peptides are staggered fragments of the protein of interest.
82. The kit of embodiment 81, wherein the staggered fragments are formed by moving 1-3 amino acid residue position down the length of the protein of interest while maintaining the same length of peptide fragments.
83. The kit of embodiments 81 or 82, wherein the staggered fragments are formed by moving 1 amino acid residue position down the length of the protein of interest while maintaining the same length of peptide fragments.
84. The kit of any of embodiment 56-83, wherein the DMS peptides are 50 amino acids or fewer in length.
85. The kit of embodiments 81-84, wherein the staggered fragments are 28-33 amino acid residues in length.
86. The kit of any of embodiments 56-85, wherein the DMS proteins or peptides are not barcoded.
87. The kit of any of embodiments 56-86, wherein DMS proteins or peptides further include a functional sequence.
88. The kit of embodiment 87, wherein the functional sequence is selected from a transport sequence, a buffer sequence, a tag sequence, and/or a selectable marker.
89. The kit of embodiments 87 or 88, wherein the functional sequence includes a transport sequence.
90. The kit of embodiment 89, wherein the transport sequence includes a minor coat protein, a major coat protein, a gene 10 protein, or a capsid D protein.
91. The kit of embodiments 87 or 88, wherein the functional sequence includes a buffer sequence.
92. The kit of embodiment 91, wherein the buffer sequence includes a flexible linker.
93. The kit of embodiment 92, wherein the flexible linker is a (Gly)n (SEQ ID NO: 75), (Ser)n, (SEQ ID NO: 76), or (Ala)n (SEQ ID NO: 77) flexible linker wherein =4 or more.
94. The kit of embodiment 92, wherein the flexible linker is a Gly-Ser linker or a Gly-Ala linker.
95. The kit of embodiment 94, wherein the Gly-Ser linker is selected from the group including of (Gly4Ser)3 (SEQ ID NO: 74), (Gly-Ser)n (SEQ ID NO: 78), (Gly-Ser-Ser-Gly)n (SEQ ID NO: 79), (Gly-Ser-Gly)n (SEQ ID NO: 80), (Gly-Ser-Ser)n (SEQ ID NO: 81), or any combination thereof, where n=1, 2, 3, 4, 5, 6, 7, 8, 9, or 10.
96. The kit of embodiments 94 or 95, wherein the Gly-Ser linker is (Gly4Ser)3 (SEQ ID NO: 74).

(vii) Experimental Examples. Example 1. Monoclonal antibodies that target HIV transmembrane protein gp41 and mediate killing of HIV-infected cells through antibody-dependent cellular cytotoxicity.

Anti-HIV antibodies can mediate activity by neutralizing cell-free virus or binding to infected cells and driving antibody-dependent cellular cytotoxicity (ADCC). While numerous discovery efforts have identified and characterized neutralizing antibodies, much less is known about antibodies that mediate ADCC. Four new antibodies that target the gp41 transmembrane protein of the HIV envelope are disclosed. Competition experiments and peptide mapping studies together helped narrow down the binding sites for the four antibodies to two conserved regions of the protein. One pair of antibodies targets a common epitope of gp41 while the other pair binds to a more complex discontinuous epitope. In vitro activity assays indicated that this second pair of antibodies could drive killing against cells coated with various forms of gp41, and both pairs of antibodies could drive killing of HIV-infected cells. Inducing these types of antibodies following vaccination may represent a more straightforward path to generating a consistent, functional response to a more conserved portion of the HIV envelope protein.

Eliciting an antibody response to the HIV Envelope protein is thought to be the most likely path to an effective vaccine, and there is evidence that both neutralizing and non-neutralizing HIV-specific antibodies can contribute to protection. Indeed, the only HIV vaccine trial to demonstrate measurable protection from HIV infection implicated non-neutralizing antibodies capable of mediating antibody-dependent cellular cytotoxicity (ADCC) (Haynes et al. N Engl J Med. 366:1275-1286, 2012). Studies of mother-infant HIV transmission, a setting where both maternal antibodies and antibodies passively acquired by infants in utero are present during the period of transmission risk, have similarly implicated ADCC antibodies in protection. Specifically, ADCC-mediating antibodies isolated from breastmilk were correlated with infant infection outcome in women with high viral load (Mabuka et al. PLoS Pathog. 8:e1002739, 2012), and passively acquired ADCC-mediating antibodies correlated with clinical outcome in infants who acquired HIV after birth (Milligan et al. Cell Host Microbe. 2015; 17:500-506). Evidence from studies in non-human primate models have similarly supported a role for non-neutralizing ADCC-mediating antibodies in limiting disease pathogenesis (Alpert et al. PLoS Pathog. 8:e1002890, 2012; Banks et al., AIDS Res Hum Retroviruses. 18:1197-1205, 2002; Barouch et al. Science. 2015; 349:320-324; Barouch et al. Nature. 2012; 482:89-93; Burton et al. Proc Natl Acad Sci USA. 2011; 108:11181-11186; Fouts et al. Proc Natl Acad Sci USA. 2015; 112:E992-E999; Gomez-Roman et al. J Immunol. 2005; 174:2185-2189; Hidajat et al. J Virol. 2009; 83:791-801; Lewis et al. Immunol Rev. 2017; 275:271-284; Moog et al. Mucosal Immunol. 2014; 7:46-56; Sun et al. J Virol. 2011; 85:6906-6912; Thomas et al. Virology. 2014; 471-473:81-92; Xiao et al. J Virol. 2012; 86:4644-4657; Xiao et al. J Virol. 2010; 84:7161-7173), and antibodies defective in Fc-receptor binding demonstrated reduced protective efficacy (Hessell et al. Nat Med. 2009; 15:951-954; Hessell et al. Nature. 2007; 449:101-104). Further investigation into the epitope targets of ADCC-mediating mAbs and their contribution to protection may help inform future vaccine strategies.

Most studies have focused on antibodies directed to gp120, the extracellular Env glycoprotein. The envelope transmembrane protein, gp41, which is required for viral entry, is also a target of both neutralizing and non-neutralizing HIV antibodies (Gallerano et al. Int Arch Allergy Immunol. 2015; 167:223-241; Gorny et al. HIV Immunol and HIV/SIV Vac Databases. 2003:37-51; Montero et al. Microbiol Mol Biol Rev. 2008; 72:54-84; Pollara et al. Curr HIV Res. 2013; 11:378-387; Wu et al. Curr Opin Immunol. 2016; 42:56-64). During the entry process, gp41 undergoes a series of conformational changes that drive viral and host cell membrane fusion, resulting in opportunities for antibodies to recognize different gp41 epitopes at various stages in the process. Gp41 encodes several key functional domains in its extracellular portion (ectodomain) where antibodies target. These include the fusion peptide, which becomes exposed as a result of structural changes that promote fusion. There are also two heptad repeat (HR) regions (N terminal HR/NHR and C terminal HR/CHR) that are separated by a disulfide-bonded loop (C-C′ loop), which presents an immunodominant epitope. The interaction of the NHR and CHR during the entry process leads to a six-helix bundle structure that joins the viral and cell membranes together. The region at the C-terminus of the extracellular domain of gp41, the membrane proximal region (MPER), is a target of several broadly neutralizing antibodies (Montero et al. Microbiol Mol Biol Rev. 2008; 72:54-84; Wu et al. Curr Opin Immunol. 2016; 42:56-64). Because the extracellular regions of gp41 are conserved, gp41 is an excellent target for cross-reactive antibodies recognizing diverse viral strains (Steckbeck et al. J Biol Chem. 2011; 286:27156-27166). Further, as virus buds from infected cells, some gp120 protein are shed. As a result, gp41 stumps are exposed on the cell surface (Moore et al. J Virol. 2006; 80:2515-2528) and can be targeted by gp41-specific, ADCC-mediating antibodies (Moog et al. Mucosal Immunol. 2014; 7:46-56; Pollara et al. Curr HIV Res. 2013; 11:378-387; Evans et al. AIDS. 1989; 3:273-276; Forthal et al. AIDS Res Hum Retroviruses. 1995; 11:1095-1099; Tyler et al. AIDS Res Hum Retroviruses. 1989; 5:557-563; Tyler et al. J Immunol. 1990; 144:3375-3384; Tyler et al. J Immunol. 1990; 145:3276-3282).

Env gp41-directed antibodies arise early in infection (Tomaras et al. J Virol. 2008; 82:12449-12463) and several common targets have been described, including antibodies that recognize the C-C′ loop, which encodes an immunodominant epitope of gp41 (referred to as cluster I antibodies) and others that recognize the CHR (cluster II antibodies), with cluster I being common in chronic infection (Gorny HIV Immunol and HIV/SIV Vac Databases. 2003:37-51; Alsmadi et al. J Virol. 1998; 72:286-293; Buchacher et al. AIDS Res Hum Retroviruses. 1994; 10:359-369; Corti et al. PLoS One 2010; 5:e8805; Gnann et al. J Infect Dis. 1987; 156:261-267; Gorny et al. Virology. 2000; 267:220-228; Pietzsch et al. J Virol. 2010; 84:5032-5042; Santra et al. PLOS Pathog. 2015; 11:e1005042; Xu et al. J Virol. 1991; 65:4832-4838) and associated with a broad response (Santra et al. PLOS Pathog. 2015; 11:e1005042; Burrer et al. Virology. 2005; 333:102-113; Cavacini et al. AIDS Res Hum Retroviruses. 1998; 14:1271-1280; Nyambi et al. J Virol. 2000; 74:7096-7107). Anti-cluster I antibodies inhibit HIV via a variety of mechanisms (Burton et al. Proc Natl Acad Sci USA. 2011; 108:11181-11186; Moog et al. Mucosal Immunol. 2014; 7:46-56; Santra et al. PLOS Pathog. 2015; 11:e1005042; Holl et al. J Virol. 2006; 80:6177-6181; Horwitz et al. Cell. 2017; 170:637-648.e610; Peressin et al. J Virol. 2011; 85:1077-1085; Shen et al. J Immunol. 2010; 184:3648-3655; Spear et al. J Virol. 1993; 67:53-59; Neurath et al. J Gen Virol. 1990; 71: 85-95), including neutralization and ADCC (Moog et al. Mucosal Immunol. 2014; 7:46-56; Montero et al. Microbiol Mol Biol Rev. 2008; 72:54-84; Wu et al. Curr Opin Immunol. 2016; 42:56-64; Forthal et al. AIDS Res Hum Retroviruses. 1995; 11:1095-1099; Tyler et al. J Immunol. 1990; 145:3276-3282; Alsmadi et al. J Virol. 1998; 72:286-293; Pietzsch et al. J Virol. 2010; 84:5032-5042; Santra et al. PLOS Pathog. 2015; 11:e1005042), though gp41-specific ADCC-mediating antibodies have been less well studied than neutralizing antibodies. However, there is evidence that ADCC antibodies could provide protection in both model systems and humans. IgA gp41-targeting antibodies have been isolated from highly exposed, HIV-negative individuals (Belec et al. J Infect Dis. 2001; 184:1412-1422; Lopalco et al. J Gen Virol. 2005; 86:339-348; Nguyen et al. J Acquir Immune Defic Syndr. 2006; 42:412-419; Tudor et al. Mucosal Immunol. 2009; 2:412-426) and associated with protection (Benjelloun et al. AIDS. 2013; 27:1992-1995; Clerici et al. AIDS. 2002; 16:1731-1741; Kaul et al. AIDS. 2001; 15:431-432; Pastori et al., J Biol Regul Homeost Agents. 2000; 14:15-21). Moreover, a gp41-based antigen elicited protection in a macaque model of mucosal infection (Bomsel et al. Immunity. 2011; 34:269-280). Studies investigating the anti-viral effects of passively administered ADCC-mediating antibodies, while few relative to the plethora of passive neutralizing antibody studies, also provide some evidence for a non-sterilizing protective effect of gp41 antibodies (Burton et al. Proc Natl Acad Sci USA. 2011; 108:11181-11186; Lewis et al. Immunol Rev. 2017; 275:271-284; Santra et al. PLOS Pathog. 2015; 11:e1005042; Horwitz et al. Cell. 2017; 170:637-648.e610; Forthal et al. Curr Opin HIV AIDS. 2009; 4:388-393; Hessell et al. J Virol. 2010; 84:1302-1313; Lewis et al. Viruses. 2015; 7:5115-5132; Lewis et al. Curr HIV Res. 2013; 11:354-364; Lewis et al. Curr Opin HIV AIDS. 2016; 11:561-568; Lewis et al. Curr Opin HIV AIDS. 2014; 9:263-270), and in particular, an effect of cluster I ADCC-mediating antibodies on viral load (Moog et al. Mucosal Immunol. 2014; 7:46-56).

Monoclonal antibodies (mAbs) from a clade A-infected individual were isolated by selecting B cells that bound to HIV virus-like particles (VLPs) (Williams et al. EBioMedicine. 2015; 2:1464-1477). While some of the reconstructed mAbs recognized gp120, others did not, even though they showed detectable binding to the VLPs used as bait. One such antibody showed evidence of antibody-dependent cellular viral inhibition (ADCVI) activity (Williams et al. EBioMedicine. 2015; 2:1464-1477), prompting further evaluation of the HIV-specific mAbs from this individual that did not recognize gp120. This Example shows that several of the VLP-specific antibodies target gp41 and mediate ADCC, including the antibody that demonstrated ADCVI activity. The four mAbs identified in this one individual all arose from independent B cell lineages and target either the immunodominant epitope that defines cluster I or a discontinuous epitope. A unique phage display approach to more finely map the epitopes of the two gp41 cluster I antibodies was used and showed that they have overlapping but distinct epitopes. The two other mAbs both target a similar discontinuous conformational epitope that includes both the CHR and the FPPR portions of gp41. These mAbs also recognize a structure that mimics gp41 stumps and drive ADCC activity against cells coated with this gp41 mimetic.

Methods. QA255 antibody synthesis. Antibodies from QA255 were originally isolated and cloned as described previously (Williams et al. EBioMedicine. 2015; 2:1464-1477). In brief, paired heavy and light chain DNA clones were co-transfected in equal ratios into 293F cells (293 Freestyle cells; Thermo Fisher, Waltham, Mass.; 1×106 cells/1 μg of total DNA) with a 16:1:1 (OptiPRO Serum-Free Medium:293Max:DNA, Thermo Fisher, Waltham, Mass.) ratio. Antibodies were harvested after 72 h and purified using Protein G resin in hand-packed, gravity flow columns (Pierce). Antibody concentration was determined using protein absorbance at 280 nM (Nanodrop, Thermo Fisher, Waltham, Mass.).

Binding Antibody Multiplex Assay (BAMA). The BAMA was conducted as described (Haynes et al. N Engl J Med. 2012; 366:1275-1286; Tomaras et al. J Virol. 2008; 82:12449-12463; Mayr et al. Sci Rep. 2017; 7:12655; McLean et al. J. Immunol. 2017; 199:816-826) to measure IgG binding to a panel of HIV antigens. Prior to performing the BAMA, antigens were covalently conjugated to carboxylated fluorescent beads (Luminex) as described previously (Tomaras et al. J Virol. 2008; 82:12449-12463; Ramirez Valdez et al. Virology. 2015; 475:187-203). Antigen-conjugated beads were stored in PBS (Gibco) containing 0.1% bovine serum albumin (BSA; Sigma-Aldrich), 0.02% Tween™ (Sigma-Aldrich), and 0.05% sodium azide (Sigma-Aldrich) at the optimal temperature for the unconjugated antigen for up to 1 year. Antigens included in the assay were monomeric gp120 proteins BG505.W6M.C2.T332N (clade A), BL035.W6M.ENV.C1 (clade A/D recombinant), SF162 (clade B), ZM109F.PB4 (clade C), C2-94UG114 (clade D), and SIV/mac239; clade A BG505 SOSIP Env trimer (Sanders et al. PLoS Pathog. 2013; 9:e1003618); resurfaced Env core protein (RSC3) and CD4-binding site defective mutant (RSC3 Δ371I) (construct obtained from NIH AIDS Reagent Program, Division of AIDS, NIAID, NIH) and produced as described in (Cortez et al. PLoS Pathog. 2015; 11:e1004973); clade C 2J9C-ZM53_V1V2 and 1FD6-Fc-ZM109_V1V2 scaffolded peptides (Jiang et al. J Virol. 2016; 90:11007-11019); V3 consensus peptides ConA1 (CTRPNNNTRKSIRIGPGQAFYATGDIIGDIRQAHC, SEQ ID NO: 89) and ConB (CTRPNNNTRKSIHIGPGRAFYTTGEIIGDIRQAHC, SEQ ID NO: 90) (Genscript); and two gp41 antigens: clade B MN gp41 monomer (NIH AIDS Reagent Program, Division of AIDS, NIAID, NIH from ImmunoDX, LLC) and clade C ectodomain ZA.1197/MB (Immune Technology Corp). BG505 gp120 was produced by transient transfection of 293F cells (Thermo Fisher, Waltham, Mass.) followed by Galanthus nivalis lectin purification (Vector Laboratories) as described previously (Verkerke et al. J Virol. 2016; 90:9471-9482). All other gp120 proteins were purchased from Immune Tech. Positive controls included VRC01, PG9, PGT121, 4E10, 50-69, and 246-D. VRC01, PG9 and PGT121 were all produced as described above and 4E10, 50-69, and 246-D obtained from the NIH AIDS Reagent Program, Division of AIDS, NIAID, NIH (4E10 from Polymun Scientific, and 50-69 and 246-D from Dr. Susan Zolla-Pazner). Negative controls included both HIV-negative plasma and mock conjugated beads. Binding was measured as the mean fluorescence intensity (MFI) and averaged across duplicate wells. Results are reported as fold change over binding by HIV-negative plasma.

gp41 Binding ELISA. The gp41 binding ELISA was adapted from (Williams et al. EBioMedicine. 2015; 2:1464-1477). In brief, Immunolon 2-HB plates were coated with 100 μL of MN gp41 (NIH AIDS Reagent Program, Division of AIDS, NIAID, NIH from ImmunoDX, LLC.) or ZA.1197 (Immune Technology Corp) at 0.5 μg/mL in 0.1M sodium bicarbonate coating buffer (pH 7.4) overnight at 4° C. Plates were rinsed 4-5 times using PBS-0.05% Tween™ wash buffer. Plates were blocked with 10% non-fat dry milk (NFDM) diluted into wash buffer for at least 1 h. After removing the blocking buffer, 100 μL of primary mAb diluted in blocking buffer was added and incubated at 37° C. for 1 h. Plates were washed a second time and 100 μL of anti-IgG-HRP (Sigma-Aldrich) diluted 1:2500 in blocking buffer was added and incubated at room temperature for 1 h. Plates were washed and 50 μL Ultra-TMB (Thermo Scientific) substrate added to each well and incubated at room temperature for 10 min. This reaction was stopped by adding 50 μL of 0.1 M H₂SO₄ (Sigma-Aldrich, St. Louis, Mo.) and the absorbance was read at 450 nM optical density within 30 min. The endpoint titers for all antibodies were defined as the average Ab concentration with binding greater than 2-fold of the negative control, Influenza-specific mAb Fi6_v3.

For ELISA assays, 96-well plates (Nunc Maxisorp™ flat-bottom, Thermo Fisher Scientific) were coated with 5 μg/mL streptavidin (in 50 mM sodium bicarbonate pH 8.75) for at least 1 h, before the addition of 5 μg/mL biotinylated 6-helix or 5-helix. Following coating with antigens, the plates were washed three times with 300 μL 1×PBST and blocked with 300 μL of 1×PBST with 0.5% BSA for at least one hour. Following blocking, antibodies were added in serial 10-fold dilutions starting at 75 μg/mL for at least 1 h. The plates were then washed 3× with 300 μL of 1×PBST and an anti-human IgG HRP secondary antibody (Thermo Fisher) was added for 1 h at room temperature. The plates were then washed 6× with 300 μL of 1×PBST and developed using 1-Step™ Turbo TMB ELISA substrate solution (Thermo Fisher Scientific) for 6 mins and quenched using 2M H2SO4. The readout of this colorimetric assay was determined using a 96 well plate reader (Biotek), and the intensity of the absorbance at 450 nm was normalized for the path length. Finally, these resulting values were baseline subtracted (subtracting the average of the background signal from secondary antibody only control wells). EC50s were obtained from fitting values to a sigmoidal curve in GraphPad Prism v7.0c.

MAb D5, which binds to the highly conserved hydrophobic pocket on the NHR (Fraser et al. Science. 2014; 343:1243727), was obtained through the NIH AIDS Reagent Program, Division of AIDS, NIAID, NIH.

Rapid and Fluorometric ADCC (RF-ADCC) assay. The RF-ADCC assay was performed as described (Mabuka et al. PLoS Pathog. 2012; 8:e1002739; Milligan et al. Cell Host Microbe. 2015; 17:500-506; Williams et al. EBioMedicine. 2015; 2:1464-1477; Gómez-Román et al. J Immunol Methods. 2006; 308:53-67). In short, CEM-NKr cells (AIDS Research and Reference Reagent Program, NIAID, NIH) were double labeled with PKH26-cell membrane dye (Sigma-Aldrich) and a cytoplasmic-staining CSFE dye (Vybrant CFDA SE Cell Tracer Kit, Life Technologies). The double-labeled cells were coated with either clade A gp140 (Q461.e2) (Blish et al. PLoS Med. 2008; 5:e9), MN gp41 (NIH AIDS Reagent Program, Division of AIDS, NIAID, NIH from ImmunoDX, LLC), or the 6-helix gp41 mimetic for 1 h at room temperature at a ratio of 1.5 μg protein (1 μg/μL):1×10⁵ double-stained target cells. Coated targets were washed once with complete RMPI media (Gibco) supplemented with 10% FBS (Gibco), 4.0 mM Glutamax (Gibco) and 1% antibiotic-antimycotic (Life Technologies). Monoclonal antibodies were diluted in complete RPMI media and mixed with 5×10³ coated target cells for 10 min at room temperature. PBMCs (peripheral blood mononuclear cells; Bloodworks Northwest) from an HIV-negative donor were then added at a ratio of 50 effector cells per target cell. The coated target cells, antibodies, and effector cells were co-cultured for 4 h at 37° C. then fixed in 150 μL 1% paraformaldehyde (Affymetrix). Cells were acquired by flow cytometry (LSR II, BD) and ADCC activity defined as the percent of PE+, FITC-cells with background subtracted where background (antibody-mediated killing of uncoated cells) was between 3-5% as analyzed using FlowJo software (Tree Star). The data were plotted with percent ADCC activity on the y-axis and respective mAb on the x-axis (Graphpad Prism v7.0c).

Competition ELISAs. Antibodies selected for the competition ELISA experiments were all obtained from the NIH AIDS Reagent Program, Division of AIDS, NIAID and included: 5F3 and 2F5; 167-D, 240-D, 50-69, and 246-D; F240; and D5.

Immunolon 2-HB plates were coated with MN gp41 as described above. Competitor antibodies were added first at a concentration of 10 μg/mL to gp41-coated plates and incubated for 15 min at 37° C. Biotinylated (BT; Thermo Fisher) QA255 antibodies were added next without washing and the competitor/BT-antibody mixture were incubated together for 45 min at 37° C. Limiting concentrations for each BT mAb were pre-determined as follows: BT-QA255.006 at 1.25 μg/mL, BT-QA255.016 at 10 μg/mL, BT-QA255.067 and BT-QA255.072 both at 0.625 μg/mL. Plates were then washed thoroughly and HRP-conjugated Streptavidin diluted in wash buffer (1:1000) added and incubated for at least 45 min. After washing, Ultra TMB-substrate and 0.1 M H₂SO₄ were added as previously described. Relative BT-mAb binding was calculated by dividing each BT-mAb binding in the presence of each competitor antibody by the average of the same BT-mAb binding in the presence of blocking buffer.

Phage Display Immunoprecipitation-Sequencing. To identify precise epitopes of antibodies in this study an approach that couples phage immunoprecipitation and highly-multiplexed sequencing was utilized (Xu et al. Science. 2015; 348:aaa0698). A phage-display library that had been designed to study febrile pathogens that are prevalent in East Africa was used. Of particular interest to this study, the library contains several full-length HIV sequences from each clade, including consensus sequences from Clades A, B, C, and D (LANL), Q23 (AF004855.1), BF520.W14M.C2 (KX168094), BG505.W6.C2 (DQ208458), and Env sequences from QA013.70I.Env.H1 (FJ866134), QA013.385M.Env.R3 677 (FJ396015), QB850.73P.C14, QB850.632P.B10, Q461.D1 (AF407155), and QC406.F3 (FJ866133).

To generate the library, 39-amino acid sequences were generated that tiled over the coding sequences of viral genomes of interest with 20-amino acid overlap. These protein sequences were reverse translated to DNA sequences and codon-optimized for expression in E. coli. Synonymous mutations were introduced to avoid EcoRI and HindIII restriction sites that were used in subsequent cloning steps. Adapter sequences (5′: AGGAATTCTACGCTGAGT (SEQ ID NO: 91) and 3′: TGATAGCAAGCTTGCC (SEQ ID NO: 92)) were added and the library was ordered on a releasable DNA microarray (Twist Biosciences). The library was then PCR amplified using T7F (AATGATACGGCAGGAATTCTACGCTGAGT, SEQ ID NO: 93) and T7R (CGATCAGCAGAGGCAAGCTTGCTATCA, SEQ ID NO: 94) primers, digested with EcoRI and HindIII, cloned into the T7Select® 10-3b Vector, and packaged into T7 phage and amplified according to the manufacturer's protocol (EMD Millipore, Burlington, Mass.).

Phage immunoprecipitation was performed as previously described (Xu et al. Science. 2015; 348:aaa0698). 96-deep-well plates (CoStar) were blocked with 3% BSA in TBST (Tris-buffered saline-Tween™) by placing on a rotator overnight at 4° C. 1 mL of amplified phage at 2×10⁵-fold representation (1.2×10⁹ pfu/mL for a library of 5.8×10³ phage) was added to each well, followed by either 2 ng or 10 ng of purified anti-gp41 monoclonal antibody. Each concentration of monoclonal antibody was tested in technical replicate. Phage-antibody complexes were formed by rotating the plate at 4° C. for 20 hours. To immunoprecipitate phage-antibody complexes, 40 μL of a 1:1 mix of protein A and protein G Dynabeads (Invitrogen, Carlsbad, Calif.) was added to each well and rotated at 4° C. for 4 hours. After this incubation, a magnetic plate was used to isolate the beads and perform 3 washes with 400 μL of wash buffer (50 mM Tris-HCl, pH 7.5, 150 mM NaCl, 0.1% NP-40). The beads were resuspended in 40 μL of water and isolated phage were lysed by incubating at 95° C. for 10 mins. Phage that did not undergo immunoprecipitation (‘input’) were also lysed to determine the starting frequencies of each phage clone in the library. Isolated phage DNA was then prepared for highly-multiplex sequencing by performing two rounds of PCR with Q5 High-Fidelity DNA polymerase (New England Biolabs, Ipswich, Mass.) to add Illumina adapters and barcodes according to the manufacturer's suggested protocol (NEB). The first-round PCR was performed with primers

### R1_F (TCGTCGGCAGCGTCTCCAGTCAGGTGTGATGCTC, SEQ ID NO: 95) and

R1_R (GTGGGCTCGGAGATGTGTATAAGAGACAGCAAGACCCGTTTAGAGGCCC, SEQ ID NO: 96). 1 μL of purified first-round product was added to the second-round PCR with unique dual indexed primers

### R2_F (AATGATACGGCGACCACCGAGATCTACACxxxxxxxxTCGTCGGCAGCGTCTCCAGTC, SEQ ID NO: 26) and

R2_R (CAAGCAGAAGACGGCATACGAGATxxxxxxxxGTCTCGTGGGCTCGGAGATGTGTATAAGAG ACAG, SEQ ID NO: 46). In these primer sequences, “xxxxxxxx” corresponds to a unique 8-nt indexing sequence. Second-round PCR products were quantified in each sample using Quant-iT PicoGreen according to the manufacturer's suggested protocol (Thermo Fisher). Equimolar quantities of each sample were then pooled, gel isolated, and submitted for Illumina sequencing on a MiSeq, where 60,000-1,100,000 reads were obtained for each sample.

Bioinformatics analyses of the sequencing data was performed as previously described (Xu et al. Science. 2015; 348:aaa0698). In brief, a zero-inflated generalized Poisson significant-enrichment assignment algorithm was used to generate a −log₁₀(p-value) for enrichment of each clone across all samples. A reproducibility threshold was established to call ‘hits’ in technical replicate pairs by first calculating the log₁₀(−log₁₀(p-value)) for each clone in Replicate 1. These values were then surveyed in Replicate 2 by using a sliding window of width 0.01 from −2 to the maximum log₁₀(−log₁₀(p-value)) value in Replicate 1. For all clones that fell within each window, the median and median absolute deviation of log₁₀(−log₁₀(p-values)) in Replicate 2 were calculated and plotted against the window location. The reproducibility threshold was set as the window location where the median was greater than the median absolute deviation. The distribution of the threshold −log₁₀(p-values) was centered around a median of 2.2. In sum, a phage clone was called a ‘hit’ if the −log₁₀(p value) was at least 2.2 in both replicates. Beads-only samples, which serve as a negative control for non-specific binding of phage, were used to identify and eliminate background hits. Peptides called as hits were aligned using Clustal Omega. The shortest amino acid sequence present in all of the hits was defined as the “minimal epitope” of an antibody. Of note, peptides were tiled as described above.

QA255 envelope cloning and sequencing. Methods describing amplification and characterization of envelope clones from PMBC DNA from 189, 560, 662 and 1729 days post-infection were previous described (Bosch et al. Virology. 2010; 398:115-124). Envelope clones from 21 days post-infection were generated from plasma RNA using similar methods. In both cases, a limiting dilution PCR strategy was used to amplify single genome envelope sequences.

Flow cytometry analysis of cell-surface staining and ADCC. For cell surface staining, infected or mock-infected CEM.NKr (CEM cells resistant to Natural Killer cells killing, Alpert et al. PLoS Pathog. 2012; 8:e1002890) were incubated for 30 min at room temperature 48 h post-infection with 5 μg/ml of each tested antibody in PBS. Cells were then washed twice with PBS and stained with 1 μg/ml of goat anti-human antibody (Alexa Fluor-647, Invitrogen) for 15 min in PBS. After two more PBS washing, cells were fixed in a 2% PBS-formaldehyde solution. ADCC was performed with a previously described assay (Veillette et al. J Virol. 2014; 88:2633-2644). Briefly, CEM.NKr infected cells were stained with viability (AquaVivid; Invitrogen) and cellular (cell proliferation dye eFluor670; eBiosciences) markers and used as target cells. Effector PBMCs, stained with another cellular marker (cell proliferation dye eFluor450; eBiosciences), were then mixed at an effector/target (E/T) ratio of 10:1 in 96-well V-bottom plates (Corning); 5 μg/ml of the desired Ab was added to appropriate wells. Co-cultures were centrifuged for 1 min at 300 g and incubated at 37° C. for 5-6 h before being fixed in a 2% PBS-formaldehyde solution containing 5×10⁴/ml flow cytometry particles (AccuCount Blank Particles, 5.3 μm; Spherotech, Lake Forest, Ill.). IFN-α (PBLAssay Science) was reconstituted in RPMI-1640 complete medium at 1×10⁷ U/mL, aliquoted, and stored at −80° C. IFN-α was then added to the cells at 1000 U/mL 24 h post-infection, 24 h before cell-surface staining or ADCC assays. Samples were analyzed on an LSRII cytometer (BD Biosciences, San Jose, Calif.) and acquisition was set to acquire 1000 particles, which allows the calculation of relative cell counts. Data analysis was performed using FlowJo vX.0.7 (Tree Star). The percentage of cytotoxicity was calculated with the following formula: ((relative count of GFP+ cells in Targets plus Effectors)−(relative count of GFP+ cells in Targets plus Effectors plus antibodies))/relative count of GFP+ cells in Targets.

Cell-based ELISA. Detection of trimeric Env at the surface of HOS (human osteosarcoma, ATCC) cells was performed by cell-based ELISA, as previously described (Veillette et al. J Vis Exp. 2014; 10.3791/51995:51995; Alsahafi et al. J Virol. 2018; 92: e01080-18). Briefly, HOS cells were seeded in T-75 flasks (3×10⁶ cells per flask) and transfected the next day with either 3.0 (1×), 7.5, 15.0, 22.5 or 45.0 μg per flask with the empty pcDNA3.1 vector or expressing the codon-optimized HIV-1_{JRFL} envelope glycoproteins with a truncation at position Gly 711 in the cytoplasmic tail (ΔCT), enhancing cell-surface expression. Cells were transfected with the standard polyethylenimine (PEI, Polyscience Inc, PA, USA) transfection method. Twenty-four hours after transfection, cells were plated in 384-wells plates (2×10⁴ cells per well) and one day later, cells were incubated in Blocking Buffer (Washing Buffer [25 mM Tris, ph 7.5, 1.8 mM CaCl₂, 1.0 mM MgCl₂, pH 7.5 and 140 mM NaCl] supplemented with 10 mg/ml non-fat dry milk and 5 mM Tris pH 8.0) for 30 minutes and then pre-incubated or not for 1 h with soluble CD4 (sCD4) (10 μg/ml) diluted in Blocking Buffer at room temperature. Cells were incubated with the anti-HIV-1 Env monoclonal antibodies (2G12, QA255.006, QA255.016, QA255.067, QA255.072, QA255.105, QA255.157, QA255.253, F240) in absence or presence of sCD4 (10 μg/ml) in blocking buffer. Cells were washed five times with Blocking Buffer and five times with Washing Buffer. A horseradish peroxidase (HRP) conjugated antibody specific for the Fc region of human IgG (Pierce) was then incubated with the samples for 45 minutes. Cells were washed again five times with Blocking Buffer and five times with Washing Buffer. All incubations were done at room temperature. 20 μl of a 1:1 mix of Western Lightning oxidizing and enhanced luminol reagents (Perkin Elmer Life Sciences, Waltham, Mass.) was added to each well. Chemiluminescence signal was acquired for 1 sec/well with the LB 941 TriStar luminometer (Berthold Technologies, Wildbad, Germany).

Results. Binding antibody multiplex assay (BAMA) determines specificity for gp41. Twelve antibodies from a clade A HIV-infected individual, QA255, that bound HIV clade A VLPs were previously described. One mAb (QA255.187) demonstrated modest neutralization activity. Three mAbs, QA255.105, QA255.157 and QA255.253, mediated ADCC and ADCVI activity; QA255.105 also neutralized HIV (Williams et al. EBioMedicine. 2015; 2:1464-1477). The remaining eight mAbs bound the VLP but did not mediate activity in neutralization or in ADCC assays using gp120 as a target. Unexpectedly, QA255.006 showed ADCVI activity when included as a negative control mAb in that assay despite the fact that it did not mediate ADCC against gp120-coated cells.

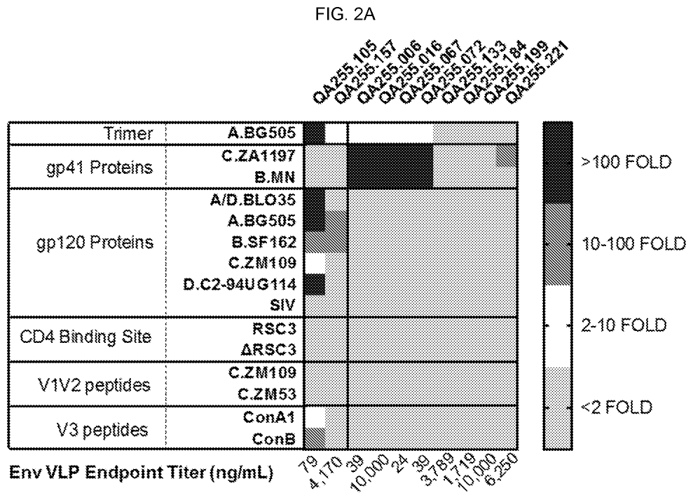

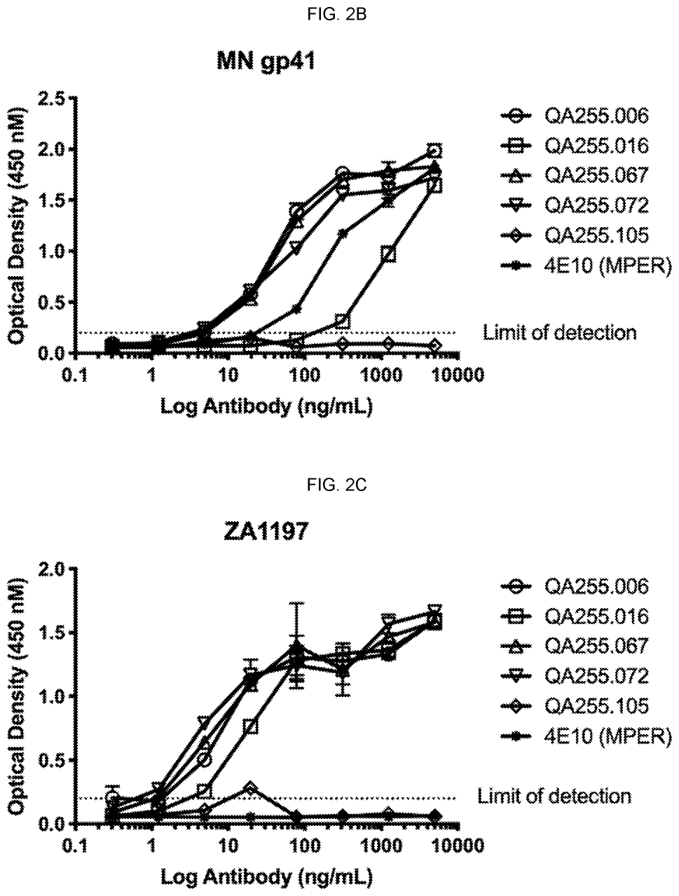

To explore the epitope specificity and function of these eight antibodies, a Binding Antibody Multiplex Assay (BAMA) that included a panel of 15 antigens was used, with two gp120-specific mAbs from QA255 serving as controls. Each antigen was individually coupled to fluorescent Luminex beads, including two gp41 proteins, five gp120 proteins representing four HIV clades and SIV, a CD4-binding site protein and negative scaffold protein, two clade C V1-V2 peptides, two V3 peptides, and BG505 SOSIP trimer (FIG. 2A). Consistent with previous findings that QA255.105 targets V3 (Williams et al. EBioMedicine. 2015; 2:1464-1477), this mAb bound to all five HIV gp120 proteins, both 175 V3 peptides and the BG505 trimer. QA255.157, which targets a CD4-induced (CD4i) epitope, bound to two of the five HIV gp120 and the BG505 SOSIP trimer. Of the eight mAbs with unknown epitopes, three did not show detectable binding to any of the proteins tested and one (QA255.221) bound to only one antigen, the gp41 ectodomain at levels just above background. Four antibodies, QA255.006, QA255.016, QA255.067 and QA255.072 bound with a range of 628- to 656-fold above background and 272- to 292-fold above background to the C.ZA.1197 gp41 ectodomain and MN gp41 proteins, respectively, suggesting that these antibodies target the gp41 portion of the HIV trimer (FIG. 2A). The very weak binding of these mAbs to the BG505 SOSIP is consistent with prior studies suggesting gp41 epitopes are largely occluded on this soluble form of the trimer (Sanders et al. PLoS Pathog. 2013; 9:e1003618).

Specificity for gp41 was confirmed by ELISA. QA255.006, QA255.067 and QA255.072 all bound to MN gp41 protein at similar levels (endpoint titer of 4.9 ng/mL), while QA255.016 displayed a less potent endpoint titer of 312.5 ng/mL. MPER-specific mAb 4E10 demonstrated intermediate binding with an endpoint titer of 78.1 ng/mL (FIG. 2B). All four antibodies demonstrated comparable binding against C.ZA.1197 ectodomain protein (endpoint titer of 4.9 ng/mL) while the MPER-specific mAb 4E10 was unable to bind the ectodomain protein at any concentration tested, consistent with the absence of MPER in this peptide (FIG. 2C). The V3-specific antibody QA255.105 did not demonstrate binding against either of the proteins at any concentration tested (FIGS. 2B, 2C).

gp41-specific antibodies demonstrate ADCC 198 activity in the RF-ADCC assay. The four gp41-specific antibodies were tested to determine whether any could mediate ADCC activity in the RF-ADCC assay (Gómez-Román et al. J Immunol Methods. 2006; 308:53-67), which has shown an association with improved HIV outcomes (Mabuka et al. PLoS Pathog. 2012; 8:e1002739; Milligan et al. Cell Host Microbe. 2015; 17:500-506). Historically, this assay has used target cells coated with gp120 protein. Given that the four QA255 mAbs targeted gp41, target cells were instead coated with the gp41 proteins used in the initial ELISA assays as well as a clade A gp140 protein, which included both gp120 and the extracellular portion of gp41.

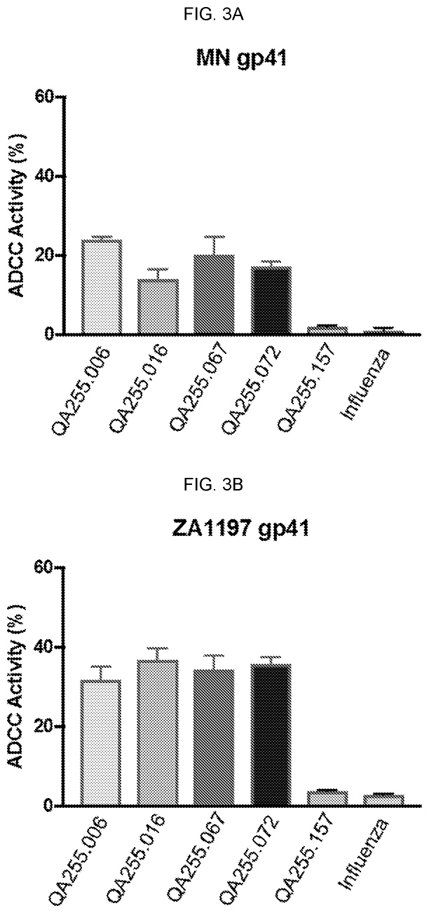

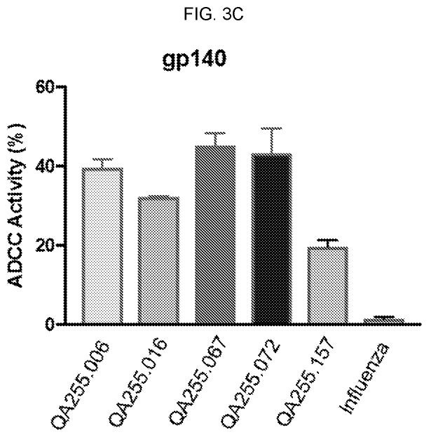

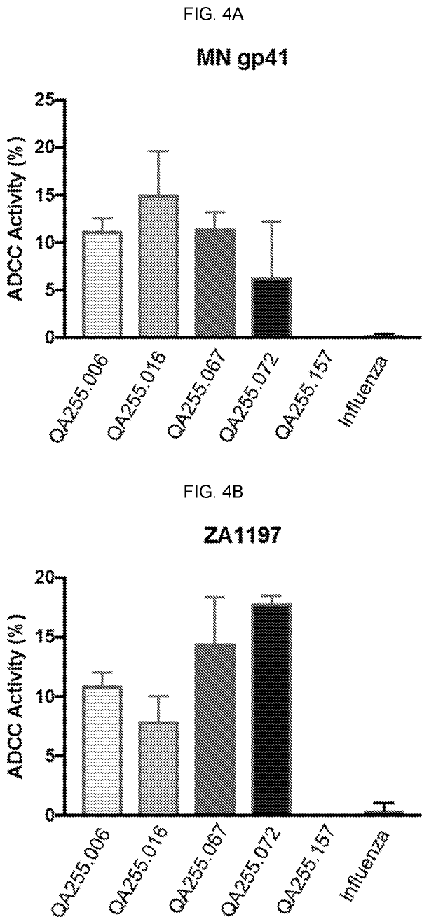

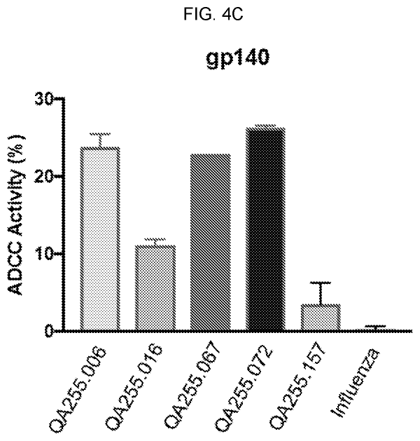

All four gp41-specific mAbs mediated robust activity against cells coated with gp140 and gp41. The four QA255 gp41-specific mAbs demonstrated between 14%-24% activity against MN gp41 and 32%-37% activity against C.ZA.1197 gp41 (FIGS. 3A, 3B). The percent ADCC activity for these four mAbs ranged from 32%-45% for cells coated with gp140, levels which were slightly higher than gp120-specific control mAb QA255.157. When tested against either of the gp41 proteins, neither QA255.157 nor an influenza-specific mAb, Fi6_v3, mediated measurable activity (FIGS. 3A-3C), as expected. Similar results were observed with PBMCs from a second donor, although the magnitude of the activity was lower (FIGS. 4A-4C).

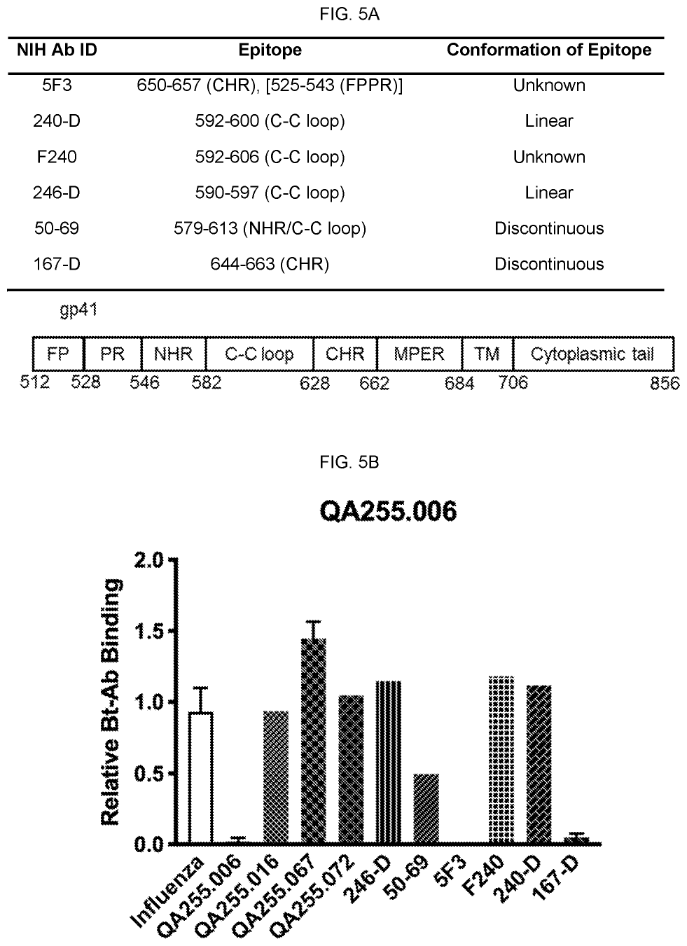

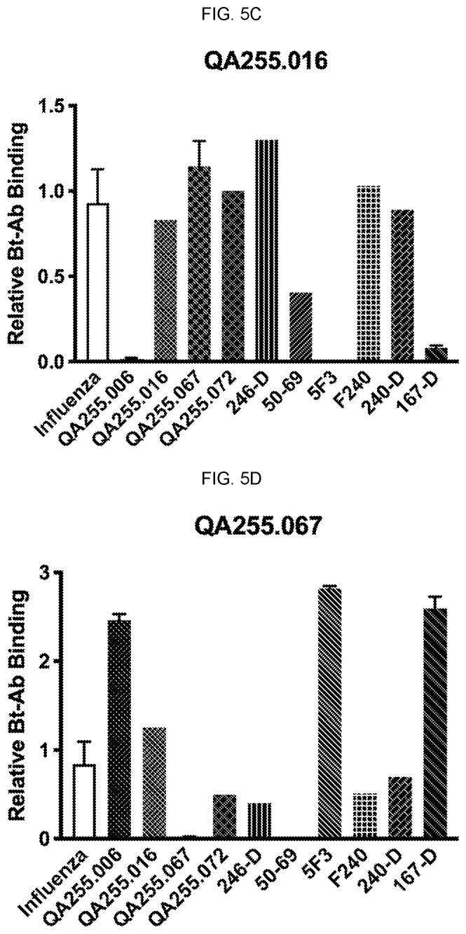

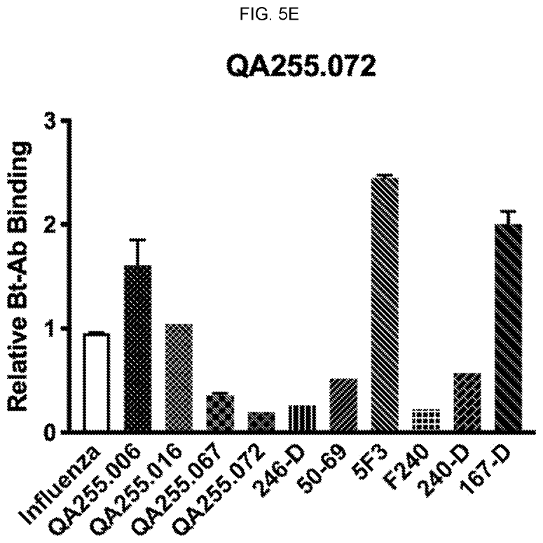

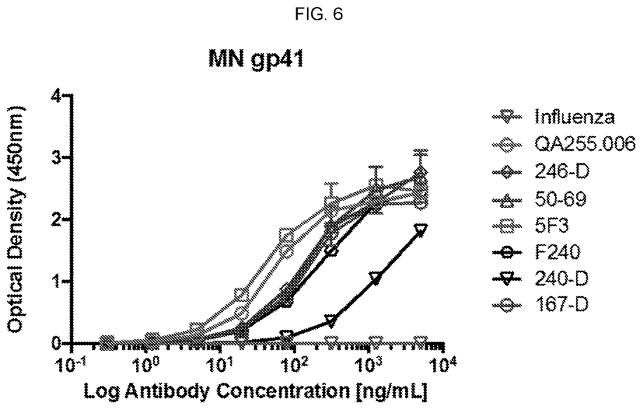

Competition ELISAs define epitope specificity for gp41-specific QA255 Abs. To begin mapping the epitope within the gp41 protein, biotinylated variants of each of the four antibodies were tested in competition with a panel of well-characterized gp41-specific antibodies that target distinct nucleotide residues spanning the ectodomain of the gp41 protein (FIG. 5A). Because all four QA255 mAbs bound with comparable efficiency to both the full gp41 protein and the C.ZA.1197 ectodomain variant of gp41 (FIGS. 2B, 2C), this suggested MPER was not the epitope target and MPER-targeting antibodies were not included in the competition ELISA. Endpoint ELISAs were performed to confirm binding for the selected six competitor mAbs against the MN gp41 protein. Five of the six mAbs bound with comparable endpoint titers between 4.9-19.5 ng/mL, while mAb 240-D demonstrated a higher endpoint titer of 78.1 ng/mL (FIG. 6).

QA255.006. The non-biotinylated version of QA255.006 reduced MN gp41 binding by the autologous biotinylated (BT) variant by 98%, however binding was not affected by the pre-incubation of the other three QA255 gp41-specific mAbs or the Influenza-specific mAb, Fi6_v3. Two mAbs, 167-D, which targets a series of discontinuous residues within CHR (Xu et al. J Virol. 1991; 65:4832-4838) and 5F3, which targets the CHR and also has been suggested to interact with the fusion proximal peptide region (FPPR) (Buchacher et al. AIDS Res Hum Retroviruses. 1994; 10:359-369; Fiebig et al. AIDS. 2009; 23:887-895), completely inhibited binding of BT-QA255.006 (95%-100%, respectively) (FIG. 5B). MAb 50-69, which targets a discontinuous epitope mapped to the NHR and the C-C loop (Xu et al. J Virol. 1991; 65:4832-4838), reduced binding of BT-QA255.006 by 51%. Overall, these patterns suggest that QA255.006 targets a discontinuous epitope that includes the CHR and possibly the FPPR and/or portions of the NHR.

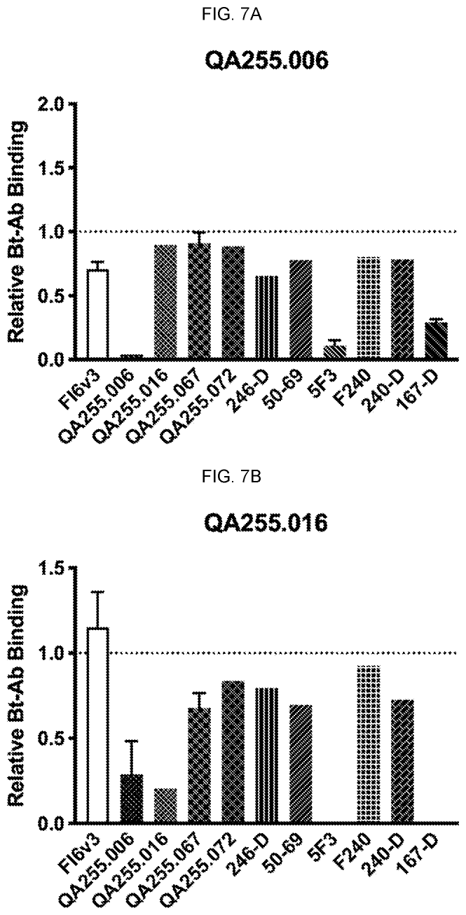

QA255.016. QA255.016 only demonstrated a modest (17%) inhibition of the biotinylated, autologous variant, whereas QA255.006 reduced BT-QA255.016 binding by 99%. Similar to the QA255.006 results, both mAbs 5F3 and 167-D strongly inhibited Bt-QA255.016 binding and mAb 50-69 partially inhibited binding by 60% (FIG. 5C). Neither QA255.067, QA255.072 nor Fi6_v3 inhibited BT-QA255.016. Thus, QA255.016 and QA255.006 appear to target a similar epitope despite originating from independent B cell progenitors (Table 1). Consistent with differences observed in ELISA endpoint titer, QA255.016 showed a more limited ability to compete in this assay as compared to QA255.006 (FIG. 2B). These data were consistent with experiments conducted using ZA.1197 ectodomain protein in place of MN gp41, with one relevant difference. When MN gp41 protein was replaced with ZA.1197 ectodomain, both QA255.006 and QA255.016 partially inhibited binding of BT-QA255.016 (FIGS. 7A, 7B).

TABLE 1
Sequence characteristics of QA255 Abs

| | V_{H} | V_{H} mut freq | J_{H} | D_{H} | V_{L} | V_{L} mut freq | J_{L} |
|---|---|---|---|---|---|---|---|
| | gene | (nt, %) | gene | gene | gene | (nt, %) | gene |
| QA255.006 | V3-23 | 6.6% | J4 | D2-8 | LV2-11 | 5.6% | J3 |
| QA255.016 | V4-34 | 11.9% | J1 | D2-15 | LV1-51 | 8.8% | J3 |
| QA255.067 | V1-69 | 10.8% | J6 | D5-18 | LV2-11 | 3.5% | J3 |
| QA255.072 | V1-69 | 13.2% | J3 | D3-22 | KV1-27 | 9.0% | J1 |

QA255.067. QA255.067 completely inhibited binding of the biotinylated, autologous variant and reduced QA255.072 binding by 51%. Pre-incubation with mAb 50-69 completely eliminated BT-QA255.067 binding, while mAbs 246-D, F240 and to a lesser extent 240-D, which all target different residues along the C-C′ loop with either linear or conformational specificity (Xu et al. J Virol. 1991; 65:4832-4838; Cavacini et al. AIDS Res Hum Retroviruses. 1998; 14:1271-1280) inhibited binding by 60%, 49% and 31%, respectively. Interestingly, pre-incubation with QA255.006, or with 5F3 or 167-D, mAbs previously shown to inhibit QA255.006 binding, increased binding over background levels measured in the absence of a competitor, suggesting that pre-incubation with these mAbs may enhance subsequent binding of QA255.067 (FIG. 5D).

QA255.072. Pre-incubation of the MN gp41 protein with either autologous QA255.072 or QA255.067 reduced BT-QA255.072 binding by comparable amounts (81% and 65%, respectively), thus suggesting that QA255.067 and QA255.072 target similar epitopes, despite also originating from independent B cell lineages (Table 1). Further, comparison between QA255.067 and QA255.072 inhibition profiles resulted in a strikingly similar pattern. As expected, pre-incubation with QA255.016 or Fi6_v3 did not inhibit QA255.072 binding, while mAbs 246-D, F240 and 240-D all reduced BT-QA255.072 binding to a degree comparable to QA255.067. The greatest deviation between the QA255.067 and QA255.072 binding properties was observed in competition with mAb 50-69, which completely eliminated QA255.067 binding, but reduced QA255.072 binding by only 49%. MAbs 5F3, 167-D and to a lesser extent, QA255.006 all appeared to exacerbate binding activity between 1.6 and 2.5-fold, consistent with observations made with QA255.067 (FIG. 5E).

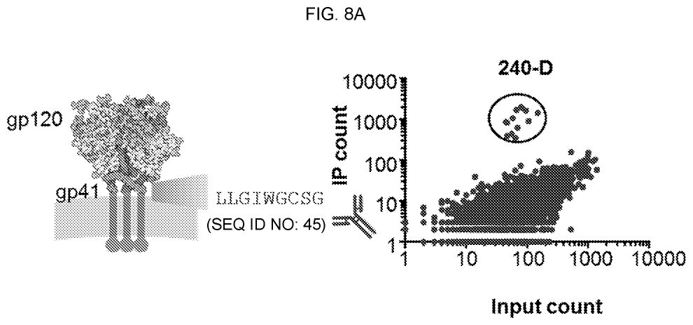

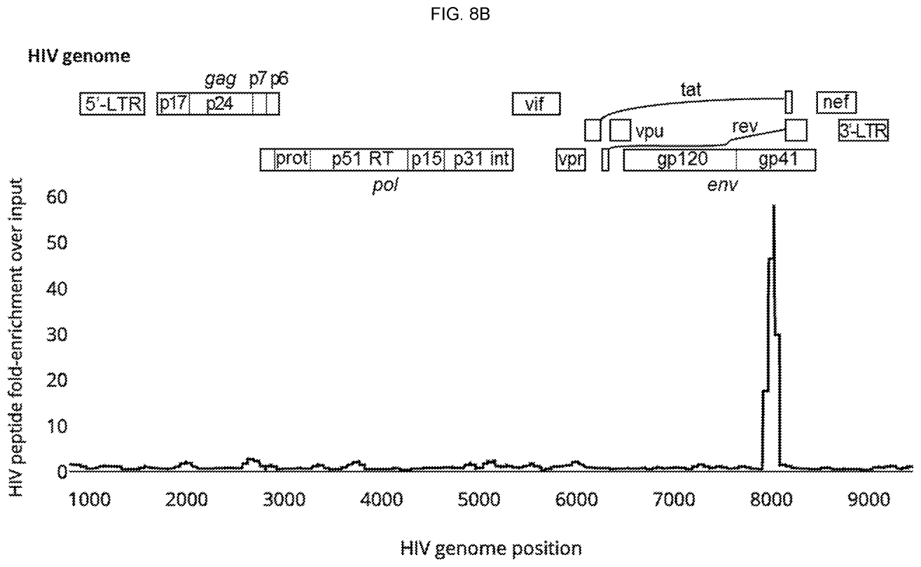

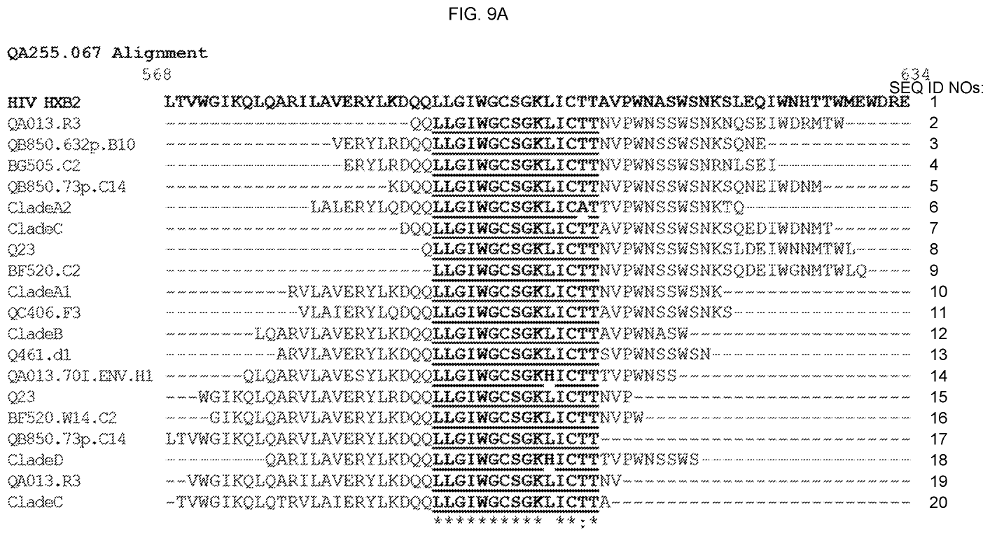

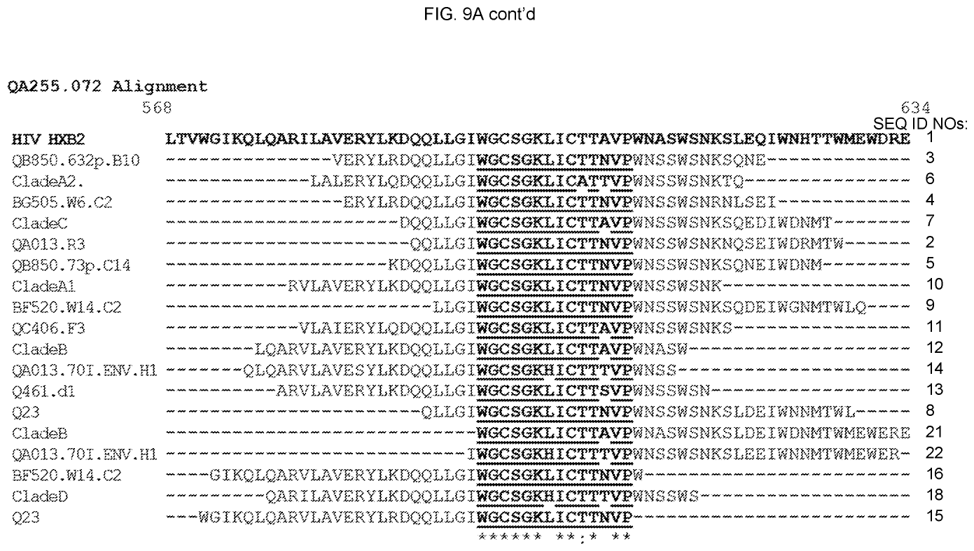

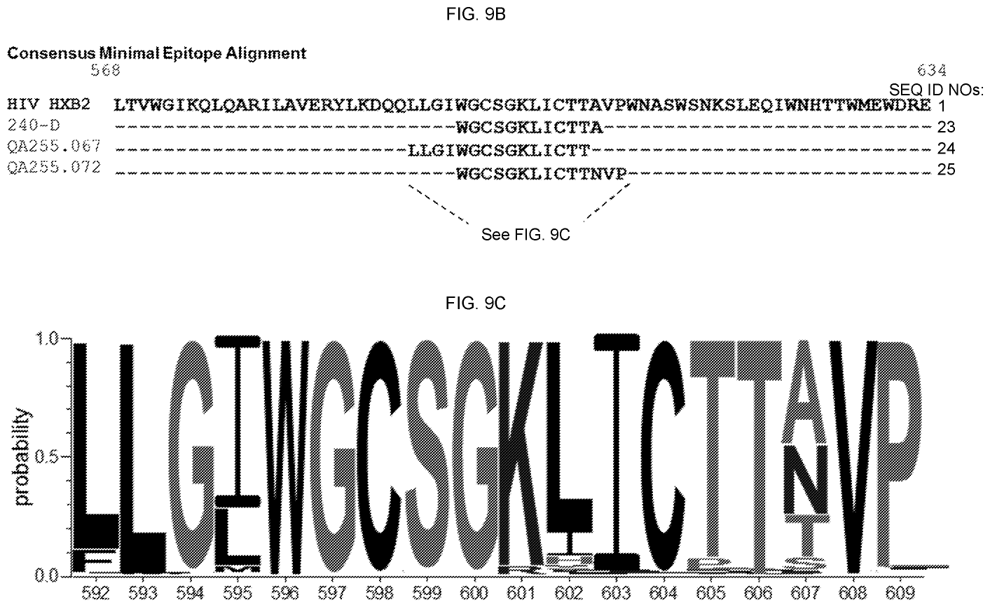

Phage peptide display identifies specific residues important for QA255.067 and QA255.072 binding. In order to more precisely map the epitopes of these mAbs, a phage immunoprecipitation sequencing approach was designed (Xu et al. Viral immunology. Science. 2015; 348:aaa0698), and peptides in the phage library that bound to the QA255 gp41-specific mAbs were determined (FIGS. 8A, 8B). A previously defined gp41-specific mAb, 240-D, was tested for comparison (Xu et al. J Virol. 1991; 65:4832-4838). The library includes sequences spanning multiple HIV Env sequences, including consensus sequences for clades A, B, C, and D and specific sequences circulating in Kenya. MAb 240-D as well as QA255.067 and QA255.072 all showed enrichment of gp41 peptides from the phage library that encoded sequences from the C-C′ loop and surrounding region, consistent with the predictions from the competition experiments. Sequences that were enriched by binding to mAb QA255.067 shared a common core sequence from 592 to 606 (based on HXB2 numbering), suggesting these amino acids are key parts of the epitope for this mAb (FIG. 9A). QA255.072 binding enriched for an overlapping but distinct peptide region that had a common core sequence of amino acids 596 to 609 (FIG. 9A). The peptides that were enriched by mAb 240-D were also similar but distinct from the QA255 mAbs and encompassed amino acids 596 to 605 (FIG. 9B), which is consistent with the known epitope originally defined by linear peptide ELISA as including 579 to 604 (Xu et al. J Virol. 1991; 65:4832-4838; Mitchell et al. Science. AIDS. 1998; 12:147-156). All HIV strains present in the phage library were represented amongst the significant hits for 240-D, QA255.067, and QA255.072. No non-Env peptides were present in the top 99th percentile of enriched peptides from 240-D, QA255.067, or QA255.072 when ranked by −log 10 p-value. MAbs QA255.006 and QA255.016 did not enrich for any peptides present in the phage library.

FIG. 9C shows a logo plot of circulating HIV sequences in the region of gp41 targeted by these mAbs indicating that the epitope target is highly conserved. In the case of QA255.067, the epitope appears to exclude the most variable amino acid in this region at position 607, although it includes the variable position 595. By contrast, QA255.072 excludes the variable position at 595 but includes the variable 607 amino acid. Interestingly, the results from phage display suggest that the QA255.072 mAb tolerates variability at position 607 as peptides with a variety of amino acids at that position are enriched. Overall, these data suggest that the QA255 gp41-specific mAbs should recognize diverse strains of HIV from different clades.

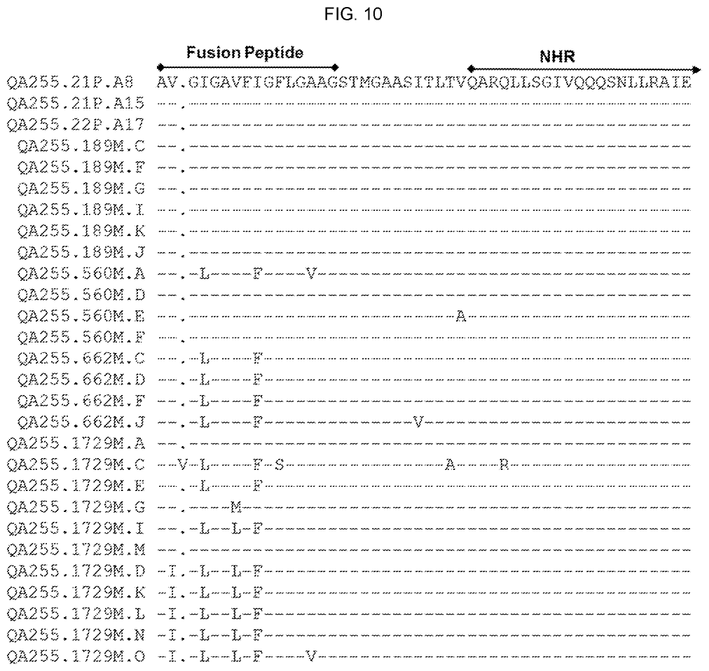

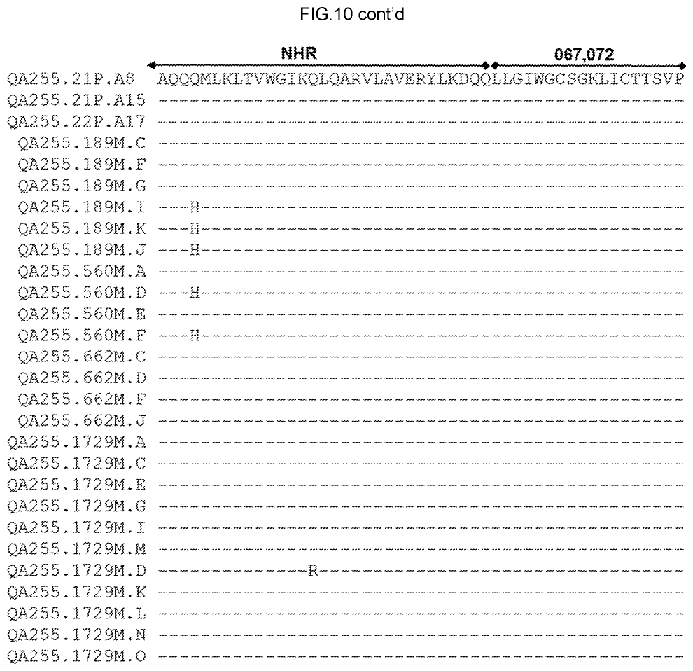

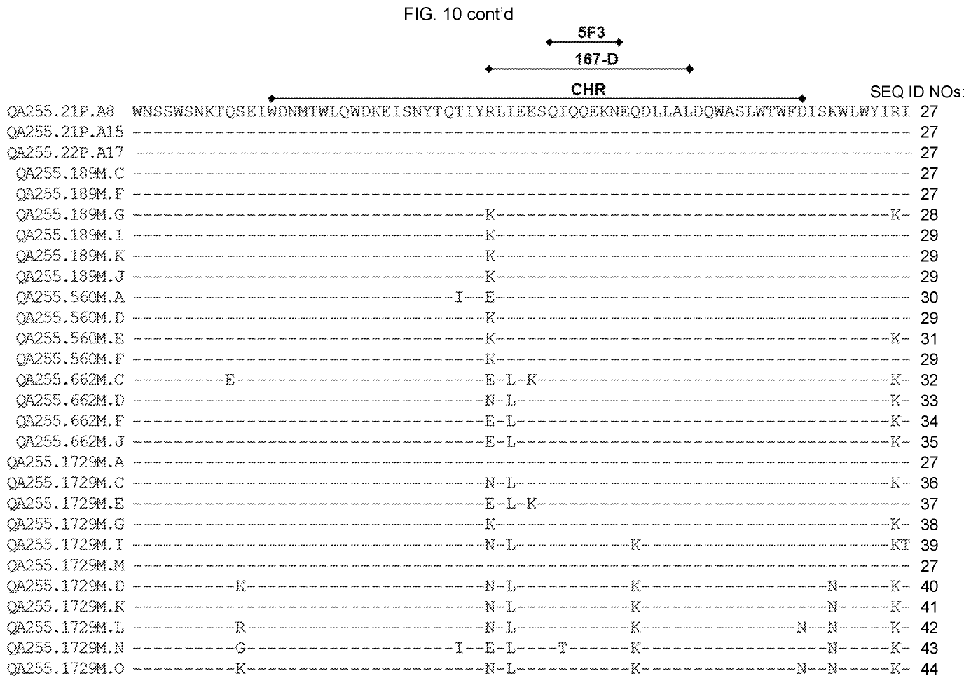

Interestingly, longitudinal sequences from QA255, starting at 21 days post-infection, show no variation within the C-C′ loop epitope of QA255.067 and QA255.072 over time (FIG. 10), perhaps reflecting the highly conserved nature of this domain. The epitopes for the mAbs QA255.006 and QA255.016 were defined only based on competition experiments with other mAbs. When the epitope of these competing mAbs (5F3 and 167-D), which are focused on the CHR and potentially the fusion peptide (Buchacher et al. AIDS Res Hum Retroviruses. 1994; 10:359-369; Xu et al. J Virol. 1991; 65:4832-4838; Fiebig et al. AIDS. 2009; 23:887-895), was examined, some evidence of variation in those regions was seen (FIG. 10). However, because the QA255.006 and QA255.016 epitopes have not been finely mapped, it is not certain that these variations are included in the actual epitope and represent escape variants.

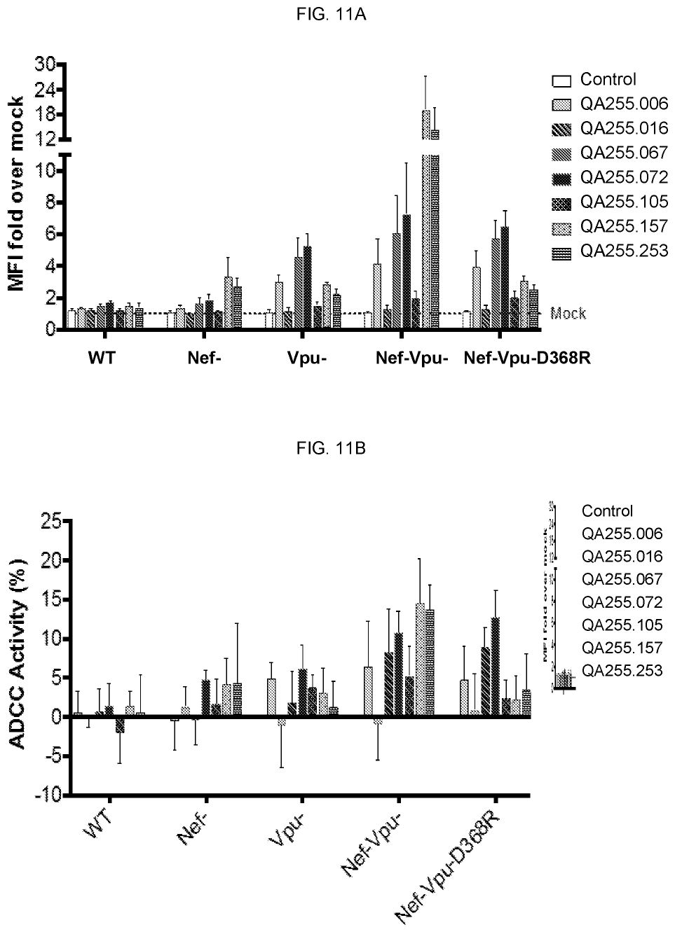

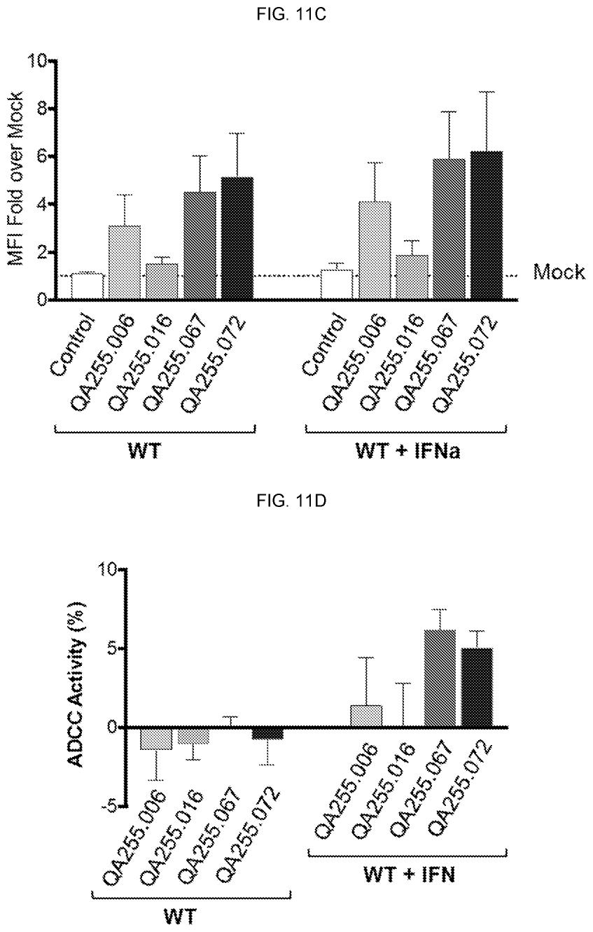

QA255.006 and QA255.016 mediate ADCC activity against a post-fusion gp41 stump mimetic. Following interaction of gp120 with CD4 and CCR5 on the surface of target cells, the gp120-gp41 complex undergoes a series of conformational rearrangements, including initial formation of a pre-hairpin fusion intermediate for virus-cell fusion followed by rearrangement into a post-fusion stable six-helix bundle. gp41 mAbs mediate killing of infected cells. All four Cluster I and Cluster II mAbs were tested for ADCC activity against cells infected with HIV-1, including viruses defective in nef and/or vpu, which leads to increased CD4 on the cell surface (Alvarez et al. J Virol. 2014; 88:6031-6046; Arias et al. Proc Natl Acad Sci USA. 2014; 111:6425-6430; Veillette et al. J Virol. 2014; 88:2633-2644), enhanced exposure of CD4i epitopes (Veillette et al. J Virol. 2014; 88:2633-2644; Alsahafi et al. J Virol. 2015; 90:2993-3002; Veillette et al. J Virol. 2015; 89:545-551) and increased Env density due to increased BST-2/Tetherin expression (Veillette et al. J Virol. 2014; 88:2633-2644; Richard et al. Trends Microbiol. 2018; 26:253-265). A third virus with defective nef and vpu genes containing a mutation in the CD4-binding site (D368R) was tested to determine whether the mAbs were dependent on conformational changes induced by CD4 interaction (Veillette et al. J Virol. 2014; 88:2633-2644; Veillette et al. J Virol. 2015; 89:545-551; Ding et al. J Virol. 2016; 90:2127-2134). The mAbs were tested with this virus panel for binding to the infected cells and ADCC activity, including with gp120-specific mAbs as controls. The gp120-specific mAbs, QA255.157 and QA255.253 (Williams et al. EBioMedicine. 2015; 2:1464-1477), showed the highest level of binding to infected cells and corresponding high ADCC activity against cells infected with virus containing both defective nef and vpu genes (FIGS. 11A, 11B). As expected, this activity was impaired in cells infected with the D368R construct that eliminated CD4-Env binding and therefore exposure of CD4i epitopes (Veillette et al. J Virol. 2015; 89:545-551; Ding et al. J Virol. 2016; 90:2127-2134).

When the infected cell panel was tested against the four gp41 mAbs, all showed very little binding to cells infected with the wild type virus and this translated into no ADCC activity against these cells. QA255.006, QA255.067 and QA255.072 showed increased binding and ADCC activity against cells infected with the Vpu-deficient virus and the Vpu- and Nef-deficient viruses while QA255.016 showed barely detectable binding or ADCC activity against cells infected with all the viruses tested. This is consistent with Env accumulation at the surface of cells infected with Vpu-deficient viruses, as BST-2/Tetherin can mediate retention of viral particles (Alvarez et al. J Virol. 2014; 88:6031-6046; Arias et al. Proc Natl Acad Sci USA. 2014; 111:6425-6430) on the cell surface. Accordingly, Interferon alpha (IFNα) treatment, which also enhances BST-2 retention of viral particles at the cell surface (Arias et al. Proc Natl Acad Sci USA. 2014; 111:6425-6430; Richard et al. J Virol. 2017; 91: pii: e00219-17), increased both recognition (FIG. 11C) and ADCC susceptibility (FIG. 11D) of cells infected with wild-type viruses. Finally, no increase above wild type levels for cells infected with the Nef-deficient virus was observed. For all of these mAbs, the presence of a mutation in the CD4 binding site (D368R) did not impact binding or ADCC activity, suggesting the epitopes recognized by these Abs are not dependent on structural changes that occur upon Env-membrane-bound CD4 interaction.

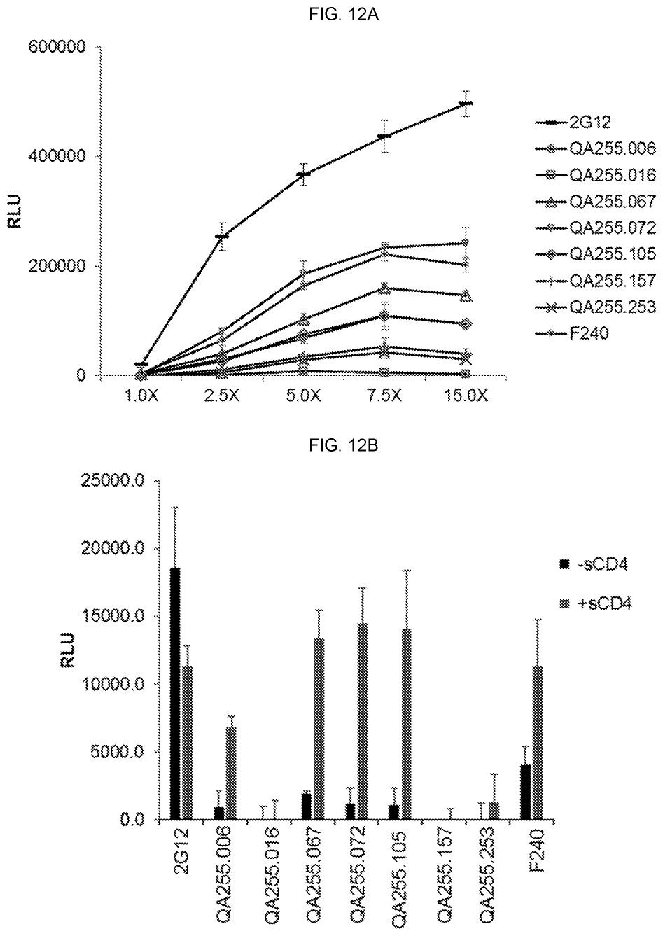

Because the conditions that allowed detection of ADCC activity in the infected cell assay were when BST-2 levels promoted capture of viral particles, it could not be determined if the gp41 mAbs are capable of binding to gp41 on the cell surface or their binding reflects interaction with trapped viral particles, which would be consistent with the fact that they were isolated using viral particles as a bait (Williams et al. EBioMedicine. 2015; 2:1464-1477). To address this, the gp41 mAbs were tested in a cell-based ELISA assay where only Env is expressed at the cell surface (Veillette et al. J Vis Exp. 2014; 10.3791/51995:51995). QA255.006, QA255.067 and QA255.072 were able to bind Env at the cell surface, with higher binding detected at higher Env levels (as detected by 2G12, FIG. 12A). Consistent with poor recognition of infected cells by QA255.016 (FIGS. 11A-11D), no binding for this Ab was observed in this system (FIG. 12A). Thus, these data indicate that these gp41 mAbs do not require viral particles to interact with Env. Consistent with their ability to recognize a gp41 stump mimetic (FIGS. 3A-3C), it was observed that sCD4-induced shedding as indicated by decreased 2G12 levels upon sCD4 addition, dramatically increased the ability of these mAbs to recognize Env (FIG. 12B) further supporting the possibility that these mAbs recognize gp41 stumps. In addition, the same pattern is also seen for the anti-gp41 F240 mAb, which has also been suggested to recognize gp41 stumps (Gohain et al. Sci Rep. 2016; 6: 36685). How can this be reconciled with the observation that these mAbs do not more efficiently recognize cells infected with a virus deleted in Nef, which have higher levels of CD4 compared to cells infected with Nef containing virus (Veillette et al. J Virol. 2014; 88:2633-2644; Alsahafi et al. J Virol. 2015; 90:2993-3002)? A potential explanation is that in cells infected with Nef-virus, CD4 interacts with Env in cis, thus occluding the access to the epitope, which is not the case when the Env is opened using sCD4. Supporting this, 8ANC195 does not efficiently recognize cells infected with Nef-virus (Ding et al. J Virol. 2016; 90:2127-2134) despite the fact that the structure of this mAb was obtained using a gp120 core stabilized with sCD4 (Scharf et al. Cell Rep. 2014; 7:785-795).

There has been renewed interest in antibodies that mediate ADCC based on findings that ADCC antibody activity was associated with protection in the RV144 vaccine clinical trial (Haynes et al. N Engl J Med. 2012; 366:1275-1286) and in the setting of mother-to-child transmission (Mabuka et al. PLoS Pathog. 2012; 8:e1002739; Milligan et al. Cell Host Microbe. 2015; 17:500-506). In addition, non-neutralizing ADCC antibodies have been associated with protection and delayed disease in NHP vaccine models and reduced viremia when passively infused prior to infection of NHP (Lewis et al. Immunol Rev. 2017; 275:271-284). This example describes four new gp41-specific ADCC mAbs that arose from four independent B cell lineages in one clade A infected individual. Two of these mAbs also recognize gp41 stumps and mediate ADCC against cells coated with stump mimetics. Importantly, these mAbs can mediate cell killing in multiple assays, including killing of productively infected T cells, the major source of virus in HIV infection. Notably, they mediate killing in infected cells exposed to IFN, a condition that is likely to be relevant to HIV infection in vivo.

The epitopes of these gp41 mAbs were mapped using both competition experiments and phage peptide display. Immunoprecipitation of a library of phage has the advantage of being able to interrogate a large number of peptides in a single well using deep sequencing to identify the specific peptides within the library that bind the mAbs. The present experiments showed that QA255.067 and QA255.072 target the immunodominant C-C′ loop, which suggests they target cluster I. The phage display method allowed the defining of a minimal epitope based on overlap in the sequences that bound these mAbs. These results suggest that there are subtle differences in the epitopes of these mAbs and also in these epitopes compared to a previously defined cluster I mAb, 240-D. Interestingly, the minimal epitope of QA255.067 excludes a variable residue at position 607, whereas this residue is included in the minimal epitopes of QA255.072 and variation at this residue appears to be tolerated by mAb QA255.072. Interestingly, longitudinal viruses cloned from QA255 over a more than four-year time period after infection demonstrated no variation in these residues, thus suggesting that ADCC antibody pressure is not sufficient to drive escape in this highly conserved region of gp41. The epitope of a previously described mAb, 240-D, was also mapped to amino acids 596-605, which refined the epitope compared to the original 240-D epitope mapping study, which indicated the epitope was between 579-604 based on peptide binding studies (Xu et al. J Virol. 1991; 65:4832-4838). The present results are also consistent with later studies examining binding to mutant forms of Env-gp160 protein, which suggested mutations at positions 596, 599 and 605 impact 240-D binding (Mitchell et al. AIDS. 1998; 12:147-156). Overall, this analysis suggests that phage display could provide a high throughput tool for epitope mapping.

While the application of phage immunoprecipitation with deep sequencing was successful for mapping the epitopes of the cluster I mAbs, it was not successful for the mAbs with more complex, discontinuous epitopes. QA255.006 and QA255.016 share some properties of cluster II mAbs in that competition studies suggest their epitope includes the CHR. But competition experiments suggest that the target of these mAbs may also be discontinuous and include the fusion peptide proximal region and/or the NHR. These mAbs appear to enhance binding of the C-C′-loop, cluster I mAbs, as do other mAbs that target the CHR, such as 5F3. Interestingly, a mAb that bound a complex epitope on HIV gp41 was isolated from a clade B infected individual using VLPs to enrich for HIV-specific B cells suggesting these types of mAbs may be readily detected using VLPs as bait (Hicar et al. Mol Immunol. 2016; 70:94-103).

The QA255 derived gp41 mAbs all demonstrated measurable ADCC activity against cells coated with gp41, including the ectodomain expressed alone as well as within the context of the gp140 protein. Importantly, they also mediated killing of infected target cells, although the activity driven by mAb QA255.016 was very low. Poor ADCC activity by QA255.016 is consistent with the competition assay observations where QA255.006 was able to displace Bt-QA255.016 but QA255.016 was unable to displace BT-QA255.006 and with preliminary Bio-layer interferometry results that suggested weaker binding as compared to QA255.006 using gp41 protein ZA.1197 (data not shown). Activity was more readily detected with viruses lacking Vpu, presumably because the cells infected with vpu-deleted viruses have higher cell surface Env expression due to trapped viral particles (Alvarez et al. J Virol. 2014; 88:6031-6046; Arias et al. Proc Natl Acad Sci USA. 2014; 111:6425-6430; Veillette et al. J Virol. 2014; 88:2633-2644; Richard et al. J Virol. 2017; 91: pii: e00219-17; Neil et al. Nature. 2008; 451:425-430; Van Damme et al. Cell Host Microbe. 2008; 3:245-252). Accordingly, stimulation with IFNα, known to induce retention of viral particles at the surface of infected cells (Arias et al. Proc Natl Acad Sci USA. 2014; 111:6425-6430; Richard et al. J Virol. 2017; 91: pii: e00219-17), increased recognition and ADCC activity of these Abs. The activity was not dependent on Env-CD4 interaction at the cell surface because it was not increased compared to wild type virus when a nef-deleted virus was used. CD4i epitopes are a common target of non-neutralizing gp120-specific mAbs that mediate ADCC and can result in killing of bystander cells that have shed gp120 on their surface (Richard et al. Trends Microbiol. 2018; 26:253-265; Richard et al. EBioMedicine. 2016; 12:208-218). As such, the gp41 mAbs would be predicted to have fewer off-target effects that result in this undesirable killing of HIV negative cells.

The presence of gp41 antibodies that mediate ADCC in plasma has long been appreciated (Evans et al. AIDS. 1989; 3:273-276; Koup et al. J Virol. 1989; 63:584-590). Many previous studies showed that gp41-directed antibody responses are generally common in HIV-infection, including responses to epitopes that are similar to those of the mAbs studied here (Gnann et al. J Infect Dis. 1987; 156:261-267; Xu et al. J Virol. 1991; 65:4832-4838; Gnann et al. J Virol. 1987; 61:2639-2641; Klasse et al. Proc Natl Acad Sci USA. 1988; 85:5225-5229; Wang et al. Proc Natl Acad Sci USA. 1986; 83:6159-6163). Despite the common nature of gp41 plasma antibody responses, relatively few gp41-specific mAbs that mediate ADCC have been described. Many of the previously characterized mAbs are IgG2 (Forthal et al. AIDS Res Hum Retroviruses. 1995; 11:1095-1099; Tyler et al. J Immunol. 1990; 145:3276-3282), an isotype which primarily mediates killing via macrophages and neutrophils through the FcγRIIa. IgG2 also has very low affinity for the Fc receptor most important for NK-cell mediated ADCC activity, FcRγIIIa (Vidarsson et al. Front Immunol. 2014; 5:520). The gp41 ADCC mAbs described here were encoded as IgG1, which can interact with a range of FcγRs. IgG1 is also the most abundant antibody and thus a major driver of the ADCC response. In addition to ADCC, gp41-specific mAbs have been shown to block transcytosis of virus (Shen et al. J Immunol. 2010; 184:3648-3655; Tudor et al. Mucosal Immunol. 2009; 2:412-426) and to inhibit virus infection in dendritic cells and macrophages by mechanisms that likely involve effector functions (Holl et al. J Virol. 2006; 80:6177-6181; Peressin et al. J Virol. 2011; 85:1077-1085). Moreover, gp41-specific IgA activity has been linked to resistance from infection in highly exposed seronegative individuals (Pastori et al. J Biol Regul Homeost Agents. 2000; 14:15-21). Thus overall, gp41-specific antibodies may make unique contributions to decreasing HIV transmission and pathogenesis. In this regard, the effect of IFN on ADCC activity observed here may be particularly relevant given that IFN is an early antiviral response.

Four of the twelve HIV-specific mAbs isolated from a clade A infected individual targeted gp41 and they were all derived from independent lineages, even though there were two pairs of mAbs, with each pair targeting similar epitopes. This suggests that gp41-specific mAbs that mediate ADCC may be a common response during chronic HIV infection and the antibodies isolated here will be useful as reagents for testing this hypothesis. These ADCC mAbs from 914 days post infection showed relatively low somatic hypermutation (SHM) (VH: 6.5-12.9%; VL/VK: 3.7-8.8% NT) (Table 1) compared to broadly neutralizing mAbs. Two of the four gp41-specific mAbs described here, QA255.067 and QA255.072, utilize gene IGVH1-69 (Table 1), which is common for cluster I-directed mAbs (Gorny et al. Mol Immunol. 2009; 46:917-926).

One of the challenges in eliciting a protective response against HIV, particularly for eliciting protective neutralizing antibodies, is the diversity of the Env antigen. To date, the gp41-specific mAbs identified after HIV vaccination have tended to be polyreactive and not able to mediate HIV-specific ADCC activity (Williams et al. Science. 2015; 349:aab1253). ADCC Abs tend to target conserved epitopes and show breadth (Lewis et al. Immunol Rev. 2017; 275:271-284; Williams et al. EBioMedicine. 2015; 2:1464-1477; Madhavi et al. AIDS. 2014; 28:1859-1870; Mayr et al. Sci Rep. 2017; 7:12655; McLean et al. J. Immunol. 2017; 199:816-826; Ramirez Valdez et al. Virology. 2015; 475:187-203]. In terms of breadth of the gp41 protein, in particular the ectodomain, gp41 is a particularly attractive target because it is more conserved than most gp120 regions targeted by bnAbs (Steckbeck et al. J Biol Chem. 2011; 286:27156-27166). Thus, the new ADCC Abs described here, that target conserved regions in gp41 and mediate killing of HIV-infected cells may provide insight into the features of antibodies that can mediate broad protection against HIV infection.

Example 2. A phage display approach maps linear epitopes of gp41-specific mAbs that mediate ADCC.

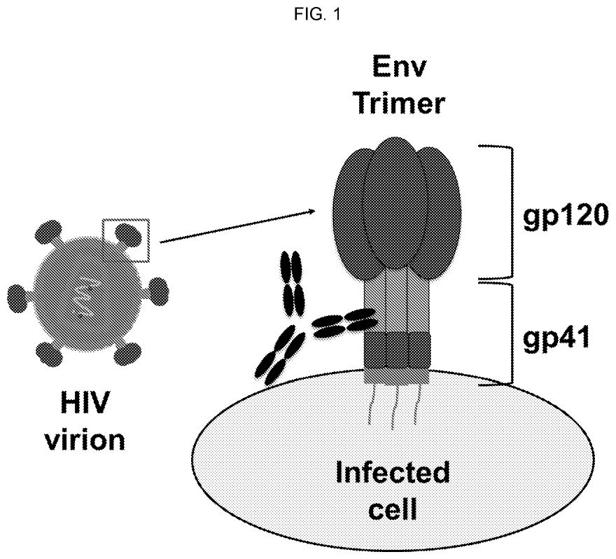

An HIV virion includes an envelope protein including glycoprotein 41 (gp41) and glycoprotein 120 (gp120) (FIG. 1). Antibodies that mediate killing of HIV-infected cells through antibody-dependent cellular cytotoxicity (ADCC) have been implicated in protection from HIV infection and disease progression. Deep mutational scanning (DMS) is a massively parallel method of interrogating the role of each amino acid in protein-protein binding interactions (Dingens et al. Cell Host and Microbe. 2017; 21(6):777-787; Dingens et al. Immunity. 2019 Jan. 29). DMS can be used to map epitopes of HIV-specific monoclonal antibodies in a method called mutational antigenic profiling; however, mutational antigenic profiling that incorporates DMS uses infectious HIV and requires high volumes of HIV for each experiment, limiting the number of experiments that are possible. This approach also requires that the antibody neutralize the virus. Currently a high-throughput, inexpensive, rapid way to map the epitope of mAbs that bind to viral proteins, irrespective of their ability to neutralize virus, is lacking. This Example describes an epitope mapping strategy applied to mAbs that bind HIV and mediate ADCC isolated from an HIV infected individual.

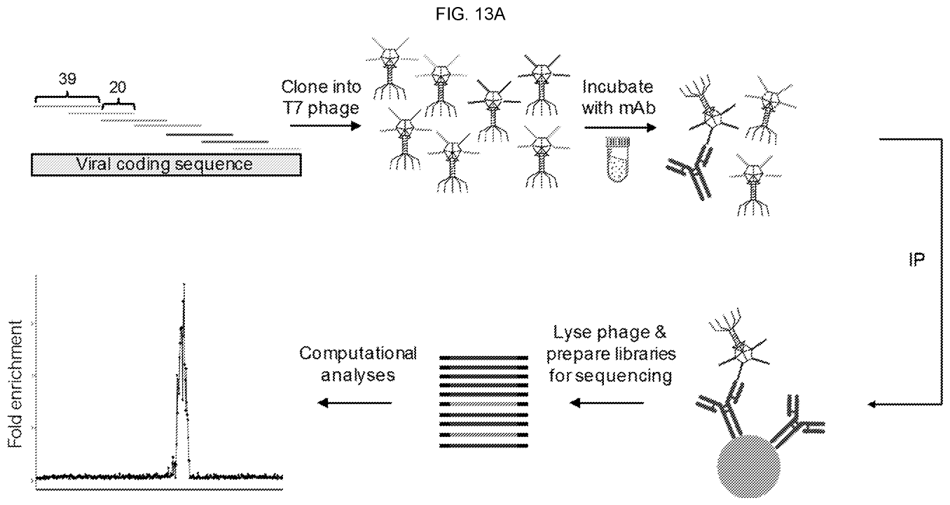

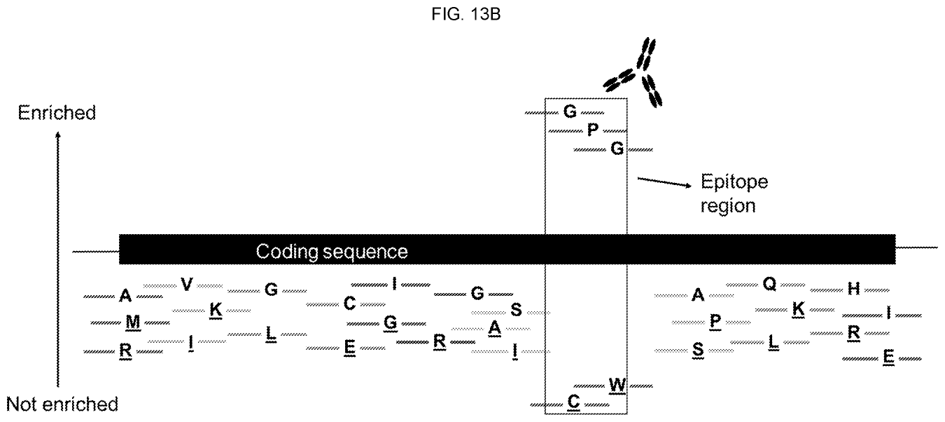

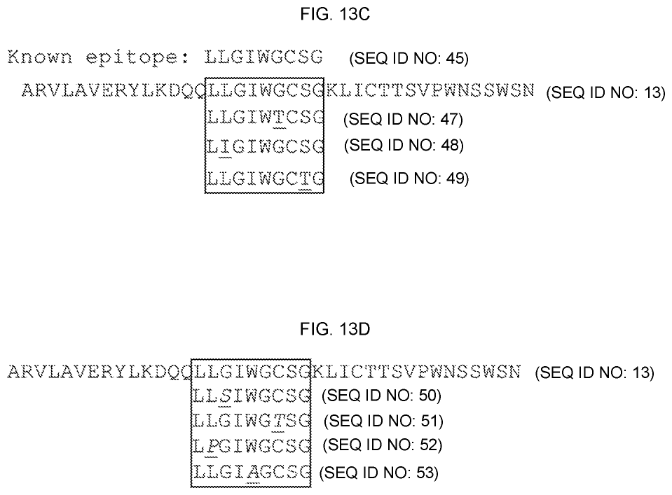

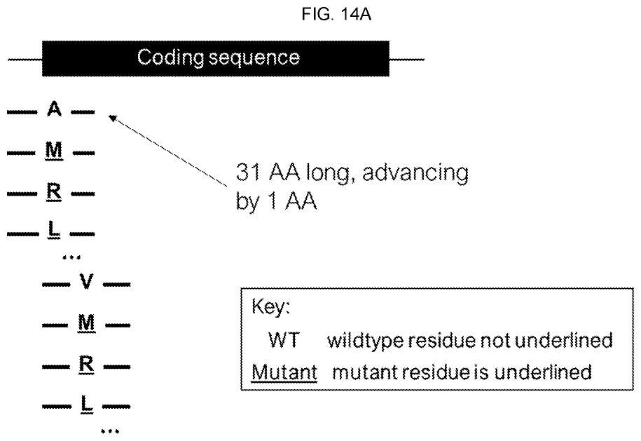

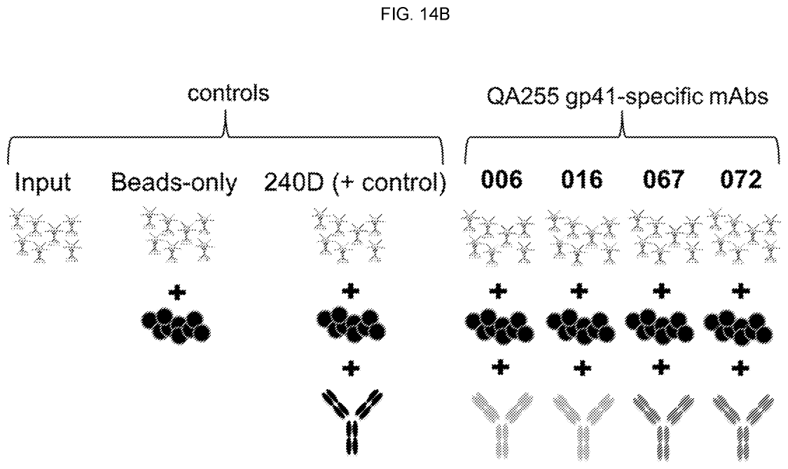

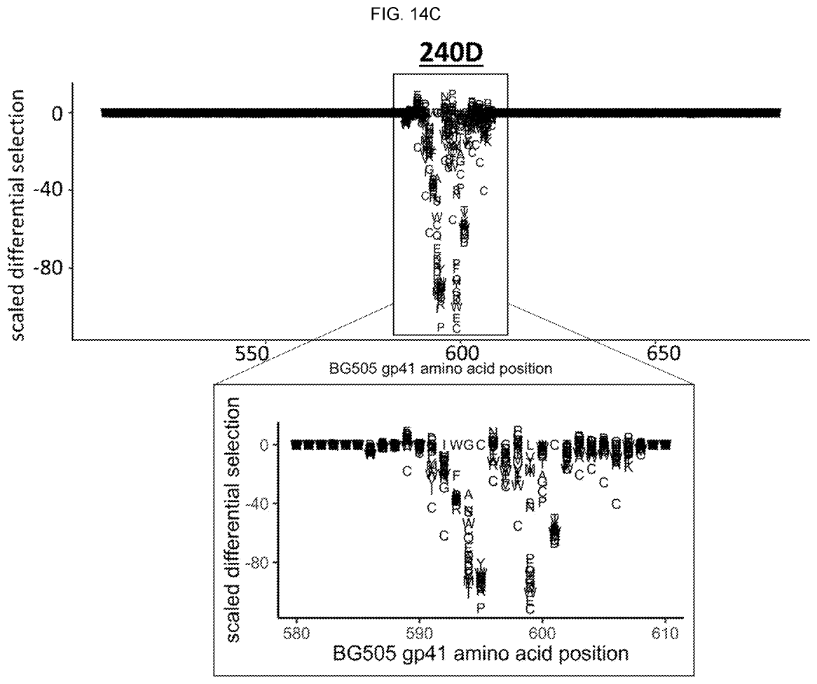

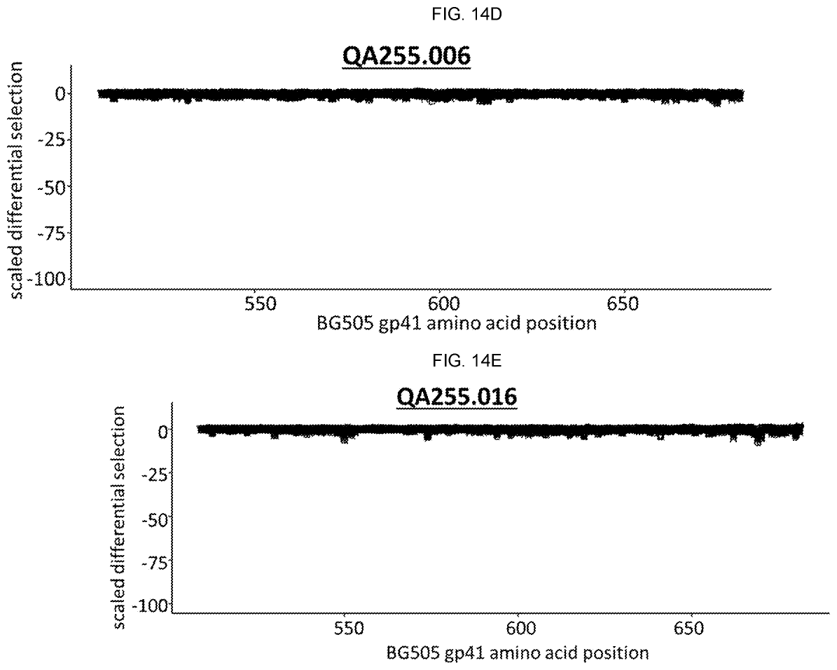

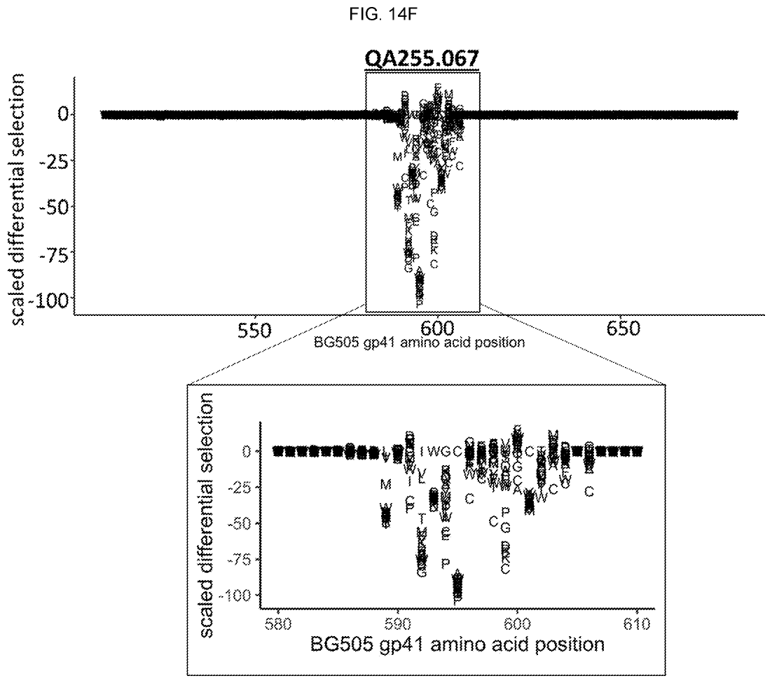

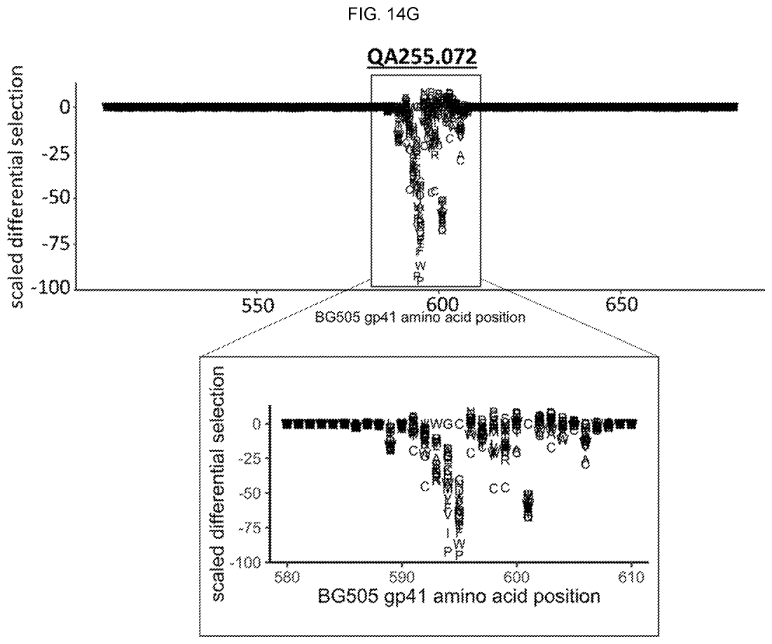

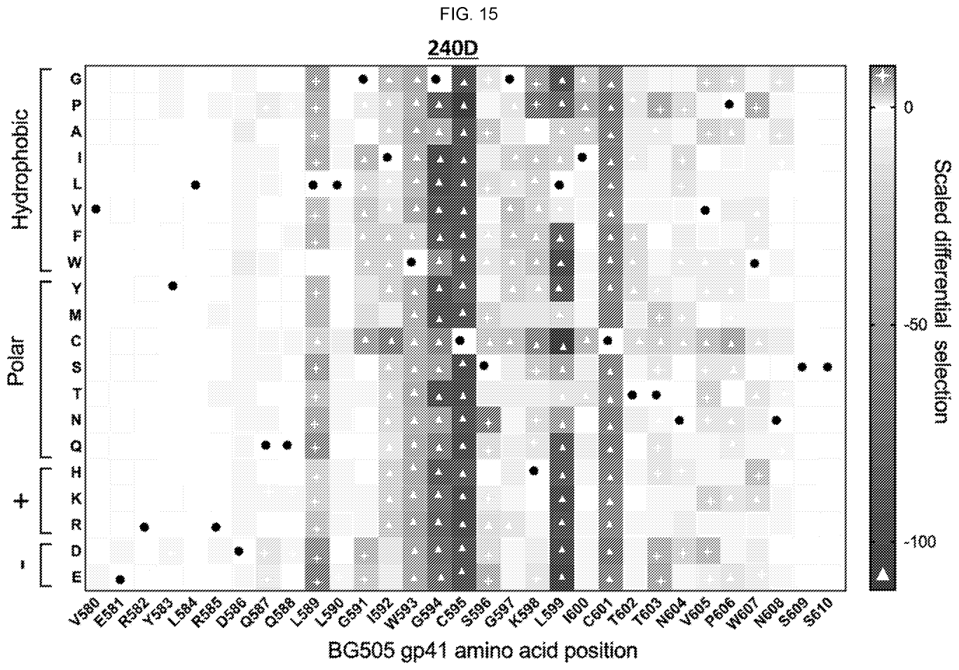

Generation of a DMS phage display library. A library of tiled peptides 31 amino acids in length was generated with either a wildtype or mutant residue at the central amino acid, across the ectodomain of gp41. The DMS phage display library contains peptides sampling every possible single-amino acid (FIG. 14A). gp41 sequences in the library included: BF520.W14.C2, BG505.W6.C2, and ZA1197. After generation of the library of sequences, the viral coding sequences were cloned into T7 phage to express the corresponding peptides (FIG. 13A). Using Phage Immunoprecipitation-Sequencing (Ph-IP-Seq), the phage were incubated with the monoclonal antibody (mAb), the antibody-bound phage were immunoprecipitated, and the phage were lysed and deep sequenced. Enriched sequences were identified through computational analyses (FIG. 13A). FIG. 14B shows a setup of a DMS/phage experiment with the identified gp41 mAbs described in Example 1 and a positive control (240D) with a defined gp41 epitope. FIG. 13B shows expected results of this experiment with enriched peptide sequences and peptide sequences not enriched. Enriched peptides spanning the epitope region (indicated by a box) have mutations that tolerate the epitope, whereas peptides spanning the epitope region that are not enriched have mutations that disrupt the epitope and allow escape. FIG. 13C shows a hypothetical example of mutations (underlined) in enriched peptide sequences that are tolerated/do not disrupt the epitope, while FIG. 13D shows representative mutations (italicized and underlined) in non-enriched peptide sequences that disrupt the epitopes and allow escape.

The positive control mAb 240D bound to peptides with the expected amino acids as defined by prior studies (FIG. 14C). The scaled differential selection values are displayed, with the WT amino acid at 0 on the y-axis and mutant amino acids either above or below WT. QA255.006 (FIG. 14D) and QA255.016 (FIG. 14E) did not significantly enrich for any peptides above background. Both QA255.067 (FIG. 14F) and QA255.072 (FIG. 14G) significantly enriched for peptides spanning the immunodominant C-C loop region of gp41, with certain mutations in this region abolishing mAb binding, indicating they disrupt a residue critical to the epitope.

Phage-DMS reveals sites of binding between gp41 peptides from HIV strain BG505 and mAb 240D. Phage-DMS results are displayed in heatmap form across amino acid positions 580-610. The wild type amino acid in BG505 is indicated with the amino acid number at the bottom of each column and these are also shown as dots in the figure. The rows show the results for amino acid residue at each of the positions, grouped by the characteristics of the amino acid. Mutations to sites resulting in a loss of binding relative to WT have a white triangle in the box and sites that result in increased binding have a white, four-point star in the box. These results are consistent with the known epitope of 240D; for example, the C at position 595 is critical to the epitope and all changes to that position decrease binding. The G at position 594 and the L at position 599 are also preferred amino acids for the 240D mAb.

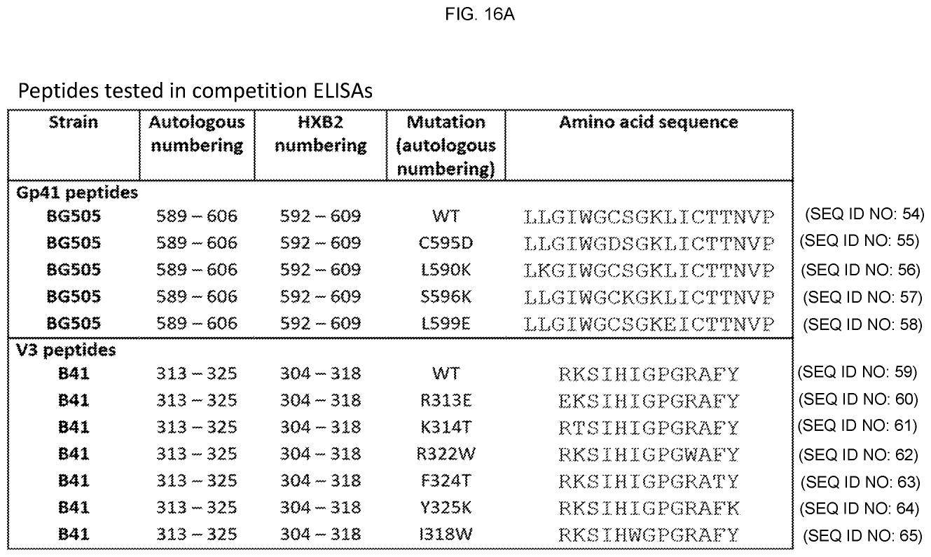

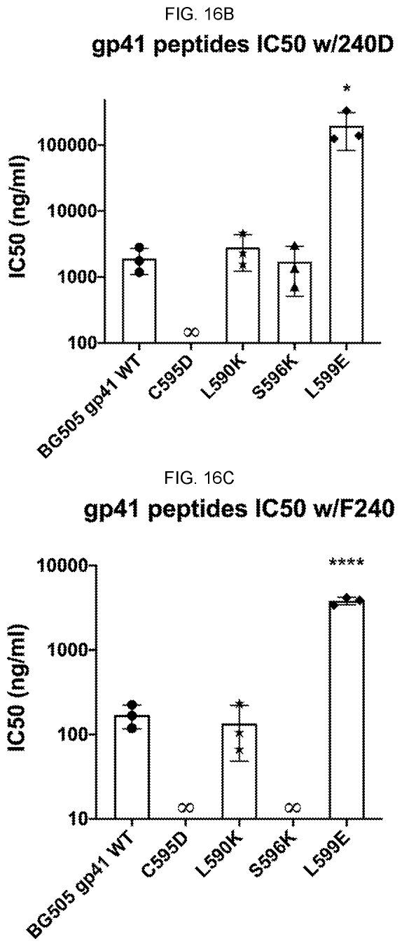

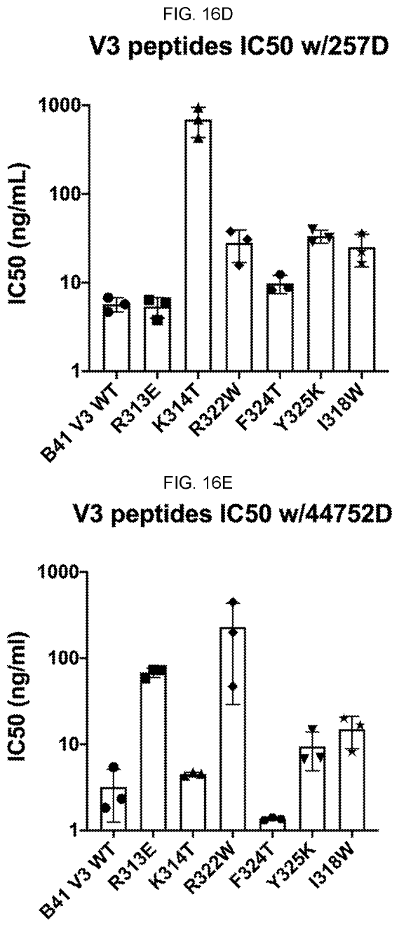

Results of antibody binding assays by an ELISA for various peptide variants that were predicted to have altered binding by Phage-DMS. Select mutant peptides predicted by Phage-DMS to either increase or decrease binding to gp41 mAbs and V3 mAbs mAbs were synthesized and are shown in FIG. 16A. The strain of the HIV in the Phage-DMS library that these variants are based on is indicated along with the amino acid positions with the protein based on standard HIV HXB2 numbering. These peptides were tested in a peptide competition ELISA: gp41 peptides were preincubated with the gp41-specific antibodies 240D (FIG. 16B) and F240 (FIG. 16C) and V3 peptides were preincubated with the V3-specific antibodies 447-52D (FIG. 16D) and 257D (FIG. 16E) before performing an ELISA. An IC50 value was calculated for each peptide to quantify the effect of each mutation on antibody binding. An IC50 that is higher than the wildtype suggest that the amino acid variant binds better to the mAb than wildtype whereas a lower IC50 indicates the amino acid variant leads to reduced binding. * indicate statistically significant differences. The results include three different experiments.

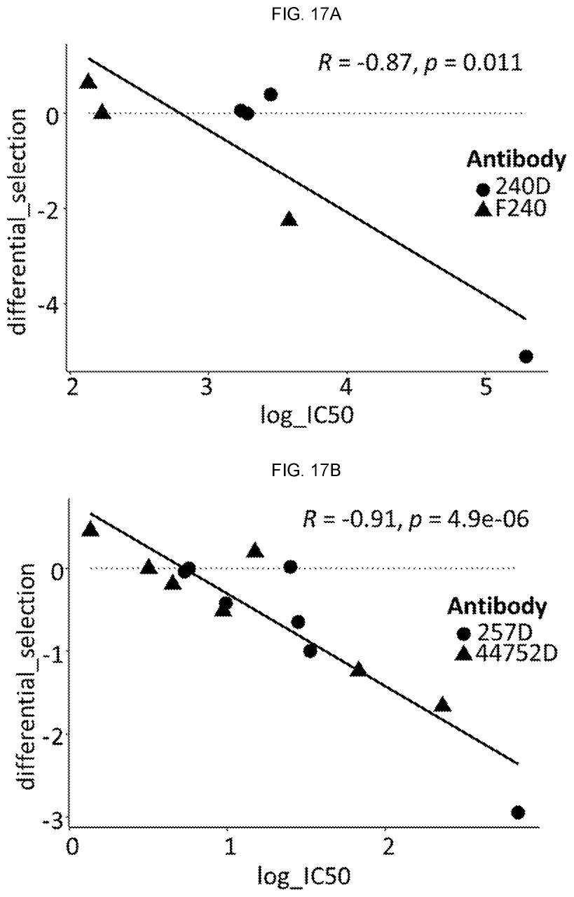

Correlation between Phage-DMS results and the competition ELISA. Scaled differential selection values as determined by Phage-DMS were correlated with the IC50 value determined by competition peptide ELISA for each mutation examined in the ELISA. Results with gp41-specific antibodies (FIG. 17A) and V3-specific antibodies (FIG. 17B) are shown. The Pearson correlation coefficient along with the p-value is displayed.

The epitope of two (QA255.067 and QA255.072) of the four gp41 mAbs described in Example 1 were finely mapped using a DMS phage display approach that revealed specific amino acids critical for binding within and just outside the C-C loop of gp41. The other two gp41 mAbs described in Example 1 (QA255.006 and QA255.016) recognize a discontinuous epitope on gp41 consisting of the FPPR and CHR, as indicated by competition ELISA experiments, and could not be further mapped using this DMS phage display approach. Importantly, mutations that were enriched or depleted in Phage-DMS, suggesting that they increase or decrease binding, respectively, showed similar results in more commonly used ELISA method to study binding. Overall, this DMS phage display method provides a rapid, high throughput way of mapping the linear epitopes of antibodies at single amino acid resolution and enables the identification of mutations that disrupt the epitope and allow escape of virus from antibody recognition. The disclosed DMS phage display method is high-throughput and cost-effective. Once the library is made, the phage can easily be regrown and the main cost would be sequencing. The phage library can also be easily regenerated. The DMS phage display approach does not require large volumes of infectious virus and allows the interrogation of many antibodies in a single experiment due to growth of phage to very high titers. The DMS phage display approach is based on binding of displayed peptide sequences with a candidate binding molecule. Thus, in the context of antibodies, the approach does not require, for example, neutralization of a virus by an antibody, allowing application of the method to mapping any antibody/antigen interaction.

(viii) Closing Paragraphs. “Specifically binds” refers to an association of a binding domain (of, for example, a CAR binding domain or a nanoparticle selected cell targeting ligand) to its cognate binding molecule with an affinity or Ka (i.e., an equilibrium association constant of a particular binding interaction with units of 1/M) equal to or greater than 105 M-1, while not significantly associating with any other molecules or components in a relevant environment sample. Binding domains may be classified as “high affinity” or “low affinity”. In particular embodiments, “high affinity” binding domains refer to those binding domains with a Ka of at least 107 M-1, at least 108 M-1, at least 109 M-1, at least 1010 M-1, at least 1011 M-1, at least 1012 M-1, or at least 1013 M-1. In particular embodiments, “low affinity” binding domains refer to those binding domains with a Ka of up to 107 M-1, up to 106 M-1, up to 105 M-1. Alternatively, affinity may be defined as an equilibrium dissociation constant (Kd) of a particular binding interaction with units of M (e.g., 10-5 M to 10-13 M). In certain embodiments, a binding domain may have “enhanced affinity,” which refers to a selected or engineered binding domains with stronger binding to a cognate binding molecule than a wild type (or parent) binding domain. For example, enhanced affinity may be due to a Ka (equilibrium association constant) for the cognate binding molecule that is higher than the reference binding domain or due to a Kd (dissociation constant) for the cognate binding molecule that is less than that of the reference binding domain, or due to an off-rate (Koff) for the cognate binding molecule that is less than that of the reference binding domain. A variety of assays are known for detecting binding domains that specifically bind a particular cognate binding molecule as well as determining binding affinities, such as Western blot, ELISA, and BIACORE® analysis (see also, e.g., Scatchard, et al., 1949, Ann. N.Y. Acad. Sci. 51:660; and U.S. Pat. Nos. 5,283,173, 5,468,614, or the equivalent).

Reference to residues and mutation positions herein refer to HXB2 numbering, unless clearly noted to the contrary.

As will be understood by one of ordinary skill in the art, each embodiment disclosed herein can comprise, consist essentially of or consist of its particular stated element, step, ingredient or component. Thus, the terms “include” or “including” should be interpreted to recite: “comprise, consist of, or consist essentially of.” The transition term “comprise” or “comprises” means includes, but is not limited to, and allows for the inclusion of unspecified elements, steps, ingredients, or components, even in major amounts. The transitional phrase “consisting of” excludes any element, step, ingredient or component not specified. The transition phrase “consisting essentially of” limits the scope of the embodiment to the specified elements, steps, ingredients or components and to those that do not materially affect the embodiment. A material effect would cause an inability to map protein residues responsible for particular protein/protein interactions.

Unless otherwise indicated, all numbers expressing quantities of ingredients, properties such as molecular weight, reaction conditions, and so forth used in the specification and claims are to be understood as being modified in all instances by the term “about.” Accordingly, unless indicated to the contrary, the numerical parameters set forth in the specification and attached claims are approximations that may vary depending upon the desired properties sought to be obtained by the present invention. At the very least, and not as an attempt to limit the application of the doctrine of equivalents to the scope of the claims, each numerical parameter should at least be construed in light of the number of reported significant digits and by applying ordinary rounding techniques. When further clarity is required, the term “about” has the meaning reasonably ascribed to it by a person skilled in the art when used in conjunction with a stated numerical value or range, i.e. denoting somewhat more or somewhat less than the stated value or range, to within a range of ±20% of the stated value; ±19% of the stated value; ±18% of the stated value; ±17% of the stated value; ±16% of the stated value; ±15% of the stated value; ±14% of the stated value; ±13% of the stated value; ±12% of the stated value; ±11% of the stated value; ±10% of the stated value; ±9% of the stated value; ±8% of the stated value; ±7% of the stated value; ±6% of the stated value; ±5% of the stated value; ±4% of the stated value; ±3% of the stated value; ±2% of the stated value; or ±1% of the stated value.

Notwithstanding that the numerical ranges and parameters setting forth the broad scope of the invention are approximations, the numerical values set forth in the specific examples are reported as precisely as possible. Any numerical value, however, inherently contains certain errors necessarily resulting from the standard deviation found in their respective testing measurements.

The terms “a,” “an,” “the” and similar referents used in the context of describing the invention (especially in the context of the following claims) are to be construed to cover both the singular and the plural, unless otherwise indicated herein or clearly contradicted by context. Recitation of ranges of values herein is merely intended to serve as a shorthand method of referring individually to each separate value falling within the range. Unless otherwise indicated herein, each individual value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein is intended merely to better illuminate the invention and does not pose a limitation on the scope of the invention otherwise claimed. No language in the specification should be construed as indicating any non-claimed element essential to the practice of the invention.

Groupings of alternative elements or embodiments of the invention disclosed herein are not to be construed as limitations. Each group member may be referred to and claimed individually or in any combination with other members of the group or other elements found herein. It is anticipated that one or more members of a group may be included in, or deleted from, a group for reasons of convenience and/or patentability. When any such inclusion or deletion occurs, the specification is deemed to contain the group as modified thus fulfilling the written description of all Markush groups used in the appended claims.

Certain embodiments of this invention are described herein, including the best mode known to the inventors for carrying out the invention. Of course, variations on these described embodiments will become apparent to those of ordinary skill in the art upon reading the foregoing description. The inventor expects skilled artisans to employ such variations as appropriate, and the inventors intend for the invention to be practiced otherwise than specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

Furthermore, numerous references have been made to patents, printed publications, journal articles and other written text throughout this specification (referenced materials herein). Each of the referenced materials are individually incorporated herein by reference in their entirety for their referenced teaching.

In closing, it is to be understood that the embodiments of the invention disclosed herein are illustrative of the principles of the present invention. Other modifications that may be employed are within the scope of the invention. Thus, by way of example, but not of limitation, alternative configurations of the present invention may be utilized in accordance with the teachings herein. Accordingly, the present invention is not limited to that precisely as shown and described.

The particulars shown herein are by way of example and for purposes of illustrative discussion of the preferred embodiments of the present invention only and are presented in the cause of providing what is believed to be the most useful and readily understood description of the principles and conceptual aspects of various embodiments of the invention. In this regard, no attempt is made to show structural details of the invention in more detail than is necessary for the fundamental understanding of the invention, the description taken with the drawings and/or examples making apparent to those skilled in the art how the several forms of the invention may be embodied in practice.

Definitions and explanations used in the present disclosure are meant and intended to be controlling in any future construction unless clearly and unambiguously modified in the examples or when application of the meaning renders any construction meaningless or essentially meaningless. In cases where the construction of the term would render it meaningless or essentially meaningless, the definition should be taken from Webster's Dictionary, 3rd Edition or a dictionary known to those of ordinary skill in the art, such as the Oxford Dictionary of Biochemistry and Molecular Biology (Eds. Attwood T et al., Oxford University Press, Oxford, 2006).

What is claimed is:

1. A method of performing protein residue mapping comprising:
obtaining a phage library expressing deep mutational scanning (DMS) proteins or peptides;
incubating the phage library expressing the DMS proteins or peptides in a solution comprising a candidate binding molecule;
separating phage bound to the candidate binding molecule from phage not bound to the candidate binding molecule using immunoprecipitation;
lysing and sequencing nucleotides of the bound and/or unbound phage; and
determining residues responsible for the binding or non-binding of phage to the candidate binding molecule based on the sequencing;
thereby performing protein residue mapping.

2. The method of claim 1, wherein the DMS proteins or peptides are selected from a DMS library.

3. The method of claim 1, wherein the DMS proteins or peptides comprise all peptides in the DMS library.

4. The methods of claim 1, wherein the DMS proteins or peptides are derived from a protein of interest selected from a viral protein, a bacterial protein, a fungal protein, or a cancer cell antigen.

5. The method of claim 4, wherein the viral protein comprises a human immunodeficiency virus-1 (HIV-1) viral protein, an HIV-2 viral protein, a simian immunodeficiency virus (SIV) viral protein, an influenza virus viral protein, an Ebola virus viral protein, a coronavirus (CoV) viral protein, a Lassa virus viral protein, a Nipah virus viral protein, a Chikungunya virus viral protein, a Hendra virus viral protein, a hepatitis B virus viral protein, a hepatitis C virus viral protein, a measles virus viral protein, a Rabies virus viral protein, a respiratory syncytial virus (RSV) viral protein, a Zika virus viral protein, a Dengue virus viral protein, or a Herpes virus viral protein.

6. The method of claim 5, wherein the CoV viral protein comprises a Wuhan CoV (COVID) viral protein, a severe acute respiratory syndrome CoV (SARS-CoV) viral protein or a Middle East respiratory syndrome coronavirus (MERS-CoV) viral protein.

7. The method of claim 4, wherein the protein of interest comprises a viral entry protein.

8. The method of claim 4, wherein the viral protein is a subunit of a viral entry protein.

9. The method of claim 7, wherein the viral entry protein comprises Chikungunya virus E1 Env or E2 Env; the Ebola glycoprotein (EBOV GP), the Hendra virus F glycoprotein or G glycoprotein; the hepatitis B virus large (L), middle (M), or small (S) protein; the hepatitis C virus glycoprotein E1 or glycoprotein E2; the HIV envelope (Env) protein; the influenza virus hemagglutinin (HA) protein, the Lassa virus envelope glycoprotein (GPC); the measles virus hemagglutinin glycoprotein (H) or fusion glycoprotein F0 (F)); the MERS-CoV Spike (S) protein; the Nipah virus fusion glycoprotein F0 (F) or glycoprotein G); the Rabies virus glycoprotein (RABV G); the RSV fusion glycoprotein F0 (F) or glycoprotein G); or the SARS-CoV Spike (S) protein.

10. The method of claim 8, wherein the subunit of the viral entry protein comprises HIV gp41 and/or gp120.

11. The method of claim 4, wherein the protein of interest comprises BF520.W14.C2; BG505.W6M.C2.T332N; BG505 SOSIP Env trimer; BL035.W6M.ENV.C1; SF162; ZM109F.PB4; C2-94UG114; SIV/mac239; resurfaced Env core protein (RSC3); CD4-binding site defective mutant (RSC3 Δ371I); 2J9C-ZM53_V1V2; a 1FD6-Fc-ZM109_V1V2 scaffold peptide; a V3 consensus peptide of ConA1 and ConB; MN gp41 monomer; ectodomain ZA.1197/MB; Q23; QA013.70I.Env.H1; QA013.385M.Env.R3 677; QB850.73P.C14; QB850.632P.B10; Q461.D1; or QC406.F3.

12. The method of claim 4, wherein the protein of interest comprises a bacterial protein derived from anthrax, gram-negative bacilli, chlamydia, diptheria, Helicobacter pylori, Mycobacterium tuberculosis, pertussis toxin, pneumococcus, rickettsiae, staphylococcus, streptococcus or tetanus.

13. The method of claim 4, wherein the protein of interest comprises anthrax protective antigen, lipopolysaccharides, diptheria toxin, mycolic acid, heat shock protein 65 (HSP65), the 30 kDa major secreted protein, antigen 85A, hemagglutinin, pertactin, FIM2, FIM3, adenylate cyclase, pneumolysin, pneumococcal capsular polysaccharides, rompA, M proteins or tetanus toxin.

14. The method of claim 4, wherein the protein of interest comprises a fungal protein derived from candida, coccidiodes, cryptococcus, histoplasma, leishmania, plasmodium, protozoa, parasites, schistosomae, tinea, toxoplasma, or Trypanosoma cruzi.

15. The method of claim 4, wherein the protein of interest comprises spherule antigens, capsular polysaccharides, heat shock protein 60 (HSP60), gp63, lipophosphoglycan, merozoite surface antigens, sporozoite surface antigens, circumsporozoite antigens, gametocyte/gamete surface antigens, the blood-stage antigen pf 155/RESA, glutathione-S-transferase, paramyosin, trichophytin, SAG-1, p30, the Trypanosoma cruzi 75-77 kDa antigen or the Trypanosoma cruzi 56 kDa antigen.

16. The method of claim 4, wherein the protein of interest comprises a cancer antigen protein derived from, for example, brain cancer, breast cancer, colon cancer, H BV-induced hepatocellular carcinoma, intestinal cancer, kidney cancer, leukemia, liver cancer, lung cancer, lymphoma, melanoma, mesothelioma, multiple myeloma, ovarian cancer, pancreatic cancer, prostate cancer, renal cell carcinoma, stem cell cancer, stomach cancer, throat cancer, or uterine cancer.

17. The method of claim 4, wherein the protein of interest comprises A33, β-catenin, BAGE, Bcl-2, BCMA, c-Met, CA19-9, CA125, CAIX, CD5, CD19, CD20, CD21, CD22, CD24, CD33, CD37, CD45, CD123, CD133, CEA, CS-1, cyclin 1, DAGE, EBNA, EGFR, ephrinB2, ERBB2, estrogen receptor, FAP, ferritin, folate-binding protein, GAGE, G250, GD2, GM2, gp75, gp100 (Pmel 17), HER-2/neu, HPV E6, HPV E7, Ki-67, LRP, mesothelin, p53, PRAME, progesterone receptor, PSA, PSCA, PSMA, MAGE, MART, mesothelin, MUC, MUM-1-B, myc, NYESO-1, ras, RORI, SV40 T, survivin, tenascin, TSTA tyrosinase, VEGF, or WT1

18. The method of claim 4, wherein the DMS proteins or peptides within the DMS library for the protein of interest substitute at least 95% of amino acid residues of the protein of interest with at least 17 amino acid substitutions.

19. The method of claim 4, wherein the DMS proteins or peptides within the DMS library for the protein of interest substitute all amino acid residues of the protein of interest with 19 amino acid substitutions.

20. The method of claim 4, wherein the DMS peptides are staggered fragments of the protein of interest.

21. The method of claim 20, wherein the staggered fragments are formed by moving 1-3 amino acid residue position down the length of the protein of interest while maintaining the same length of peptide fragments.

22. The method of claim 20, wherein the staggered fragments are formed by moving 1 amino acid residue position down the length of the protein of interest while maintaining the same length of peptide fragments.

23. The method of claim 1, wherein the DMS peptides are 50 amino acids or fewer in length.

24. The method of claim 20, wherein the staggered fragments are 28-33 amino acid residues in length.

25. The method of claim 1, wherein the DMS proteins or peptides are not barcoded.

26. The method of claim 1, wherein DMS proteins or peptides further comprise a functional sequence.

27. The method of claim 26, wherein the functional sequence is selected from a transport sequence, a buffer sequence, a tag sequence, and/or a selectable marker.

28. The method of claim 27, wherein the functional sequence comprises a transport sequence.

29. The method of claim 28, wherein the transport sequence comprises a minor coat protein, a major coat protein, a gene 10 protein, or a capsid D protein.

30. The method of claim 27, wherein the functional sequence comprises a buffer sequence.

31. The method of claim 30, wherein the buffer sequence comprises a flexible linker.

32. The method of claim 31, wherein the flexible linker comprises a (Gly)n (SEQ ID NO: 75), (Ser)n, (SEQ ID NO: 76), or (Ala)n (SEQ ID NO: 77) flexible linker wherein =4 or more.

33. The method of claim 31, wherein the flexible linker comprises a Gly-Ser linker or a Gly-Ala linker.

34. The method of claim 33, wherein the Gly-Ser linker is selected from the group consisting of (Gly4Ser)3 (SEQ ID NO: 74), (Gly-Ser)n (SEQ ID NO: 78), (Gly-Ser-Ser-Gly)n (SEQ ID NO: 79), (Gly-Ser-Gly)n (SEQ ID NO: 80), (Gly-Ser-Ser)n (SEQ ID NO: 81), or any combination thereof, where n=1, 2, 3, 4, 5, 6, 7, 8, 9, or 10.

35. The method of claim 33, wherein the Gly-Ser linker is (Gly4Ser)3 (SEQ ID NO: 74).

36. The method of claim 1, wherein the candidate binding molecule comprises an antibody, ligand, peptide, peptide aptamer, enzyme substrate, or receptor.

37. The method of claim 36, wherein the candidate binding molecule comprises an antibody.

38. The method of claim 37, wherein the antibody comprises a human, mammalian, camelid, or shark antibody.

39. The method of claim 37, wherein the antibody comprises an antibody that binds gp41.

40. The method of claim 39, wherein the antibody that binds gp41 comprises a monoclonal antibody selected from QA255.006, QA255.016, QA255.167, QA255.072, and QA255.221.

41. The method of claim 37, wherein the antibody comprises an antibody that binds gp120.

42. The method of claim 41, wherein the antibody that binds gp120 is selected from QA255.105 and QA255.157.

43. The method of claim 37, wherein the antibody comprises VRC01, PG9, PGT121, 4E10, 50-69, 240-D, 246-D, 5F3, 2F5, 167-D, F240, D5, leronlimab, PRO 542, ibalizumab, b12, PEHRG214, 3BNC117, 131-2G, 12G5, MAB8582, MAB8581, MCA490, 104E5, 38F10, 14G3, 90D3, 56E11, 69F6, c13C6, c2G4, c4G7, c1H3, LCA60, REGN3051, REGN3048, 37.2D, 8.9F, 19.7E, 37.7H, 12.1F, or m102.4.

44. The method of claim 37, wherein the antibody comprises leronlimab, PRO 542, ibalizumab, clone 131-2G, clone 12G5, MAB8582, MAB8581, MCA490, 104E5, 38F10, 14G3, 90D3, 56E11, 69F6, c13C6, c2G4, c4G7, c1H3, LCA60, REGN3051, REGN3048, 37.2D, 8.9F, 19.7E, 37.7H, 12.1F, m102.4, or mAb Fi6_v3.

45. The method of claim 1, wherein the phage comprise filamentous phage or bacteriophage.

46. The method of claim 1, wherein the phage comprise f1, fd, M13, T7, T4, or lambdoid phage.

47. The method of claim 1, further comprising cloning nucleotides encoding the DMS proteins or peptides into phage to create the phage library.

48. The method of claim 1, further comprising validating the phage library by sequencing to generate baseline reference level of clones within the library.

49. The method of claim 1, wherein the incubating occurs within a single tube or well.

50. The method of claim 1, wherein the separating using immunoprecipitation comprises adding magnetic beads with binding domains that bind a complex of a phage bound to the candidate binding molecule to the solution and utilizing a source of magnetism to isolate the magnetic beads.

51. The method of claim 1, wherein the sequencing comprises next-generation sequencing (NGS).

52. The method of claim 51, wherein the NGS comprises automated Sanger sequencing, sequencing by synthesis, pyrosequencing, sequencing by ligation, rolling amplification sequencing, single molecule sequencing, or nanopore sequencing.

53. The method of claim 1 wherein the determining residues responsible for the binding or non-binding of phage to the candidate binding molecule based on the sequencing comprises determining an enrichment factor of DMS proteins or peptides and a reproducibility threshold.

54. The method of claim 53, further comprising classifying each DMS proteins or peptide that is enriched above the reproducibility threshold as a hit within a bioinformatics analysis.

55. The method of claim 54, further comprising aligning the DMS proteins or peptides classified as hits and identifying regions of overlap between the aligned proteins or peptides.

56. A kit for performing protein residue mapping comprising a phage library expressing deep mutational scanning (DMS) proteins or peptides.

57. The kit of claim 56, wherein the phage comprise filamentous phage or bacteriophage.

58. The kit of claim 56, wherein the phage comprise f1, fd, M13, T7, T4, or lambdoid phage.

59. The kit of claim 56, further comprising magnetic beads associated with a binding domain.

60. The kit of claim 56, further comprising a candidate binding molecule.

61. The kit of claim 60, wherein the candidate binding molecule comprises an antibody, ligand, peptide, peptide aptamer, enzyme substrate, or receptor.

62. The kit of claim 60, wherein the candidate binding molecule comprises an antibody.

63. The kit of claim 62, wherein the antibody comprises an antibody that binds gp120 or gp41.

64. The kit of claim 62, wherein the antibody comprises QA255.006, QA255.016, QA255.167, QA255.072, QA255.221, QA255.105, QA255.157, VRC01, PG9, PGT121, 4E10, 50-69, 240-D, 246-D, 5F3, 2F5, 167-D, F240, D5, leronlimab, PRO 542, ibalizumab, b12, PEHRG214, 3BNC117, 131-2G, 12G5, MAB8582, MAB8581, MCA490, 104E5, 38F10, 14G3, 90D3, 56E11, 69F6, c13C6, c2G4, c4G7, c1H3, LCA60, REGN3051, REGN3048, 37.2D, 8.9F, 19.7E, 37.7H, 12.1F, m102.4, leronlimab, PRO 542, ibalizumab, clone 131-2G, clone 12G5, MAB8582, MAB8581, MCA490, 104E5, 38F10, 14G3, 90D3, 56E11, 69F6, c13C6, c2G4, c4G7, c1H3, LCA60, REGN3051, REGN3048, 37.2D, 8.9F, 19.7E, 37.7H, 12.1F, m102.4, or mAb Fi6_v3.

65. The kit of claim 56, wherein the DMS proteins or peptides are derived from a protein of interest selected from a viral protein, a bacterial protein, a fungal protein, or a cancer cell antigen.

66. The kit of claim 65, wherein the viral protein comprises a human immunodeficiency virus-1 (HIV-1) viral protein, an HIV-2 viral protein, a simian immunodeficiency virus (SIV) viral protein, an influenza virus viral protein, an Ebola virus viral protein, a coronavirus (CoV) viral protein, a Lassa virus viral protein, a Nipah virus viral protein, a Chikungunya virus viral protein, a Hendra virus viral protein, a hepatitis B virus viral protein, a hepatitis C virus viral protein, a measles virus viral protein, a Rabies virus viral protein, a respiratory syncytial virus (RSV) viral protein, a Zika virus viral protein, a Dengue virus viral protein, or a Herpes virus viral protein.

67. The kit of claim 66, wherein the CoV viral protein comprises a Wuhan CoV (COVID) viral protein, a severe acute respiratory syndrome CoV (SARS-CoV) viral protein or a Middle East respiratory syndrome coronavirus (MERS-CoV) viral protein.

68. The kit of claim 65, wherein the protein of interest comprises a viral entry protein.

69. The kit of claim 65, wherein the viral protein is a subunit of a viral entry protein.

70. The kit of claim 68, wherein the viral entry protein comprises Chikungunya virus E1 Env or E2 Env; the Ebola glycoprotein (EBOV GP), the Hendra virus F glycoprotein or G glycoprotein; the hepatitis B virus large (L), middle (M), or small (S) protein; the hepatitis C virus glycoprotein E1 or glycoprotein E2; the HIV envelope (Env) protein; the influenza virus hemagglutinin (HA) protein, the Lassa virus envelope glycoprotein (GPC); the measles virus hemagglutinin glycoprotein (H) or fusion glycoprotein F0 (F)); the MERS-CoV Spike (S) protein; the Nipah virus fusion glycoprotein F0 (F) or glycoprotein G); the Rabies virus glycoprotein (RABV G); the RSV fusion glycoprotein F0 (F) or glycoprotein G); or the SARS-CoV Spike (S) protein.

71. The kit of claim 69, wherein the subunit of the viral entry protein comprises HIV gp41 and/or gp120.

72. The kit of claim 65, wherein the protein of interest comprises BF520.W14.C2; BG505.W6M.C2.T332N; BG505 SOSIP Env trimer; BL035.W6M.ENV.C1; SF162; ZM109F.PB4; C2-94UG114; SIV/mac239; resurfaced Env core protein (RSC3); CD4-binding site defective mutant; 2J9C-ZM53_V1V2; a 1FD6-Fc-ZM109_V1V2 scaffold peptide; a V3 consensus peptide of ConA1 and ConB; MN gp41 monomer; ectodomain ZA.1197/MB; Q23; QA013.70I.Env.H1; QA013.385M.Env.R3 677; QB850.73P.C14; QB850.632P.B10; Q461.D1; or QC406.F3.

73. The kit of claim 65, wherein the protein of interest comprises a bacterial protein derived from anthrax, gram-negative bacilli, chlamydia, diptheria, Helicobacter pylori, Mycobacterium tuberculosis, pertussis toxin, pneumococcus, rickettsiae, staphylococcus, streptococcus or tetanus.

74. The kit of claim 65, wherein the protein of interest comprises anthrax protective antigen, lipopolysaccharides, diptheria toxin, mycolic acid, heat shock protein 65 (HSP65), the 30 kDa major secreted protein, antigen 85A, hemagglutinin, pertactin, FIM2, FIM3, adenylate cyclase, pneumolysin, pneumococcal capsular polysaccharides, rompA, M proteins or tetanus toxin.

75. The kit of claim 65, wherein the protein of interest comprises a fungal protein derived from candida, coccidiodes, cryptococcus, histoplasma, leishmania, plasmodium, protozoa, parasites, schistosomae, tinea, toxoplasma, or Trypanosoma cruzi.

76. The kit of claim 65, wherein the protein of interest comprises spherule antigens, capsular polysaccharides, heat shock protein 60 (HSP60), gp63, lipophosphoglycan, merozoite surface antigens, sporozoite surface antigens, circumsporozoite antigens, gametocyte/gamete surface antigens, the blood-stage antigen pf 155/RESA, glutathione-S-transferase, paramyosin, trichophytin, SAG-1, p30, the Trypanosoma cruzi 75-77 kDa antigen or the Trypanosoma cruzi 56 kDa antigen.

77. The kit of claim 65, wherein the protein of interest comprises a cancer antigen protein derived from, for example, brain cancer, breast cancer, colon cancer, HBV-induced hepatocellular carcinoma, intestinal cancer, kidney cancer, leukemia, liver cancer, lung cancer, lymphoma, melanoma, mesothelioma, multiple myeloma, ovarian cancer, pancreatic cancer, prostate cancer, renal cell carcinoma, stem cell cancer, stomach cancer, throat cancer, or uterine cancer.

78. The kit of claim 65, wherein the protein of interest comprises A33, β-catenin, BAGE, Bcl-2, BCMA, c-Met, CA19-9, CA125, CAIX, CD5, CD19, CD20, CD21, CD22, CD24, CD33, CD37, CD45, CD123, CD133, CEA, CS-1, cyclin B1, DAGE, EBNA, EGFR, ephrinB2, ERBB2, estrogen receptor, FAP, ferritin, folate-binding protein, GAGE, G250, GD2, GM2, gp75, gp100 (Pmel 17), HER-2/neu, HPV E6, HPV E7, Ki-67, LRP, mesothelin, p53, PRAME, progesterone receptor, PSA, PSCA, PSMA, MAGE, MART, mesothelin, MUC, MUM-1-B, myc, NYESO-1, ras, RORI, SV40 T, survivin, tenascin, TSTA tyrosinase, VEGF, or WT1

79. The kit of claim 56, wherein the DMS proteins or peptides within the DMS library for the protein of interest substitute at least 95% of amino acid residues of the protein of interest with at least 17 amino acid substitutions.

80. The kit of claim 56, wherein the DMS proteins or peptides within the DMS library for the protein of interest substitute all amino acid residues of the protein of interest with 19 amino acid substitutions.

81. The kit of claim 56, wherein the DMS peptides are staggered fragments of the protein of interest.

82. The kit of claim 81, wherein the staggered fragments are formed by moving 1-3 amino acid residue position down the length of the protein of interest while maintaining the same length of peptide fragments.

83. The kit of claim 81, wherein the staggered fragments are formed by moving 1 amino acid residue position down the length of the protein of interest while maintaining the same length of peptide fragments.

84. The kit of claim 56, wherein the DMS peptides are 50 amino acids or fewer in length.

85. The kit of claim 81, wherein the staggered fragments are 28-33 amino acid residues in length.

86. The kit of claim 56, wherein the DMS proteins or peptides are not barcoded.

87. The kit of claim 56, wherein DMS proteins or peptides further comprise a functional sequence selected from a transport sequence, a buffer sequence, a tag sequence, and/or a selectable marker.


SEQUENCE LISTING (96 sequences)

SEQ ID NO: 1 - protein, 67 aa, Human immunodeficiency virus
LTVWGIKQLQARILAVERYLKDQQLLGIWGCSGKLICTTAVPWNASWSNKSLEQIWNHTTWMEWDRE

SEQ ID NO: 2 - protein, 39 aa, Artificial Sequence
  Phage display peptides for QA255
QQLLGIWGCSGKLICTTNVPWNSSWSNKNQSEIWDRMTW

SEQ ID NO: 3 - protein, 39 aa, Artificial Sequence
  Phage display peptides for QA255
VERYLRDQQLLGIWGCSGKLICTTNVPWNSSWSNKSQNE

SEQ ID NO: 4 - protein, 39 aa, Artificial Sequence
  Phage display peptides for QA255
ERYLRDQQLLGIWGCSGKLICTTNVPWNSSWSNRNLSEI

SEQ ID NO: 5 - protein, 39 aa, Artificial Sequence
  Phage display peptides for QA255
KDQQLLGIWGCSGKLICTTNVPWNSSWSNKSQNEIWDNM

SEQ ID NO: 6 - protein, 39 aa, Artificial Sequence
  Phage display peptides for QA255
LALERYLQDQQLLGIWGCSGKLICATTVPWNSSWSNKTQ

SEQ ID NO: 7 - protein, 39 aa, Artificial Sequence
  Phage display peptides for QA255
DQQLLGIWGCSGKLICTTAVPWNSSWSNKSQEDIWDNMT

SEQ ID NO: 8 - protein, 39 aa, Artificial Sequence
  Phage display peptides for QA255
QLLGIWGCSGKLICTTNVPWNSSWSNKSLDEIWNNMTWL

SEQ ID NO: 9 - protein, 39 aa, Artificial Sequence
  Phage display peptides for QA255
LLGIWGCSGKLICTTNVPWNSSWSNKSQDEIWGNMTWLQ

SEQ ID NO: 10 - protein, 39 aa, Artificial Sequence
  Phage display peptides for QA255
RVLAVERYLKDQQLLGIWGCSGKLICTTNVPWNSSWSNK

SEQ ID NO: 11 - protein, 39 aa, Artificial Sequence
  Phage display peptides for QA255
VLAIERYLQDQQLLGIWGCSGKLICTTAVPWNSSWSNKS

SEQ ID NO: 12 - protein, 39 aa, Artificial Sequence
  Phage display peptides for QA255
LQARVLAVERYLKDQQLLGIWGCSGKLICTTAVPWNASW

SEQ ID NO: 13 - protein, 39 aa, Artificial Sequence
  Phage display peptides for QA255
ARVLAVERYLKDQQLLGIWGCSGKLICTTSVPWNSSWSN

SEQ ID NO: 14 - protein, 39 aa, Artificial Sequence
  Phage display peptides for QA255
QLQARVLAVESYLKDQQLLGIWGCSGKHICTTTVPWNSS

SEQ ID NO: 15 - protein, 39 aa, Artificial Sequence
  Phage display peptides for QA255
WGIKQLQARVLAVERYLRDQQLLGIWGCSGKLICTTNVP

SEQ ID NO: 16 - protein, 39 aa, Artificial Sequence
  Phage display peptides for QA255
GIKQLQARVLAVERYLKDQQLLGIWGCSGKLICTTNVPW

SEQ ID NO: 17 - protein, 39 aa, Artificial Sequence
  Phage display peptides for QA255
LTVWGIKQLQARVLAVERYLKDQQLLGIWGCSGKLICTT

SEQ ID NO: 18 - protein, 39 aa, Artificial Sequence
  Phage display peptides for QA255
QARILAVERYLKDQQLLGIWGCSGKHICTTTVPWNSSWS

SEQ ID NO: 19 - protein, 39 aa, Artificial Sequence
  Phage display peptides for QA255
VWGIKQLQARILAVERYLKDQQLLGIWGCSGKLICTTNV

SEQ ID NO: 20 - protein, 39 aa, Artificial Sequence
  Phage display peptides for QA255
TVWGIKQLQTRVLAIERYLKDQQLLGIWGCSGKLICTTA

SEQ ID NO: 21 - protein, 39 aa, Artificial Sequence
  Phage display peptides for QA255
WGCSGKLICTTAVPWNASWSNKSLDEIWDNMTWMEWERE

SEQ ID NO: 22 - protein, 39 aa, Artificial Sequence
  Phage display peptides for QA255
IWGCSGKHICTTTVPWNSSWSNKSLEEIWNNMTWMEWER

SEQ ID NO: 23 - protein, 12 aa, Artificial Sequence
  240-D
WGCSGKLICTTA

SEQ ID NO: 24 - protein, 15 aa, Artificial Sequence
  QA255.067
LLGIWGCSGKLICTT

SEQ ID NO: 25 - protein, 14 aa, Artificial Sequence
  QA255.072
WGCSGKLICTTNVP

SEQ ID NO: 26 - DNA, 58 bp, Artificial Sequence
  R2 forward primer
  misc_feature (30)..(30): n = a, c, g, t/u, unknown, or other
  misc_feature (31)..(31): n = a, c, g, t/u, unknown, or other
  misc_feature (32)..(32): n = a, c, g, t/u, unknown, or other
  misc_feature (33)..(33): n = a, c, g, t/u, unknown, or other
  misc_feature (34)..(34): n = a, c, g, t/u, unknown, or other
  misc_feature (35)..(35): n = a, c, g, t/u, unknown, or other
  misc_feature (36)..(36): n = a, c, g, t/u, unknown, or other
  misc_feature (37)..(37): n = a, c, g, t/u, unknown, or other
aatgatacggcgaccaccgagatctacacnnnnnnnntcgtcggcagcgtctccagtc

SEQ ID NO: 27 - protein, 173 aa, Artificial Sequence
  QA255 HIV envelope
AVGIGAVFIGFLGAAGSTMGAASITLTVQARQLLSGIVQQQSNLLRAIEAQQQMLKLTVWGIKQLQARVLAVERYLKDQQLLGIWGCSGKLICTTSVPWNSSWSNKTQSEIWDNMTWLQWDKEISNYTQTIYRLIEESQIQQEKNEQDLLALDQWASLWTWFDISKWLWYIRI

SEQ ID NO: 28 - protein, 173 aa, Artificial Sequence
  QA255 HIV envelope
AVGIGAVFIGFLGAAGSTMGAASITLTVQARQLLSGIVQQQSNLLRAIEAQQQMLKLTVWGIKQLQARVLAVERYLKDQQLLGIWGCSGKLICTTSVPWNSSWSNKTQSEIWDNMTWLQWDKEISNYTQTIYKLIEESQIQQEKNEQDLLALDQWASLWTWFDISKWLWYIKI

SEQ ID NO: 29 - protein, 173 aa, Artificial Sequence
  QA255 HIV envelope
AVGIGAVFIGFLGAAGSTMGAASITLTVQARQLLSGIVQQQSNLLRAIEAQQHMLKLTVWGIKQLQARVLAVERYLKDQQLLGIWGCSGKLICTTSVPWNSSWSNKTQSEIWDNMTWLQWDKEISNYTQTIYKLIEESQIQQEKNEQDLLALDQWASLWTWFDISKWLWYIRI

SEQ ID NO: 30 - protein, 173 aa, Artificial Sequence
  QA255 HIV envelope
AVGLGAVFFGFLGVAGSTMGAASITLTVQARQLLSGIVQQQSNLLRAIEAQQQMLKLTVWGIKQLQARVLAVERYLKDQQLLGIWGCSGKLICTTSVPWNSSWSNKTQSEIWDNMTWLQWDKEISNYTQIIYELIEESQIQQEKNEQDLLALDQWASLWTWFDISKWLWYIRI

SEQ ID NO: 31 - protein, 173 aa, Artificial Sequence
  QA255 HIV envelope
AVGIGAVFIGFLGAAGSTMGAASITLTAQARQLLSGIVQQQSNLLRAIEAQQQMLKLTVWGIKQLQARVLAVERYLKDQQLLGIWGCSGKLICTTSVPWNSSWSNKTQSEIWDNMTWLQWDKEISNYTQTIYKLIEESQIQQEKNEQDLLALDQWASLWTWFDISKWLWYIKI

SEQ ID NO: 32 - protein, 173 aa, Artificial Sequence
  QA255 HIV envelope
AVGLGAVFFGFLGAAGSTMGAASITLTVQARQLLSGIVQQQSNLLRAIEAQQQMLKLTVWGIKQLQARVLAVERYLKDQQLLGIWGCSGKLICTTSVPWNSSWSNKTESEIWDNMTWLQWDKEISNYTQTIYELLEKSQIQQEKNEQDLLALDQWASLWTWFDISKWLWYIKI

SEQ ID NO: 33 - protein, 173 aa, Artificial Sequence
  QA255 HIV envelope
AVGLGAVFFGFLGAAGSTMGAASITLTVQARQLLSGIVQQQSNLLRAIEAQQQMLKLTVWGIKQLQARVLAVERYLKDQQLLGIWGCSGKLICTTSVPWNSSWSNKTQSEIWDNMTWLQWDKEISNYTQTIYNLLEESQIQQEKNEQDLLALDQWASLWTWFDISKWLWYIKI

SEQ ID NO: 34 - protein, 173 aa, Artificial Sequence
  QA255 HIV envelope
AVGLGAVFFGFLGAAGSTMGAASITLTVQARQLLSGIVQQQSNLLRAIEAQQQMLKLTVWGIKQLQARVLAVERYLKDQQLLGIWGCSGKLICTTSVPWNSSWSNKTQSEIWDNMTWLQWDKEISNYTQTIYELLEESQIQQEKNEQDLLALDQWASLWTWFDISKWLWYIKI

SEQ ID NO: 35 - protein, 173 aa, Artificial Sequence
  QA255 HIV envelope
AVGLGAVFFGFLGAAGSTMGAASVTLTVQARQLLSGIVQQQSNLLRAIEAQQQMLKLTVWGIKQLQARVLAVERYLKDQQLLGIWGCSGKLICTTSVPWNSSWSNKTQSEIWDNMTWLQWDKEISNYTQTIYELLEESQIQQEKNEQDLLALDQWASLWTWFDISKWLWYIKI

SEQ ID NO: 36 - protein, 174 aa, Artificial Sequence
  QA255 HIV envelope
AVVGLGAVFFGSLGAAGSTMGAASITLAVQARRLLSGIVQQQSNLLRAIEAQQQMLKLTVWGIKQLQARVLAVERYLKDQQLLGIWGCSGKLICTTSVPWNSSWSNKTQSEIWDNMTWLQWDKEISNYTQTIYNLLEESQIQQEKNEQDLLALDQWASLWTWFDISKWLWYIKI

SEQ ID NO: 37 - protein, 173 aa, Artificial Sequence
  QA255 HIV envelope
AVGLGAVFFGFLGAAGSTMGAASITLTVQARQLLSGIVQQQSNLLRAIEAQQQMLKLTVWGIKQLQARVLAVERYLKDQQLLGIWGCSGKLICTTSVPWNSSWSNKTQSEIWDNMTWLQWDKEISNYTQTIYELLEKSQIQQEKNEQDLLALDQWASLWTWFDISKWLWYIRI

SEQ ID NO: 38 - protein, 173 aa, Artificial Sequence
  QA255 HIV envelope
AVGIGAMFIGFLGAAGSTMGAASITLTVQARQLLSGIVQQQSNLLRAIEAQQQMLKLTVWGIKQLQARVLAVERYLKDQQLLGIWGCSGKLICTTSVPWNSSWSNKTQSEIWDNMTWLQWDKEISNYTQTIYKLIEESQIQQEKNEQDLLALDQWASLWTWFDISKWLWYIKI

SEQ ID NO: 39 - protein, 173 aa, Artificial Sequence
  QA255 HIV envelope
AVGLGALFFGFLGAAGSTMGAASITLTVQARQLLSGIVQQQSNLLRAIEAQQQMLKLTVWGIKQLQARVLAVERYLKDQQLLGIWGCSGKLICTTSVPWNSSWSNKTQSEIWDNMTWLQWDKEISNYTQTIYNLLEESQIQQEKNEKDLLALDQWASLWTWFDISKWLWYIKT

SEQ ID NO: 40 - protein, 173 aa, Artificial Sequence
  QA255 HIV envelope
AIGLGALFFGFLGAAGSTMGAASITLTVQARQLLSGIVQQQSNLLRAIEAQQQMLKLTVWGIKRLQARVLAVERYLKDQQLLGIWGCSGKLICTTSVPWNSSWSNKTQKEIWDNMTWLQWDKEISNYTQTIYNLLEESQIQQEKNEKDLLALDQWASLWTWFDISNWLWYIKI

SEQ ID NO: 41 - protein, 173 aa, Artificial Sequence
  QA255 HIV envelope
AIGLGALFFGFLGAAGSTMGAASITLTVQARQLLSGIVQQQSNLLRAIEAQQQMLKLTVWGIKQLQARVLAVERYLKDQQLLGIWGCSGKLICTTSVPWNSSWSNKTQSEIWDNMTWLQWDKEISNYTQTIYNLLEESQIQQEKNEKDLLALDQWASLWTWFDISNWLWYIKI

SEQ ID NO: 42 - protein, 173 aa, Artificial Sequence
  QA255 HIV envelope
AIGLGALFFGFLGAAGSTMGAASITLTVQARQLLSGIVQQQSNLLRAIEAQQQMLKLTVWGIKQLQARVLAVERYLKDQQLLGIWGCSGKLICTTSVPWNSSWSNKTQREIWDNMTWLQWDKEISNYTQTIYNLLEESQIQQEKNEKDLLALDQWASLWTWFNISNWLWYIKI

SEQ ID NO: 43 - protein, 173 aa, Artificial Sequence
  QA255 HIV envelope
AIGLGALFFGFLGAAGSTMGAASITLTVQARQLLSGIVQQQSNLLRAIEAQQQMLKLTVWGIKQLQARVLAVERYLKDQQLLGIWGCSGKLICTTSVPWNSSWSNKTQGEIWDNMTWLQWDKEISNYTQIIYELLEESQTQQEKNEKDLLALDQWASLWTWFDISNWLWYIKI

SEQ ID NO: 44 - protein, 178 aa, Artificial Sequence
  QA255 HIV envelope
AIGLGALFFGFLGVGFLGAAGSTMGAASITLTVQARQLLSGIVQQQSNLLRAIEAQQQMLKLTVWGIKQLQARVLAVERYLKDQQLLGIWGCSGKLICTTSVPWNSSWSNKTQKEIWDNMTWLQWDKEISNYTQTIYNLLEESQIQQEKNEKDLLALDQWASLWTWFNISNWLWYIKI

SEQ ID NO: 45 - protein, 9 aa, Artificial Sequence
  known epitope for 240D
LLGIWGCSG

SEQ ID NO: 46 - DNA, 66 bp, Artificial Sequence
  R2 reverse primer
  misc_feature (25)..(25): n = a, c, g, t/u, unknown, or other
  misc_feature (26)..(26): n = a, c, g, t/u, unknown, or other
  misc_feature (27)..(27): n = a, c, g, t/u, unknown, or other
  misc_feature (28)..(28): n = a, c, g, t/u, unknown, or other
  misc_feature (29)..(29): n = a, c, g, t/u, unknown, or other
  misc_feature (30)..(30): n = a, c, g, t/u, unknown, or other
  misc_feature (31)..(31): n = a, c, g, t/u, unknown, or other
  misc_feature (32)..(32): n = a, c, g, t/u, unknown, or other
caagcagaagacggcatacgagatnnnnnnnngtctcgtgggctcggagatgtgtataagagacag

SEQ ID NO: 47 - protein, 9 aa, Artificial Sequence
  peptide with tolerated mutation
LLGIWTCSG

SEQ ID NO: 48 - protein, 9 aa, Artificial Sequence
  peptide with tolerated mutation
LIGIWGCSG

SEQ ID NO: 49 - protein, 9 aa, Artificial Sequence
  peptide with tolerated mutation
LLGIWGCTG

SEQ ID NO: 50 - protein, 9 aa, Artificial Sequence
  peptide with mutation that disrupts epitope
LLSIWGCSG

SEQ ID NO: 51 - protein, 9 aa, Artificial Sequence
  peptide with mutation that disrupts epitope
LLGIWGTSG

SEQ ID NO: 52 - protein, 9 aa, Artificial Sequence
  peptide with mutation that disrupts epitope
LPGIWGCSG

SEQ ID NO: 53 - protein, 9 aa, Artificial Sequence
  peptide with mutation that disrupts epitope
LLGIAGCSG

SEQ ID NO: 54 - protein, 13 aa, Artificial Sequence
  Gp41 peptides strain B41
RKSIHIGPGRAFY

SEQ ID NO: 55 - protein, 13 aa, Artificial Sequence
  V3 peptides strain B41
EKSIHIGPGRAFY

SEQ ID NO: 56 - protein, 13 aa, Artificial Sequence
  V3 peptides strain B41
RTSIHIGPGRAFY

SEQ ID NO: 57 - protein, 13 aa, Artificial Sequence
  V3 peptides strain B41
RKSIHIGPGWAFY

SEQ ID NO: 58 - protein, 13 aa, Artificial Sequence
  V3 peptides strain B41
RKSIHIGPGRATY

SEQ ID NO: 59 - protein, 13 aa, Artificial Sequence
  V3 peptides strain B41
RKSIHIGPGRAFK

SEQ ID NO: 60 - protein, 13 aa, Artificial Sequence
  V3 peptides strain B41
RKSIHWGPGRAFY

SEQ ID NO: 61 - protein, 18 aa, Artificial Sequence
  V3 peptides strain B41
LLGIWGCSGKLICTTNVP

SEQ ID NO: 62 - protein, 18 aa, Artificial Sequence
  V3 peptides strain B41
LLGIWGDSGKLICTTNVP

SEQ ID NO: 63 - protein, 18 aa, Artificial Sequence
  V3 peptides strain B41
LKGIWGCSGKLICTTNVP

SEQ ID NO: 64 - protein, 18 aa, Artificial Sequence
  V3 peptides strain B41
LLGIWGCKGKLICTTNVP

SEQ ID NO: 65 - protein, 18 aa, Artificial Sequence
  V3 peptides strain B41
LLGIWGCSGKEICTTNVP

SEQ ID NO: 66 - protein, 6 aa, Artificial Sequence
  His tag
HHHHHH

SEQ ID NO: 67 - protein, 8 aa, Artificial Sequence
  Flaag tag
DYKDDDDK

SEQ ID NO: 68 - protein, 8 aa, Artificial Sequence
  Xpress tag
DLYDDDDK

SEQ ID NO: 69 - protein, 15 aa, Artificial Sequence
  Avi tag
GLNDIFEAQKIEWHE

SEQ ID NO: 70 - protein, 26 aa, Artificial Sequence
  Calmodulin tag
KRRWKKNFIAVSAANRFKKISSSGAL

SEQ ID NO: 71 - protein, 9 aa, Artificial Sequence
  HA tag
YPYDVPDYA

SEQ ID NO: 72 - protein, 10 aa, Artificial Sequence
  Myc tag
EQKLISEEDL

SEQ ID NO: 73 - protein, 8 aa, Artificial Sequence
  Strep tag
WRHPQFGG

SEQ ID NO: 74 - protein, 15 aa, Artificial Sequence
  Gly-Ser linker
GGGGSGGGGSGGGGS

SEQ ID NO: 75 - protein, 10 aa, Artificial Sequence
  Buffer Sequence
  MISC_FEATURE (2)..(10): The amino acids can either be present or absent
GGGGGGGGGG

SEQ ID NO: 76 - protein, 10 aa, Artificial Sequence
  Buffer Sequence
  MISC_FEATURE (2)..(10): The amino acids can either be present or absent
SSSSSSSSSS

SEQ ID NO: 77 - protein, 10 aa, Artificial Sequence
  Buffer Sequence
  MISC_FEATURE (2)..(10): The amino acids can either be present or absent
AAAAAAAAAA

SEQ ID NO: 78 - protein, 20 aa, Artificial Sequence
  Buffer Sequence
  MISC_FEATURE (3)..(20): (GlySer) can either be present or absent
GSGSGSGSGSGSGSGSGSGS

SEQ ID NO: 79 - protein, 40 aa, Artificial Sequence
  Buffer Sequence
  MISC_FEATURE (5)..(40): (GlySerSerGly) can either be present or absent
GSSGGSSGGSSGGSSGGSSGGSSGGSSGGSSGGSSGGSSG

SEQ ID NO: 80 - protein, 30 aa, Artificial Sequence
  Buffer Sequence
  MISC_FEATURE (4)..(30): (GlySerGly) can either be present or absent
GSGGSGGSGGSGGSGGSGGSGGSGGSGGSG

SEQ ID NO: 81 - protein, 30 aa, Artificial Sequence
  Buffer Sequence
  MISC_FEATURE (4)..(30): (GlySerSer) can either be present or absent
GSSGSSGSSGSSGSSGSSGSSGSSGSSGSS

SEQ ID NO: 82 - protein, 20 aa, Artificial Sequence
  Buffer Sequence
  MISC_FEATURE (3)..(20): (GlyAla) can either be present or absent
GAGAGAGAGAGAGAGAGAGA

SEQ ID NO: 83 - protein, 8 aa, Artificial Sequence
  STREP tag II
WSHPQFEK

SEQ ID NO: 84 - protein, 13 aa, Artificial Sequence
  Softag 1
SLAELLNAGLGGS

SEQ ID NO: 85 - protein, 8 aa, Artificial Sequence
  Softag 3
TQDPSRVG

SEQ ID NO: 86 - protein, 14 aa, Artificial Sequence
  V5 tag
GKPIPNPLLGLDST

SEQ ID NO: 87 - DNA, 58 bp, Artificial Sequence
  adapter sequence
aatgatacggcgaccaccgagatctacactctttccctacacgacgctcttccgatct

SEQ ID NO: 88 - DNA, 63 bp, Artificial Sequence
  adapter sequence
  misc_feature (34)..(39): n is a, c, g, or t
gatcggaagagcacacgtctgaactccagtcacnnnnnnatctcgtatgccgtcttctgcttg

SEQ ID NO: 89 - protein, 35 aa, Artificial Sequence
  V3 consensus peptide
CTRPNNNTRKSIRIGPGQAFYATGDIIGDIRQAHC

SEQ ID NO: 90 - protein, 35 aa, Artificial Sequence
  V3 consensus peptide
CTRPNNNTRKSIHIGPGRAFYTTGEIIGDIRQAHC

SEQ ID NO: 91 - DNA, 18 bp, Artificial Sequence
  adapter sequence
aggaattctacgctgagt

SEQ ID NO: 92 - DNA, 16 bp, Artificial Sequence
  adapter sequence
tgatagcaagcttgcc

SEQ ID NO: 93 - DNA, 29 bp, Artificial Sequence
  T7 forward primer
aatgatacggcaggaattctacgctgagt

SEQ ID NO: 94 - DNA, 27 bp, Artificial Sequence
  T7 reverse primer
cgatcagcagaggcaagcttgctatca

SEQ ID NO: 95 - DNA, 34 bp, Artificial Sequence
  R1 forward primer
tcgtcggcagcgtctccagtcaggtgtgatgctc

SEQ ID NO: 96 - DNA, 49 bp, Artificial Sequence
  R1 reverse primer
gtgggctcggagatgtgtataagagacagcaagacccgtttagaggccc